(19) United States
(12) Patent Application Publication
(10) Pub. No.: US 2025/0114369 A1
(43) Pub. Date: Apr. 10, 2025

(54) COMPOUNDS AND METHODS FOR TREATMENT OF VIRAL INFECTIONS

(71) Applicant: Gilead Sciences, Inc., Foster City, CA (US)

(72) Inventors: Mark J. Bartlett, Castro Valley, CA (US); Richard L. Mackman, Millbrae, CA (US); Xianhuang Zeng, Redwood City, CA (US)

(21) Appl. No.: 18/884,419

(22) Filed: Sep. 13, 2024

Related U.S. Application Data
(60) Provisional application No. 63/538,634, filed on Sep. 15, 2023.

(51) Int. Cl. A61K 31/53; A61P 31/12; C07D 407/14

(52) U.S. Cl. CPC A61K 31/53; A61P 31/12; C07D 407/14

(57) ABSTRACT

Compounds of Formula I, Formula A, or Formula B and methods of using said compounds, singly or in combination with additional agents, and salts or pharmaceutical compositions of said compounds for the treatment of viral infections are disclosed.

### CROSS REFERENCE TO RELATED APPLICATIONS

This application claims the benefit of U.S. Provisional Application No. 63/538,634, filed Sep. 15, 2023, which is incorporated by reference herein in its entirety.

### BACKGROUND

There is a need for compounds and methods for treating viral infections, for example paramyxoviridae, pneumoviridae, picornaviridae, flaviviridae, filoviridae, arenaviridae, orthomyxovirus, and coronaviridae infections. The present disclosure addresses these and other needs.

The oral route is a preferred route for daily drug administration, due to its advantages, such as non-invasiveness, patient compliance, and convenience of drug administration. Nevertheless, oral administration can be limited due to poor physicochemical properties of the drug molecule, including low aqueous solubility between pH2 and pH7, instability, low permeability, and rapid metabolism, all of which can combine to result in low and irregular oral bioavailability. Oral bioavailability (F %) is the fraction of an oral administered drug that reaches systemic circulation relative to the same dose delivery by intravenous administration. After intravenous administration, a drug is directly and fully available in the bloodstream and can be distributed via systemic circulation to the point where a pharmacological effect takes place. If a drug is administered orally, it has to survive the intestinal fluid, cross further barriers such as the gastero-intestinal (GI) cell layer and then the liver in order to reach the systemic circulation, which can significantly reduce the amount of administered drug that reaches the bloodstream. Oral bioavailability is therefore an important property in drug design and development. A high oral bioavailability reduces the required amount of an administered drug that would be necessary to achieve a desired pharmacological effect and therefore could reduce the risk of side-effects and toxicity during the absorption process. The present disclosure also provides compounds with combined solubility, stability and permeability properties leading to improved oral bioavailability.

### SUMMARY

The instant disclosure provides compounds of Formula I:

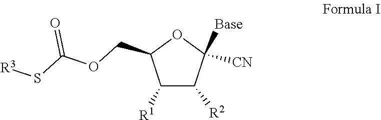

or a pharmaceutically acceptable salt thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O— or —OCR⁶O—;
R³, R⁴, and R⁵ are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R³, R⁴, and R⁵ are each, independently, optionally substituted with one, two or three substituents independently selected from the group consisting of halogen, cyano, —N₃, —OR⁷, —NR⁸R⁹, and phenyl optionally substituted with one, two or three substituents independently selected from halo, cyano, and C₁-C₆ alkyl;
R⁶ is H, C₁-C₆ alkyl or C₆-C₁₀ aryl;
each R⁷ is independently H, C₁-C₆ alkyl, C₁-C₆ haloalkyl, and C₃-C₆ cycloalkyl;
each R⁸ is independently H, C₁-C₆ alkyl, C₁-C₆ haloalkyl, and C₃-C₆ cycloalkyl;
each R⁹ is independently H, C₁-C₆ alkyl, C₁-C₆ haloalkyl, and C₃-C₆ cycloalkyl;
Base is

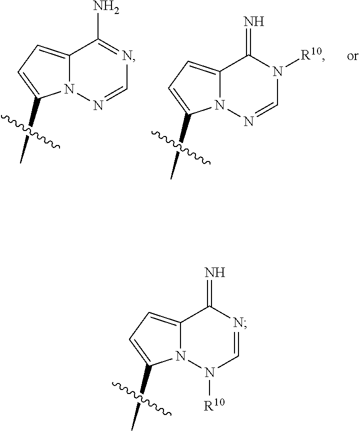

and
R¹⁰ is C₁-C₆ alkyl substituted with —OP(O)(OH)₂.

The instant disclosure also provides compounds of Formula A:

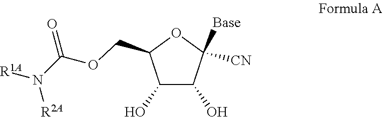

or a pharmaceutically acceptable salt thereof, wherein:
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
   wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —C(═O)R^{3A}, —C(═O)OR^{3A}, —NR^{4A}R^{5A}, C₁-C₆ alkyl, and phenyl;
      wherein the substituent C₁-C₆ alkyl of R^{1A} and R^{2A} is optionally substituted with one, two or three substituents independently selected from halo, cyano, and phenyl; and
      the substituent phenyl of R^{1A} and R^{2A} is optionally substituted with one, two or three substituents independently selected from halo, cyano, and C₁-C₆alkyl; or
R^{1A} is H;
R^{2A} is C₁-C₈ alkyl substituted with —C(═O)OR^{3A};
   wherein C₁-C₈ alkyl substituted with —C(═O)OR^{3A} of R^{2A} is optionally further substituted with one or two substituents independently selected from halo, cyano, and phenyl, and —OR^{3A};
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S;
each R^{4A} is independently H, C₁-C₆ alkyl, C₁-C₆ haloalkyl, and C₃-C₆ cycloalkyl;
each R^{5A} is independently H, C₁-C₆ alkyl, C₁-C₆ haloalkyl, and C₃-C₆ cycloalkyl;
Base is

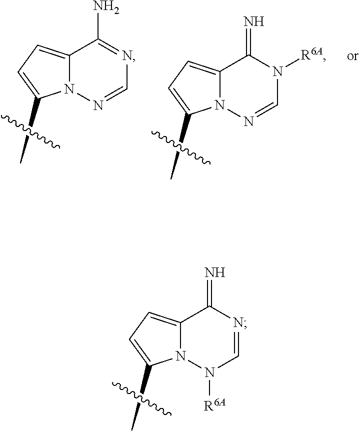

and
R^{6A} is C₁-C₆ alkyl substituted with —OP(O)(OH)₂.

The instant disclosure also provides compounds of Formula B:

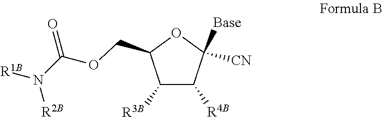

or a pharmaceutically acceptable salt thereof, wherein:
R^{3B} is —OC(═O)R^{5B} or —OC(═O)OR^{5B};
R^{4B} is —OC(═O)R^{6B} or —OC(═O)OR^{6B}; or
R^{3B} and R^{4B} are taken together to form —OC(═O)O— or —OCHR^{7B}O—;
R^{5B} and R^{6B} are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{5B} and R^{6B} are each, independently, optionally substituted with one, two or three substituents independently selected from the group consisting of halogen, cyano, —N₃, —OR^{8B}, —NR^{9B}R^{10B} and phenyl optionally substituted with one, two or three substituents independently selected from halo, cyano, and C₁-C₆ alkyl;
each R^{8B} is independently H, C₁-C₆ alkyl, C₁-C₆ haloalkyl, and C₃-C₆ cycloalkyl;
each R^{9B} is independently H, C₁-C₆ alkyl, C₁-C₆ haloalkyl, and C₃-C₆ cycloalkyl;
each R^{10B} is independently H, C₁-C₆ alkyl, C₁-C₆ haloalkyl, and C₃-C₆ cycloalkyl;
R^{7B} is H, C₁-C₆ alkyl or C₆-C₁₀ aryl;
R^{1B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{1B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl;
R^{2B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{2B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of halogen, cyano, oxo, —N₃, —OR^{11B}, —C(═O)R^{11B}, —C(═O)OR^{11B}, —NR^{12B}R^{13B}, C₁-C₆ alkyl, and phenyl;
      wherein the substituent C₁-C₆ alkyl of RB and R^{2B} is optionally substituted with one, two or three substituents independently selected from halo, cyano, and phenyl; and
      the substituent phenyl of R^{1B} and R^{2B} is optionally substituted with one, two or three substituents independently selected from halo, cyano, and C₁-C₆ alkyl;
each R^{11B} is independently H, C₁-C₆ alkyl, C₁-C₆ haloalkyl, C₃-C₆ cycloalkyl and 3 to 8 membered heterocyclyl containing 1, 2, or 3 heteroatoms selected independently from N, O, and S;
each R^{12B} is independently H, C₁-C₆ alkyl, C₁-C₆ haloalkyl, and C₃-C₆ cycloalkyl;
each R^{13B} is independently H, C₁-C₆ alkyl, C₁-C₆ haloalkyl, and C₃-C₆ cycloalkyl;
Base is

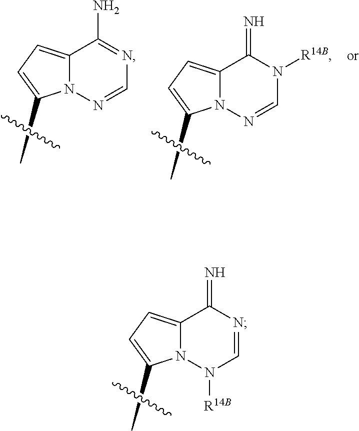

and
R^{14B} is C₁-C₆ alkyl substituted with —OP(O)(OH)₂.

Also provided herein is a pharmaceutical composition comprising a compound disclosed herein, or a pharmaceutically acceptable salt thereof, and one or more pharmaceutically acceptable excipients.

Also provided herein is a method of treating or preventing a viral infection in a human in need thereof, wherein the method comprises administering to the human a compound disclosed herein, or a pharmaceutically acceptable salt thereof, or a pharmaceutical composition disclosed herein.

Also provided herein is a method for manufacturing a medicament for treating or preventing a viral infection in a human in need thereof, characterized in that a compound disclosed herein, or a pharmaceutically acceptable salt thereof.

Also provided herein is a use of a compound disclosed herein, or a pharmaceutically acceptable salt thereof, for the manufacture of a medicament for the treatment or prevention of a viral infection in a human in need thereof.

Also provided herein is a composition comprising a compound disclosed herein, or a pharmaceutically acceptable salt thereof, for use in treatment or prevention of a viral infection in a human in need thereof.

### DETAILED DESCRIPTION OF THE INVENTION

### I. General

The invention relates generally to methods and compounds for treating or preventing viral infections, for example paramyxoviridae, pneumoviridae, picornaviridae, flaviviridae, filoviridae, arenaviridae, orthomyxovirus, and coronaviridae infections.

### II. Definitions

Unless stated otherwise, the following terms and phrases as used herein are intended to have the following meanings:

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₄ alkynyl). The term “alkynyl” also includes those groups having one triple bond and one double bond.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). Examples of suitable haloalkyl groups include, but are not limited to, —CF₃, —CHF₂, —CFH₂, —CH₂CF₃, and the like.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Typical aryl groups include, but are not limited to, radicals derived from benzene (e.g., phenyl), substituted benzene, naphthalene, anthracene, biphenyl, and the like.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring atoms (i.e., 1 to 20 membered heteroaryl), 3 to 12 ring atoms (i.e., 3 to 12 membered heteroaryl) or 3 to 8 carbon ring atoms (3 to 8 membered heteroaryl) or 5 to 6 ring atoms (5 to 6 membered heteroaryl). Examples of heteroaryl groups include pyrimidinyl, purinyl, pyridyl, pyridazinyl, benzothiazolyl, and pyrazolyl. Heteroaryl does not encompass or overlap with aryl as defined above.

“Carbocyclyl” or “carbocyclic ring” refers to a non-aromatic hydrocarbon ring consisting of carbon and hydrogen atoms, having from three to twenty carbon atoms, in certain embodiments having from three to fifteen carbon atoms, in certain embodiments having from three to ten carbon atoms, from three to eight carbon atoms, from three to seven carbon atoms, or from 3 to 6 carbon atoms and which is saturated or partially unsaturated and attached to the rest of the molecule by a single bond. Carbocyclic rings include, for example, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. Carbocyclic rings include cycloalkyl groups.

“Cycloalkyl” refers to a saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, cycloheptyl, and cyclooctyl.

“Heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; “The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. For example, “heterocycle” includes a “carbocycle” as defined herein, wherein one or more (e.g. 1, 2, 3, or 4) carbon atoms have been replaced with a heteroatom (e.g. O, N, or S). As used herein, heterocycle or heterocyclyl has from 3 to 20 ring atoms, 3 to 12 ring atoms, 3 to 10 ring atoms, 3 to 8 ring atoms, or 3 to 6 ring atoms. Ring-forming carbon atoms and heteroatoms of a heterocyle group can be optionally oxidized to form an oxo or sulfido group or other oxidized linkage (e.g., C(O), S(O), C(S), or S(O)₂, N-oxide etc.) or a nitrogen atom can be quaternized. The terms “heterocycle” or “heterocyclyl” includes saturated rings and partially unsaturated rings. Substituted heterocyclyls include, for example, heterocyclic rings substituted with any of the substituents disclosed herein including carbonyl groups. A non-limiting example of a carbonyl substituted heterocyclyl is:

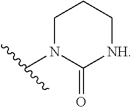 [SMILES: *N1CCCNC1=O]

Example heterocycles include, but are not limited to, tetrahydrofuranyl azetidinyl, and 2-oxo-1,3-dioxol-4-yl.

The term “optionally substituted” in reference to a particular moiety of the compound described herein such as the compound of Formula I, compound of Formula A, or compound of Formula B (e.g., an optionally substituted aryl group) refers to a moiety wherein all substituents are hydrogen or wherein one or more of the hydrogens of the moiety may be replaced by the listed substituents.

Unless otherwise specified, the carbon atoms of the compounds of Formula I, compounds of Formula A, or compounds of Formula B are intended to have a valence of four. If in some chemical structure representations, carbon atoms do not have a sufficient number of variables attached to produce a valence of four, the remaining carbon substituents needed to provide a valence of four should be assumed to be hydrogen.

The term “treating”, as used herein, unless otherwise indicated, means reversing, alleviating, or inhibiting the progress of the disorder or condition to which such term applies, or one or more symptoms of such disorder or condition. The term “treatment”, as used herein, refers to the act of treating, as “treating” is defined immediately above.

“Prevention” or “preventing” means any treatment of a disease or condition that causes the clinical symptoms of the disease or condition not to develop. The compounds and compositions disclosed herein may, in some embodiments, be administered to a subject (including a human) who is at risk of having the disease or condition. As used herein, the terms “preventing” and “prevention” encompass the administration of a compound, composition, or pharmaceutically acceptable salt according to the embodiments disclosed herein pre- or post-exposure of the individual to a virus, but before the appearance of symptoms of the viral infection, and/or prior to the detection of the virus in the blood. The terms also refer to prevention of the appearance of symptoms of the disease and/or to prevent the virus from reaching detectible levels in the blood. The terms include both pre-exposure prophylaxis (PrEP), as well as post-exposure prophylaxis (PEP) and event driven or “on demand” prophylaxis. The terms also refer to prevention of perinatal transmission of a virus from mother to baby, by administration to the mother before giving birth and to the child within the first days of life. The terms also refer to prevention of transmission of a virus through blood transfusion.

The term “therapeutically effective amount”, as used herein, is the amount of a compound described herein (e.g., a compound of Formula I, compound of Formula A, or compound of Formula B) present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the particular compound described herein (e.g., the compound of Formula I, compound of Formula A, or compound of Formula B), the specific activity of the composition, the delivery device employed, the physical characteristics of the composition, its intended use, as well as patient considerations such as severity of the disease state, patient cooperation, etc., and can readily be determined by one skilled in the art based upon the information provided herein.

Any reference to the compounds of the invention described herein also includes a reference to a pharmaceutically acceptable salt thereof. Examples of pharmaceutically acceptable salts of the compounds of the invention include salts derived from an appropriate base, such as an alkali metal or an alkaline earth (for example, Na⁺, Li⁺, K⁺, Ca⁺² and Mg⁺²), ammonium and NR₄ (wherein R is defined herein). Pharmaceutically acceptable salts of a nitrogen atom or an amino group include (a) acid addition salts formed with inorganic acids, for example, hydrochloric acid, hydrobromic acid, sulfuric acid, sulfamic acids, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid, ethanesulfonic acid, lysine, arginine, glutamic acid, glycine, serine, threonine, alanine, isoleucine, leucine and the like; and (c) salts formed from elemental anions for example, chlorine, bromine, and iodine. Pharmaceutically acceptable salts of a compound of a hydroxy group include the anion of said compound in combination with a suitable cation such as Na⁺ and NR₄⁺.

In some embodiments, R is H, (C₁-C₈) alkyl, (C₂-C₈)alkenyl, (C₂-C₈) alkynyl, C₆-C₂₀ aryl, or C₂-C₂₀ heterocyclyl.

For therapeutic use, salts of active ingredients of the compounds of the invention will be pharmaceutically acceptable, i.e., they will be salts derived from a pharmaceutically acceptable acid or base. However, salts of acids or bases which are not pharmaceutically acceptable may also find use, for example, in the preparation or purification of a pharmaceutically acceptable compound. All salts, whether or not derived form a pharmaceutically acceptable acid or base, are within the scope of the present invention.

It is also to be understood that the compositions herein comprise compounds of the invention in their un-ionized, as well as zwitterionic form, and combinations with stoichiometric amounts of water as in hydrates.

It is to be noted that all enantiomers, diastereomers, racemic mixtures, tautomers, polymorphs, and pseudopolymorphs of compounds described herein (e.g., compounds within the scope of Formula I) and pharmaceutically acceptable salts thereof are embraced by the present invention. All mixtures of such enantiomers and diastereomers are within the scope of the present invention.

The compounds of the invention, exemplified by Formula I may have chiral centers, e.g., chiral carbon or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through appropriate techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1, D and L, or (+) and (−) are employed to designate the sign of rotation of plane-polarized light by the compound, with S, (−), or 1 meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The compounds of the invention may also exist as tautomeric isomers in certain cases. Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

Any formula or structure given herein, including Formula I compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds (e.g., compounds of Formula I, compounds of Formula A, or compounds of Formula B) in which from 1 to x hydrogens attached to a carbon atom is/are replaced by deuterium, in which x is the number of hydrogens in the molecule. Such compounds exhibit increased resistance to metabolism and are thus useful for increasing the half-life of any compound described herein (e.g., compounds of Formula I, compounds of Formula A, or compounds of Formula B) when administered to a mammal, particularly a human. See, for example, Foster, “Deuterium Isotope Effects in Studies of Drug Metabolism”, Trends Pharmacol. Sci. 5(12):524-527 (1984). In view of the present disclosure, such compounds are synthesized by means known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I, the compound of Formula A, or the compound of Formula B.

In some embodiments, the carbon bonded to the 5 position on the tetrahydrofuranyl ring of Formula I is substituted with one or two deuterium atoms. In some embodiments, the compound of Formula I is

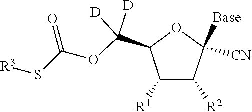

In some embodiments, the compound of Formula I is

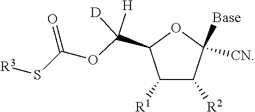

In some embodiments, a carbon of the Base of Formula I is substituted with one or more deuterium atoms. In some embodiments, Base is

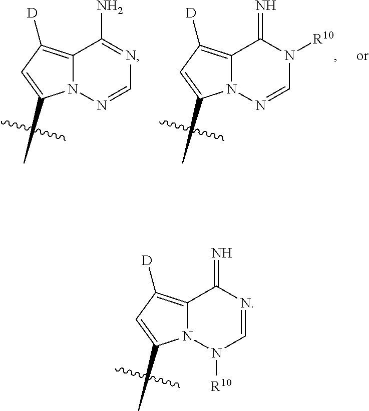

In some embodiments, a carbon on R¹⁰ of the Base of Formula I is substituted with one or more deuterium atoms. In some embodiments, a carbon on R¹ of Formula I is substituted with one or more deuterium atoms. In some embodiments, a carbon on R² of Formula I is substituted with one or more deuterium atoms. In some embodiments, a carbon on R³ of Formula I is substituted with one or more deuterium atoms.

In some embodiments, the compound of Formula I is

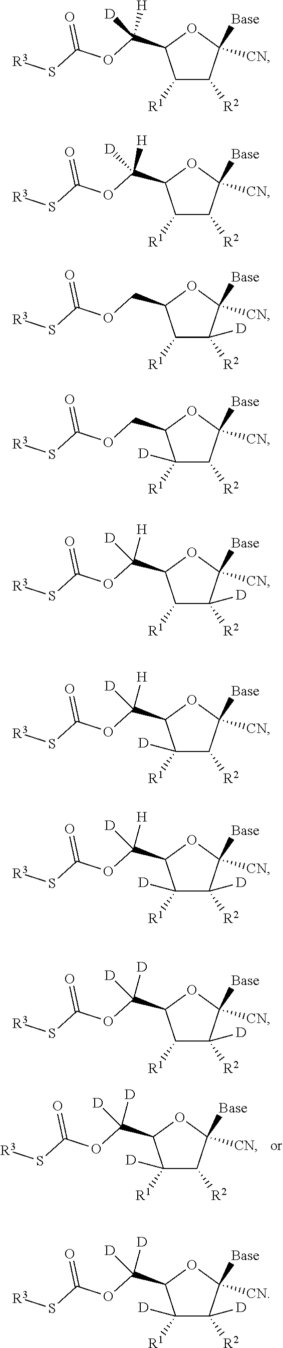

In some embodiments, Base is

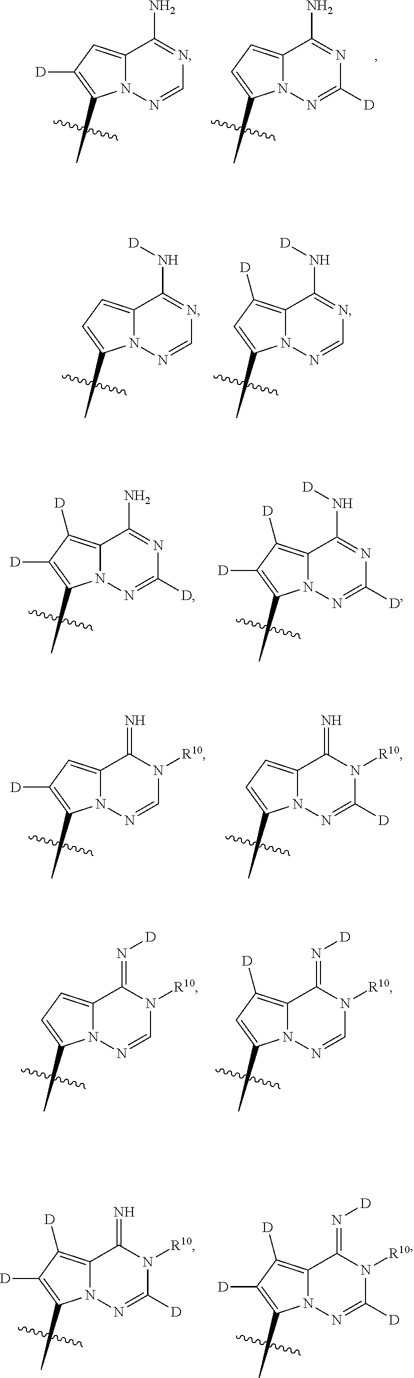

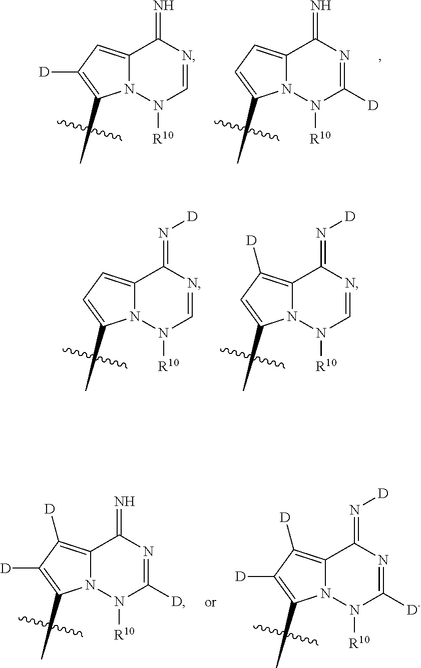

In some embodiments, the carbon bonded to the 5 position on the tetrahydrofuranyl ring of Formula A is substituted with one or two deuterium atoms. In some embodiments, the compound of Formula A is

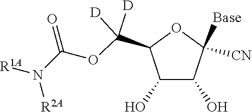

In some embodiments, the compound of Formula A is

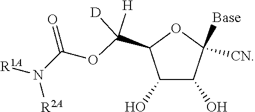

In some embodiments, a carbon of the Base of Formula A is substituted with one or more deuterium atoms. In some embodiments, Base is

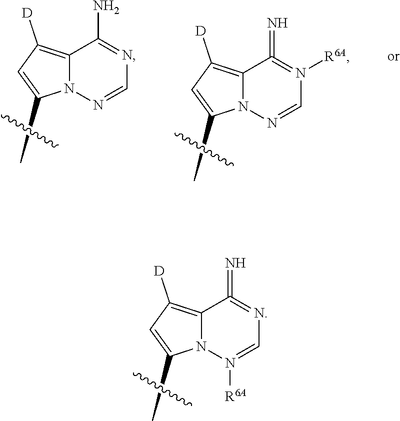

In some embodiments, a carbon on R^{6A} of the Base of Formula A is substituted with one or more deuterium atoms. In some embodiments, a carbon on R^{1A} of Formula A is substituted with one or more deuterium atoms. In some embodiments, a carbon on R^{2A} of Formula A is substituted with one or more deuterium atoms.

In some embodiments, the compound of Formula A is

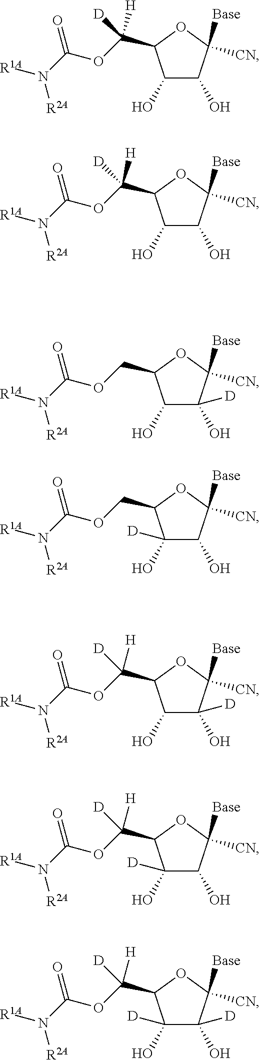

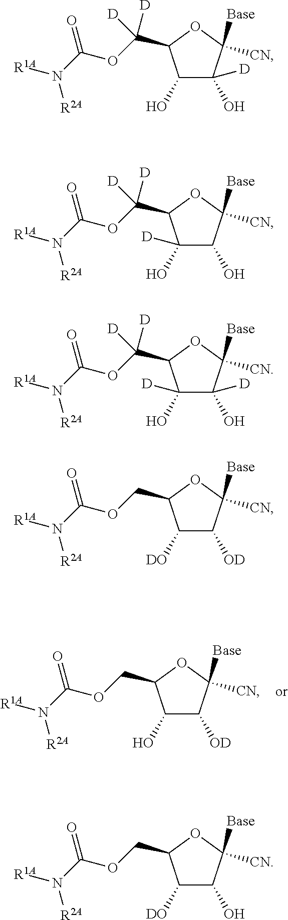

In some embodiments, Base is

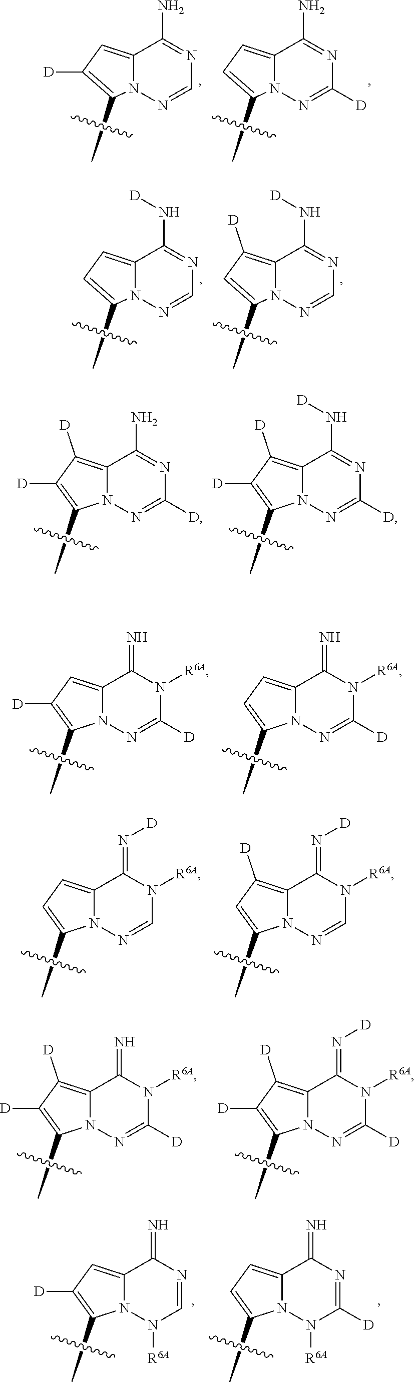

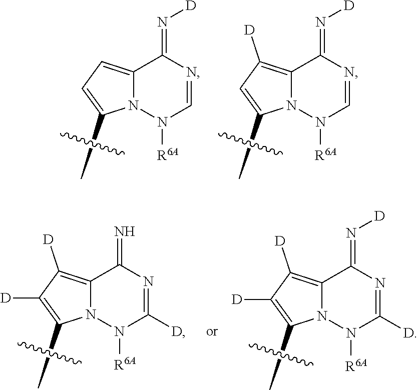

In some embodiments, the carbon bonded to the 5 position on the tetrahydrofuranyl ring of Formula B is substituted with one or two deuterium atoms. In some embodiments, the compound of Formula B is

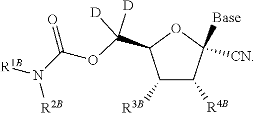

In some embodiments, the compound of Formula B is

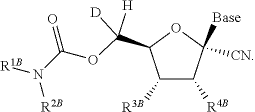

In some embodiments, a carbon of the Base of Formula B is substituted with one or more deuterium atoms. In some embodiments, Base is

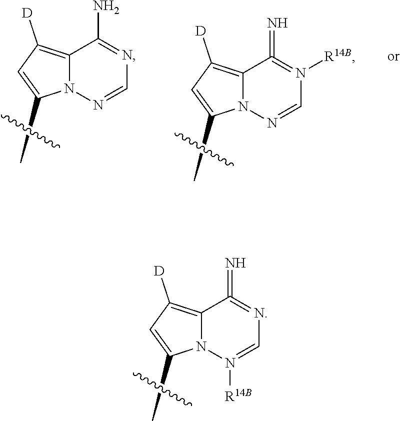

In some embodiments, a carbon on R^{14B} of the Base of Formula B is substituted with one or more deuterium atoms. In some embodiments, a carbon on R^{3B} of Formula B is substituted with one or more deuterium atoms. In some embodiments, a carbon on R^{4B} of Formula B is substituted with one or more deuterium atoms. In some embodiments, a carbon on R^{1B} of Formula B is substituted with one or more deuterium atoms. In some embodiments, a carbon on R^{2B} of Formula B is substituted with one or more deuterium atoms.

In some embodiments, the compound of Formula B is

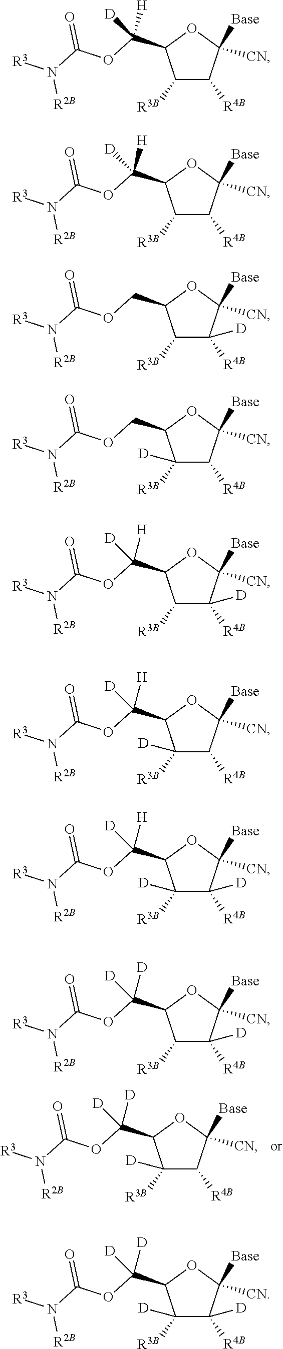

In some embodiments, Base is

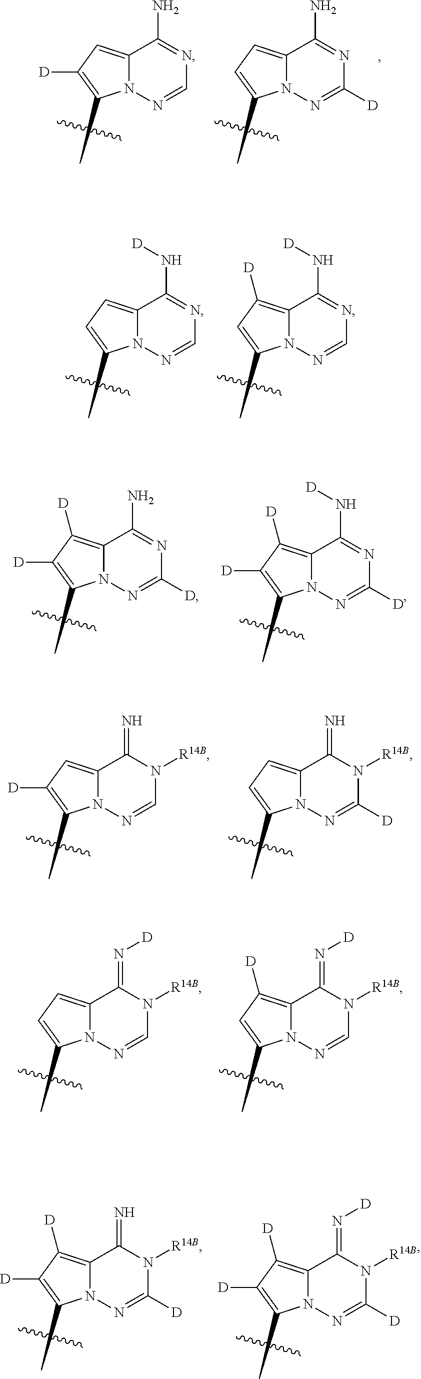

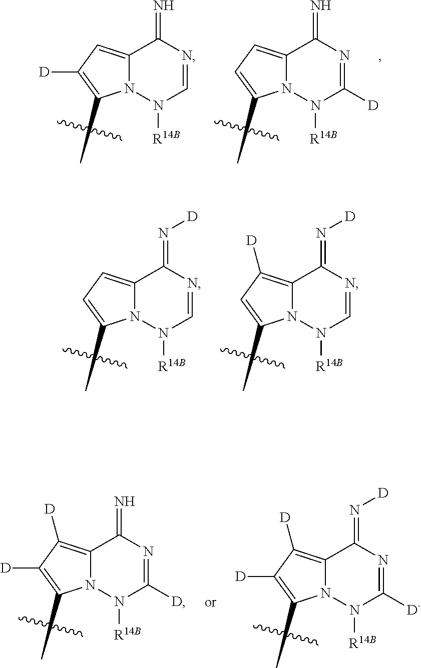

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Whenever a compound described herein is substituted with more than one of the same designated group, e.g., “R” or “R”, then it will be understood that the groups may be the same or different, i.e., each group is independently selected.

Wavy lines, [symbol] , indicate the site of covalent bond attachments to the adjoining substructures, groups, moieties, or atoms.

### III. Compounds

In certain embodiments, provided herein is a compound of Formula I:

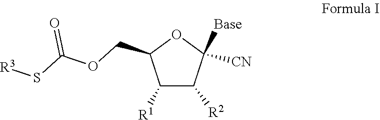

or a pharmaceutically acceptable salt thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O— or —OCR⁶O—;
R³, R⁴, and R⁵ are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R³, R⁴, and R⁵ are each, independently, optionally substituted with one, two or three substituents independently selected from the group consisting of halogen, cyano, —N₃, —OR⁷, —NR⁸R⁹, and phenyl optionally substituted with one, two or three substituents independently selected from halo, cyano, and C₁-C₆ alkyl;
R⁶ is H, C₁-C₆ alkyl or C₆-C₁₀ aryl;
each R⁷ is independently H, C₁-C₆ alkyl, C₁-C₆ haloalkyl, and C₃-C₆ cycloalkyl;
each R⁸ is independently H, C₁-C₆ alkyl, C₁-C₆ haloalkyl, and C₃-C₆ cycloalkyl;
each R⁹ is independently H, C₁-C₆ alkyl, C₁-C₆ haloalkyl, and C₃-C₆ cycloalkyl;
Base is

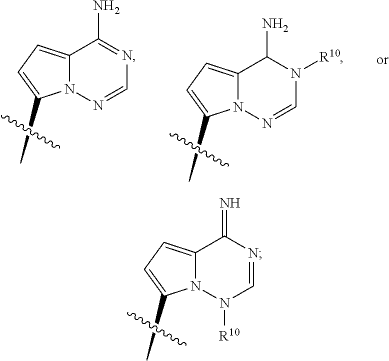

and R¹⁰ is C₁-C₆ alkyl substituted with —OP(O)(OH)₂.

In some embodiments, the compound has a Formula Ia:

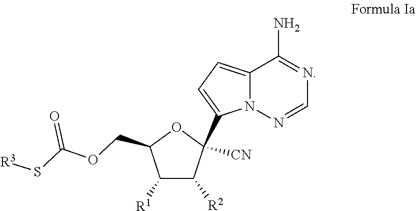

In some embodiments, R¹ is —OH. In some embodiments, R¹ is —OC(═O)R⁴. In some embodiments, R¹ is —OC(═O)OR⁴.

In some embodiments, R² is —OH. In some embodiments, R² is —OC(═O)R⁵. In some embodiments, R² is —OC(═O)OR⁵.

In some embodiments, R¹ is —OH and R² is —OH. In some embodiments, R¹ is —OC(═O)R⁴ and R² is —OC(═O)R⁵. In some embodiments, R¹ is —OC(═O)OR⁴ and R² is —OC(═O)OR⁵. In some embodiments, R¹ is —OH and R² is —OC(═O)R⁵. In some embodiments, R¹ is —OH and R² is —OC(═O)OR⁵. In some embodiments, R¹ is —OC(═O)R⁴ and R² is —OH. In some embodiments, R¹ is —OC(═O)OR⁴ and R² is —OH.

In some embodiments, R⁴ and R⁵ are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S.

In some embodiments, R⁴ and R⁵ are each independently C₁-C₈ alkyl or C₃-C₈ carbocyclyl.

In some embodiments, R⁴ and R⁵ are each independently C₁-C₈ alkyl.

In some embodiments, R⁴ and R⁵ are each independently C₁-C₃ alkyl.

In some embodiments, R⁴ and R⁵ are each methyl.

In some embodiments, R³ is C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S.

In some embodiments, R³ is C₁-C₈ alkyl, C₂-C₈ alkenyl, C₂-C₈ alkynyl, or C₃-C₈ carbocyclyl.

In some embodiments, R³ is C₁-C₈ alkyl or C₃-C₈ carbocyclyl. In some embodiments, R³ is methyl, ethyl, propyl, butyl, or cyclopentyl.

In some embodiments, R³ is C₃-C₈ carbocyclyl. In some embodiments, R³ is cyclopentyl.

In some embodiments, R³ is C₁-C₈ alkyl. In some embodiments, R³ is methyl, ethyl, propyl, or butyl.

In some embodiments, R³ is

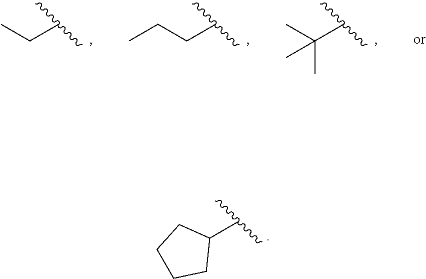

In some embodiments,
R¹ is —OH or —OC(═O)R⁴;
R² is —OH or —OC(═O)R⁵; and
R³, R⁴, and R⁵ are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S.

In some embodiments,
R¹ is —OH or —OC(═O)R⁴;
R² is —OH or —OC(═O)R⁵; and
R³, R⁴, and R⁵ are each independently C₁-C₈ alkyl, C₂-C₈ alkenyl, C₂-C₈ alkynyl, or C₃-C₈ carbocyclyl.

In some embodiments,
R¹ is —OH or —OC(═O)R⁴;
R² is —OH or —OC(═O)R⁵; and
R³, R⁴, and R⁵ are each independently C₁-C₈ alkyl or C₃-C₈ carbocyclyl.

In some embodiments,
R¹ is —OH or —OC(═O)R⁴;
R² is —OH or —OC(═O)R⁵;
R⁴ and R⁵ are each independently C₁-C₆ alkyl; and
R³ is C₁-C₈ alkyl or C₃-C₈ carbocyclyl.

In some embodiments,
R¹ is —OH or —OC(═O)R⁴;
R² is —OH or —OC(═O)R⁵;
R⁴ and R⁵ are each independently methyl or ethyl; and
R³ is C₁-C₆ alkyl or C₃-C₈ carbocyclyl.

In some embodiments, Base is

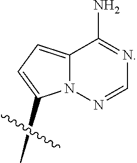

In some embodiments,
R² is —OH or —OC(═O)R⁴;
R² is —OH or —OC(═O)R⁵;
R³ is C₁-C₈ alkyl or C₃-C₈ carbocyclyl;
R⁴ and R⁵ are C₁-C₈ alkyl;
Base is

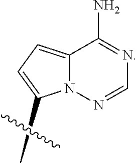

In some embodiments, the compound is selected from:

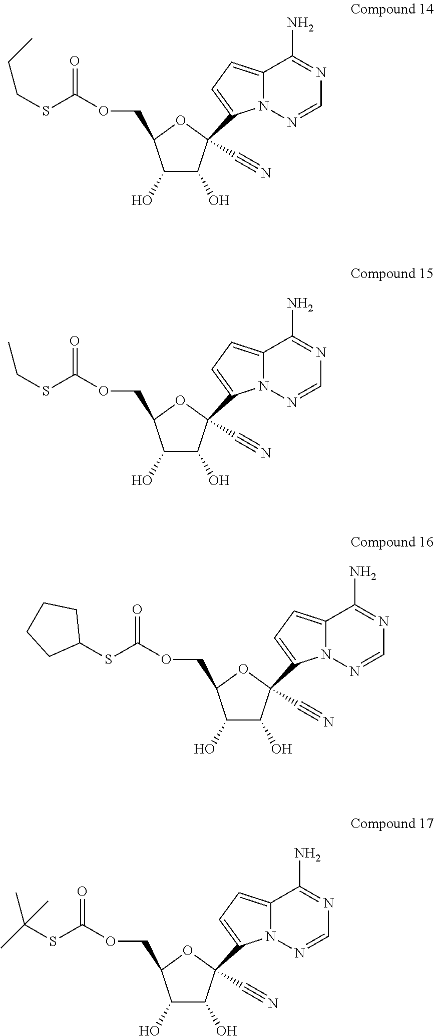 [SMILES: CCCSC(=O)OC[C@H]1O[C@@](C#N)(c2ccc3c(N)ncnn23)[C@H](O)[C@@H]1O ; CCSC(=O)OC[C@H]1O[C@@](C#N)(c2ccc3c(N)ncnn23)[C@H](O)[C@@H]1O ; N#C[C@@]1(c2ccc3c(N)ncnn23)O[C@H](COC(=O)SC2CCCC2)[C@@H](O)[C@H]1O ; CC(C)(C)SC(=O)OC[C@H]1O[C@@](C#N)(c2ccc3c(N)ncnn23)[C@H](O)[C@@H]1O]

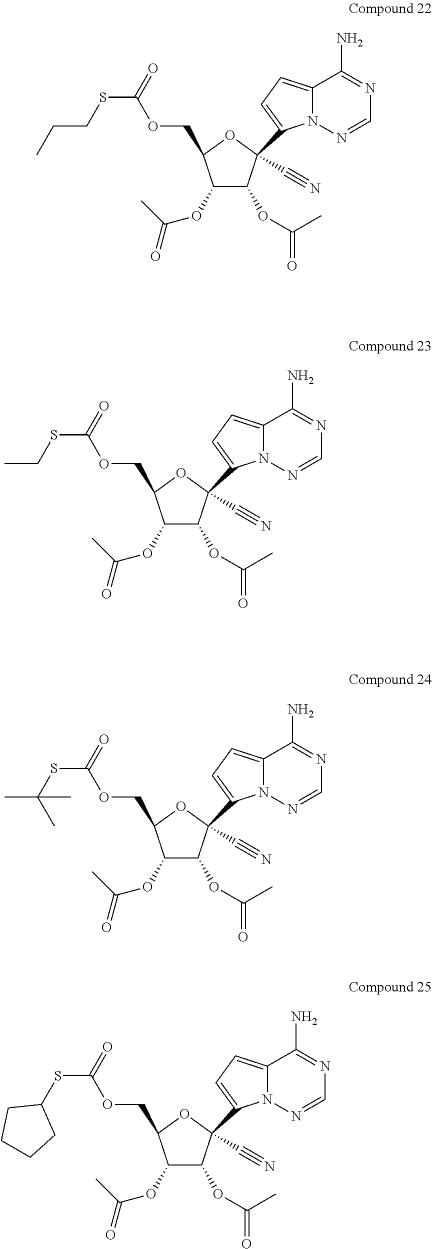 [SMILES: CCCSC(=O)OC[C@H]1O[C@@](C#N)(c2ccc3c(N)ncnn23)[C@H](OC(C)=O)[C@@H]1OC(C)=O ; CCSC(=O)OC[C@H]1O[C@@](C#N)(c2ccc3c(N)ncnn23)[C@H](OC(C)=O)[C@@H]1OC(C)=O ; CC(=O)O[C@H]1[C@@H](OC(C)=O)[C@](C#N)(c2ccc3c(N)ncnn23)O[C@@H]1COC(=O)SC(C)(C)C ; CC(=O)O[C@H]1[C@@H](OC(C)=O)[C@](C#N)(c2ccc3c(N)ncnn23)O[C@@H]1COC(=O)SC1CCCC1]

or a pharmaceutically acceptable salt thereof.

In certain embodiments, provided herein is a compound of Formula A:

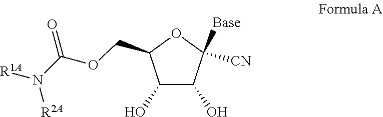

or a pharmaceutically acceptable salt thereof, wherein:
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
   wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —C(═O)R^{3A}, —C(═O)OR^{3A}, —NR^{4A}R^{5A}, C₁-C₆ alkyl, and phenyl;
      wherein the substituent C₁-C₆ alkyl of R^{1A} and R^{2A} is optionally substituted with one, two or three substituents independently selected from halo, cyano, and phenyl; and
      the substituent phenyl of R^{1A} and R^{2A} is optionally substituted with one, two or three substituents independently selected from halo, cyano, and C₁-C₆alkyl; or
R^{1A} is H;
R^{2A} is C₁-C₈ alkyl substituted with —C(═O)OR^{3A};
   wherein C₁-C₈ alkyl substituted with —C(═O)OR^{3A} of R^{2A} is optionally further substituted with one or two substituents independently selected from halo, cyano, and phenyl, and —OR^{3A};
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S;
each R^{4A} is independently H, C₁-C₆ alkyl, C₁-C₆ haloalkyl, and C₃-C₆ cycloalkyl;
each R^{5A} is independently H, C₁-C₆ alkyl, C₁-C₆ haloalkyl, and C₃-C₆ cycloalkyl;
Base is

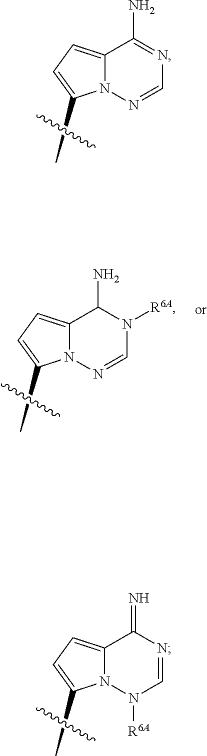

and
R^{6A} is C₁-C₆ alkyl substituted with —OP(O)(OH)₂.

In some embodiments, the compound is of Formula Aa:

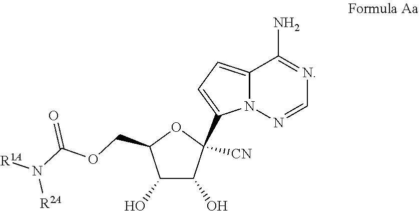

In some embodiments, R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —C(═O)R^{3A}, —C(═O)OR^{3A}, —NR^{4A}R^{5A}, C₁-C₆ alkyl, and phenyl;
   wherein the substituent C₁-C₆ alkyl of R^{1A} and R^{2A} is optionally substituted with one, two or three substituents independently selected from halo, cyano, and phenyl; and
   the substituent phenyl of R^{1A} and R^{2A} is optionally substituted with one, two or three substituents independently selected from halo, cyano, and C₁-C₆ alkyl.

In some embodiments, R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1A} and R^{2A} is optionally substituted with one, two or three substituents independently selected from the group consisting of oxo, —OR^{3A}, —C(═O)R^{3A}, —C(═O)OR^{3A}, C₁-C₆ alkyl, and phenyl;
   wherein the substituent C₁-C₆ alkyl of R^{1A} and R^{2A} is optionally substituted with one, two or three substituents independently selected from halo, cyano, and phenyl.

In some embodiments, R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1A} and R^{2A} is optionally substituted with one, two or three substituents independently selected from the group consisting of oxo, —OR^{3A}, —C(═O)OR^{3A}, and C₁-C₆ alkyl substituted with phenyl.

In some embodiments, R^{1A} and R^{2A} are joined to form 3 to 8 membered heterocyclyl containing 1, 2, or 3 heteroatoms selected independently from N, O, and S, 3 to 8 membered heterocyclyl containing 1, 2, or 3 heteroatoms selected independently from N, O, and S substituted with two oxo, 3 to 8 membered heterocyclyl containing 1, 2, or 3 heteroatoms selected independently from N, O, and S substituted with oxo and C₁-C₆ alkyl substituted with phenyl, or 3 to 8 membered heterocyclyl containing 1, 2, or 3 heteroatoms selected independently from N, O, and S substituted with —C(═O)OR^{3A}.

In some embodiments, R^{1A} and R^{2A} are joined to form

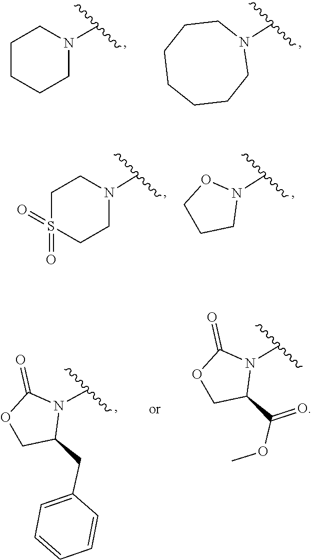 [SMILES: *N1CCCCC1 ; *N1CCCCCCC1 ; *N1CCS(=O)(=O)CC1 ; *N1CCCO1 ; *N1C(=O)OC[C@@H]1Cc1ccccc1 ; *N1C(=O)OC[C@@H]1C(=O)OC]

In some embodiments, R^{1A} and R^{2A} are joined to form 5 to 10 membered heteroaryl containing 1, 2, or 3 heteroatoms selected form N, O, and S substituted with —C(═O)OR^{3A}, 5 to 10 membered heteroaryl containing 1, 2, or 3 heteroatoms selected form N, O, and S substituted with —C(═O)OR^{3A} and —OR^{3A}, or 5 to 10 membered heteroaryl containing 1, 2, or 3 heteroatoms selected form N, O, and S substituted with —C(═O)OR^{3A}.

In some embodiments, R^{1A} and R^{2A} are joined to form

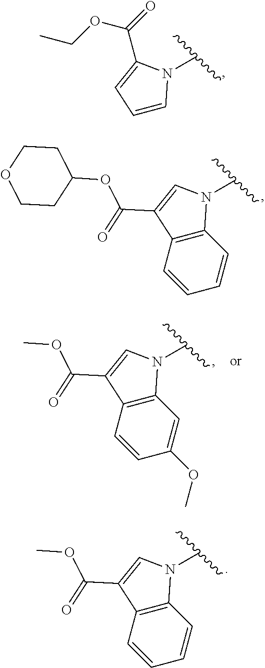 [SMILES: *n1cccc1C(=O)OCC ; *n1cc(C(=O)OC2CCOCC2)c2ccccc21 ; *n1cc(C(=O)OC)c2ccc(OC)cc21 ; *n1cc(C(=O)OC)c2ccccc21]

In some embodiments, R^{1A} is H and R^{2A} is C₁-C₈ alkyl substituted with —C(═O)OR^{3A}; wherein C₁-C₈ alkyl substituted with —C(═O)OR^{3A} of R^{2A} is optionally further substituted with one or two substituents independently selected from halo, cyano, and phenyl, and —OR^{3A}.

In some embodiments, R^{2A} is C₁-C₈ alkyl substituted with —C(═O)OR^{3A}. In some embodiments, R^{2A} is C₁-C₈ alkyl substituted with —C(═O)OH or —C(═O)O(CH)(CH₃)₂.

In some embodiments, R^{2A} is

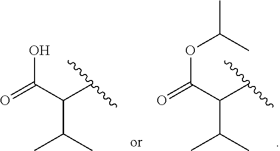

In some embodiments, R^{2A} is

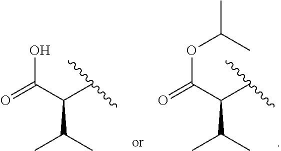

In some embodiments, R^{3A} is H, C₁-C₆ alkyl, or 3 to 8 membered heterocyclyl containing 1, 2, or 3 heteroatoms selected independently from N, O, and S. In some embodiments, R^{3A} is H. In some embodiments, R^{3A} is C₁-C₆ alkyl. In some embodiments, R^{3A} is 3 to 8 membered heterocyclyl containing 1, 2, or 3 heteroatoms selected independently from N, O, and S. In some embodiments, R^{3A} is

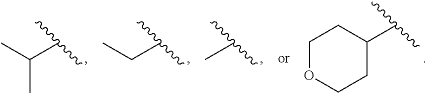

In some embodiments,
R^{1A} is H;
R^{2A} is C₁-C₈ alkyl substituted with COOR^{3A};
   wherein C₁-C₈ alkyl substituted with —C(═O)OR^{3A} of R^{2A} is optionally further substituted with one or two substituents independently selected from halo, cyano, phenyl, and —OR^{3A}; and
each R^{3A} is independently H, C₁-C₆ alkyl, C₁-C₆ haloalkyl, C₃-C₆ cycloalkyl, or 3 to 8 membered heterocyclyl containing 1, 2, or 3 heteroatoms selected independently from N, O, and S.

In some embodiments,
R^{1A} is H;
R^{2A} is C₁-C₈ alkyl substituted with —C(═O)OR^{3A}; and
R^{3A} is H, C₁-C₆ alkyl, C₁-C₆ haloalkyl, C₃-C₆ cycloalkyl or 3 to 8 membered heterocyclyl containing 1, 2, or 3 heteroatoms selected independently from N, O, and S.

In some embodiments,
R^{1A} is H;
R^{2A} is C₁-C₈ alkyl substituted with —C(═O)OR^{3A}; and
R^{3A} is H or C₁-C₆ alkyl.

In some embodiments,
R^{1A} is H;
R^{2A} is C₁-C₆ alkyl substituted with —C(═O)OR^{3A}; and
R^{3A} is H or C₁-C₃ alkyl.

In some embodiments, the compound is:

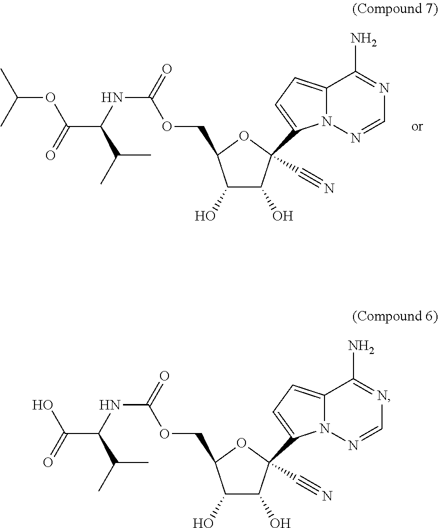 [SMILES: CC(C)OC(=O)[C@@H](NC(=O)OC[C@H]1O[C@@](C#N)(c2ccc3c(N)ncnn23)[C@H](O)[C@@H]1O)C(C)C ; CC(C)[C@H](NC(=O)OC[C@H]1O[C@@](C#N)(c2ccc3c(N)ncnn23)[C@H](O)[C@@H]1O)C(=O)O]

or a pharmaceutically acceptable salt thereof.

In some embodiments,
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
   wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —COR^{3A}, —COOR^{3A}, —NR^{4A}R^{5A}, C₁-C₆ alkyl, and phenyl;
      wherein the substituent C₁-C₆ alkyl of R^{1A} and R^{2A} is optionally substituted with one, two or three substituents independently selected from halo, cyano, and phenyl; and
      the substituent phenyl of R^{1A} and R^{2A} is optionally substituted with one, two or three substituents independently selected from halo, cyano, and C₁-C₆ alkyl;
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S;
each R^{4A} is independently H, C₁-C₆ alkyl, C₁-C₆ haloalkyl, and C₃-C₆ cycloalkyl; and
each R^{5A} is independently H, C₁-C₆ alkyl, C₁-C₆ haloalkyl, and C₃-C₆ cycloalkyl.

In some embodiments,
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
   wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —OR^{3A}, —C(═O)R^{3A}, —C(═O)OR^{3A}, C₁-C₆ alkyl, and phenyl;
      wherein the substituent C₁-C₆ alkyl of R^{1A} and R^{2A} is optionally substituted with one, two or three substituents independently selected from halo, cyano, and phenyl; and
      the substituent phenyl of R^{1A} and R^{2A} is optionally substituted with one, two or three substituents independently selected from halo, cyano, and C₁-C₆alkyl; and
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S.

In some embodiments, Base is

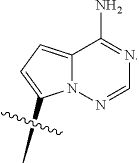

In some embodiments,
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
   wherein the 3 to 8 membered heterocyclyl or 5 to 10 membered heteroaryl of R^{1A} and R^{2A} is optionally substituted with one or two substituents independently selected from the group consisting of oxo, —OR^{3A}, —C(═O)OR^{3A}, and C₁-C₆ alkyl;
      wherein the substituent C₁-C₆ alkyl of R^{1A} and R^{2A} is optionally substituted phenyl;
R^{1A} is H;
R^{2A} is C₁-C₈ alkyl substituted with —C(═O)OR^{3A};
each R^{3A} is independently H or C₁-C₆ alkyl; and
Base is

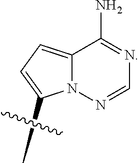

In some embodiments, the compound is selected from:

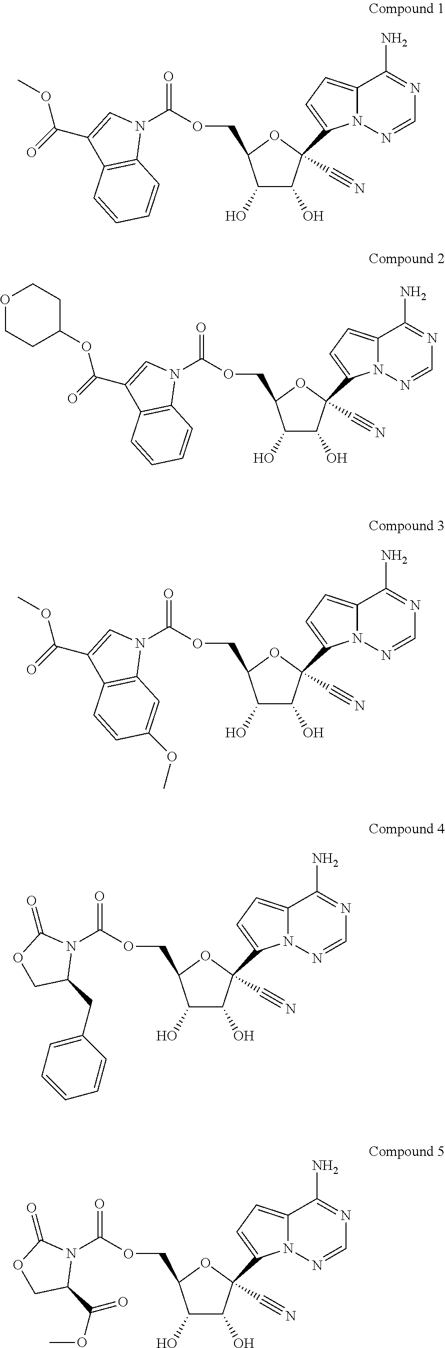 [SMILES: COC(=O)c1cn(C(=O)OC[C@H]2O[C@@](C#N)(c3ccc4c(N)ncnn34)[C@H](O)[C@@H]2O)c2ccccc12 ; N#C[C@@]1(c2ccc3c(N)ncnn23)O[C@H](COC(=O)n2cc(C(=O)OC3CCOCC3)c3ccccc32)[C@@H](O)[C@H]1O ; COC(=O)c1cn(C(=O)OC[C@H]2O[C@@](C#N)(c3ccc4c(N)ncnn34)[C@H](O)[C@@H]2O)c2cc(OC)ccc12 ; N#C[C@@]1(c2ccc3c(N)ncnn23)O[C@H](COC(=O)N2C(=O)OC[C@@H]2Cc2ccccc2)[C@@H](O)[C@H]1O ; COC(=O)[C@H]1COC(=O)N1C(=O)OC[C@H]1O[C@@](C#N)(c2ccc3c(N)ncnn23)[C@H](O)[C@@H]1O]

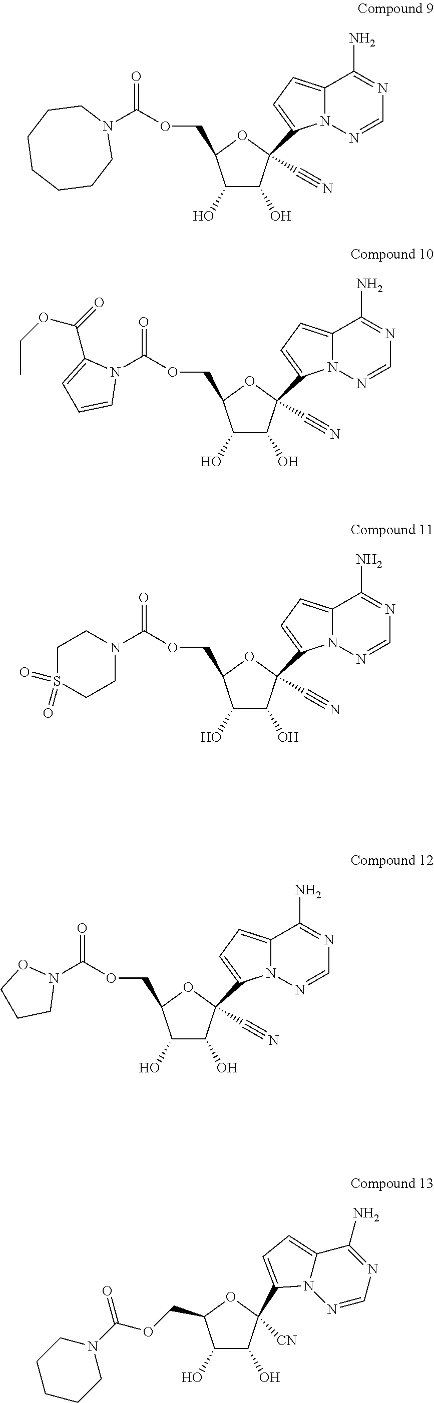 [SMILES: N#C[C@@]1(c2ccc3c(N)ncnn23)O[C@H](COC(=O)N2CCCCCCC2)[C@@H](O)[C@H]1O ; CCOC(=O)c1cccn1C(=O)OC[C@H]1O[C@@](C#N)(c2ccc3c(N)ncnn23)[C@H](O)[C@@H]1O ; N#C[C@@]1(c2ccc3c(N)ncnn23)O[C@H](COC(=O)N2CCS(=O)(=O)CC2)[C@@H](O)[C@H]1O ; N#C[C@@]1(c2ccc3c(N)ncnn23)O[C@H](COC(=O)N2CCCO2)[C@@H](O)[C@H]1O ; N#C[C@@]1(c2ccc3c(N)ncnn23)O[C@H](COC(=O)N2CCCCC2)[C@@H](O)[C@H]1O]

or a pharmaceutically acceptable salt thereof.

In certain embodiments, provided herein is a compound of Formula B:

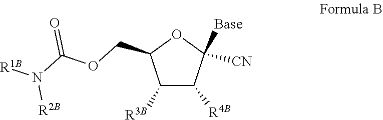

or a pharmaceutically acceptable salt thereof, wherein:
R^{3B} is —OC(═O)R^{5B} or —OC(═O)OR^{5B};
R^{4B} is —OC(═O)R^{6B} or —OC(═O)OR^{6B}; or
R^{3B} and R^{4B} are taken together to form —OC(═O)O— or —OCHR^{7B}O—;
R^{5B} and R^{6B} are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{5B} and R^{6B} are each, independently, optionally substituted with one, two or three substituents independently selected from the group consisting of halogen, cyano, —N₃, —OR^{8B}, —NR^{9B}R^{10B}, and phenyl optionally substituted with one, two or three substituents independently selected from halo, cyano, and C₁-C₆ alkyl;
each R^{8B} is independently H, C₁-C₆ alkyl, C₁-C₆ haloalkyl, and C₃-C₆ cycloalkyl;
each R^{9B} is independently H, C₁-C₆ alkyl, C₁-C₆ haloalkyl, and C₃-C₆ cycloalkyl;
each R^{10B} is independently H, C₁-C₆ alkyl, C₁-C₆ haloalkyl, and C₃-C₆ cycloalkyl;
R^{7B} is H, C₁-C₆ alkyl or C₆-C₁₀ aryl;
R^{1B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{1B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl;
R^{2B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{2B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of halogen, cyano, oxo, —N₃, —OR^{11B}, —C(═O)R^{11B}, —C(═O)OR^{11B}, —NR^{12B}R^{13B}, C₁-C₆ alkyl, and phenyl;
      wherein the substituent C₁-C₆ alkyl of R^{1B} and R^{2B} is optionally substituted with one, two or three substituents independently selected from halo, cyano, and phenyl; and
      the substituent phenyl of R^{1B} and R^{2B} is optionally substituted with one, two or three substituents independently selected from halo, cyano, and C₁-C₆ alkyl;
each R^{11B} is independently H, C₁-C₆ alkyl, C₁-C₆ haloalkyl, C₃-C₆ cycloalkyl and 3 to 8 membered heterocyclyl containing 1, 2, or 3 heteroatoms selected independently from N, O, and S;
each R^{12B} is independently H, C₁-C₆ alkyl, C₁-C₆ haloalkyl, and C₃-C₆ cycloalkyl;
each R^{13B} is independently H, C₁-C₆ alkyl, C₁-C₆ haloalkyl, and C₃-C₆ cycloalkyl;
Base is

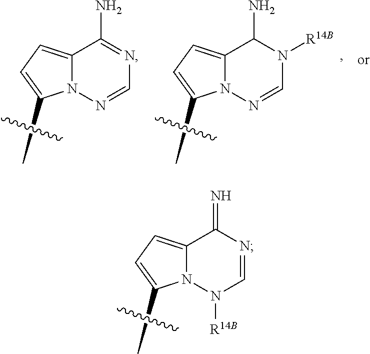

and
R^{14B} is C₁-C₆ alkyl substituted with —OP(O)(OH)₂.

In some embodiments, R^{3B} is —OC(═O)R^{5B} or —OC(═O)OR^{5B} and R^{4B} is —OC(═O)R^{6B} or —OC(═O)OR^{6B}. In some embodiments, R^{3B} is —OC(═O)R^{5B}. In some embodiments, R^{3B} is —OC(═O)OR^{5B}. In some embodiments, R^{4B} is —OC(═O)R^{6B}. In some embodiments, R^{4B} is —OC(═O)OR^{6B}. In some embodiments, R^{3B} is —OC(═O)R^{5B} and R^{4B} is —OC(═O)R^{6B}. In some embodiments, R^{3B} is —OC(═O)OR^{5B} and R^{4B} is —OC(═O)OR^{6B}. In some embodiments, R^{3B} is —OC(═O)R^{5B} and R^{4B} is —OC(═O)OR^{6B}. In some embodiments, R^{3B} is —OC(═O)OR^{5B} and R^{4B} is —OC(═O)R^{6B}.

In some embodiments, R^{3B} and R^{4B} are taken together to form —OC(═O)O— or —OCHR^{7B}O—. In some embodiments, R^{3B} and R^{4B} are taken together to form —OC(═O)O—. In some embodiments, R^{3B} and R^{4B} are taken together to form —OCHR^{7B}O—.

In some embodiments, the compound is of Formula Ba:

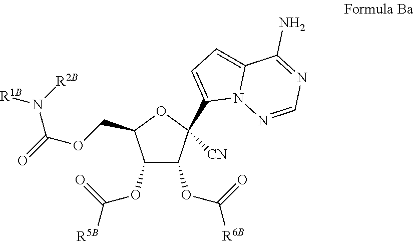

In some embodiments, R^{5B} and R^{6B} are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S. In some embodiments, R^{5B} and R^{6B} are each independently C₁-C₈ alkyl, C₂-C₈ alkenyl, C₂-C₈ alkynyl, or C₃-C₈ carbocyclyl. In some embodiments, R^{5B} and R^{6B} are each independently C₁-C₈ alkyl. In some embodiments, R^{5B} and R^{6B} are each independently C₁-C₆ alkyl. In some embodiments, R^{5B} and R^{6B} are each independently C₁-C₃ alkyl. In some embodiments, R^{5B} and R^{6B} are each —CH₃.

In some embodiments, R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of halogen, cyano, oxo, —N₃, —OR^{11B}, —C(═O)R^{11B}, —C(═O)OR^{11B}, —NR^{12B}R^{13B}, C₁-C₆ alkyl, and phenyl;
   wherein the substituent C₁-C₆ alkyl of R^{1B} and R^{2B} is optionally substituted with one, two or three substituents independently selected from halo, cyano, and phenyl; and
   the substituent phenyl of R^{1B} and R^{2B} is optionally substituted with one, two or three substituents independently selected from halo, cyano, and C₁-C₆ alkyl.

In some embodiments, R^{1B} and R^{2B} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)R^{11B}, —C(═O)OR^{11B}, C₁-C₆ alkyl, and phenyl;
   wherein the substituent C₁-C₆ alkyl of R^{1A} and R^{2A} is optionally substituted with one, two or three substituents independently selected from halo, cyano, and phenyl.

In some embodiments, R^{1B} and R^{2B} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, and C₁-C₆ alkyl substituted with phenyl.

In some embodiments, R^{1B} and R^{2B} are joined to form 3 to 8 membered heterocyclyl containing 1, 2, or 3 heteroatoms selected independently from N, O, and S, 3 to 8 membered heterocyclyl containing 1, 2, or 3 heteroatoms selected independently from N, O, and S substituted with two oxo, 3 to 8 membered heterocyclyl containing 1, 2, or 3 heteroatoms selected independently from N, O, and S substituted with oxo and C₁-C₆ alkyl substituted with phenyl, or 3 to 8 membered heterocyclyl containing 1, 2, or 3 heteroatoms selected independently from N, O, and S substituted with —C(═O)OR^{11B}.

In some embodiments, R^{1B} and R^{2B} are joined to form

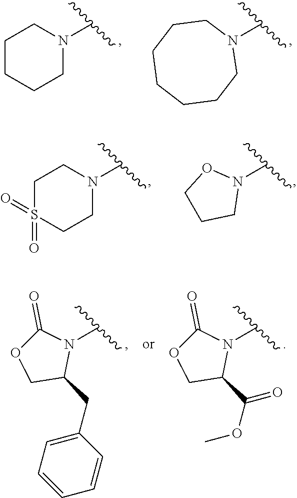 [SMILES: *N1CCCCC1 ; *N1CCCCCCC1 ; *N1CCS(=O)(=O)CC1 ; *N1CCCO1 ; *N1C(=O)OC[C@@H]1Cc1ccccc1 ; *N1C(=O)OC[C@@H]1C(=O)OC]

In some embodiments, R^{1B} and R^{2B} are joined to form 5 to 10 membered heteroaryl containing 1, 2, or 3 heteroatoms selected form N, O, and S substituted with —C(═O)OR^{3A}, 5 to 10 membered heteroaryl containing 1, 2, or 3 heteroatoms selected form N, O, and S substituted with —C(═O)OR^{3A} and —OR^{3A}, or 5 to 10 membered heteroaryl containing 1, 2, or 3 heteroatoms selected form N, O, and S substituted with —C(═O)OR^{3A}.

In some embodiments, R^{1B} and R^{2B} are joined to form

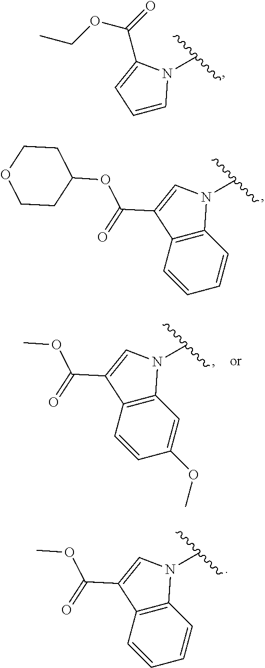 [SMILES: *n1cccc1C(=O)OCC ; *n1cc(C(=O)OC2CCOCC2)c2ccccc21 ; *n1cc(C(=O)OC)c2ccc(OC)cc21 ; *n1cc(C(=O)OC)c2ccccc21]

In some embodiments, R^{1B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{1B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl; and
R^{2B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{2B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl.

In some embodiments, R^{1B} is H, C₁-C₈ alkyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₆-C₁₀ aryl, or 5 to 6 membered heteroaryl of R^{1B} is optionally substituted with —C(═O)OR^{11B}.

In some embodiments, R^{1B} is C₁-C₈ alkyl. In some embodiments, R^{1B} is

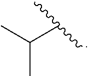 [SMILES: *C(C)C]

In some embodiments, R^{1B} is C₁-C₈ alkyl substituted with —C(═O)OR^{11B}. In some embodiments, R^{1B} is

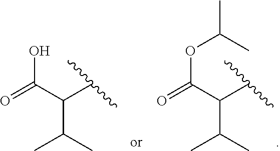

In some embodiments, R^{1B} is

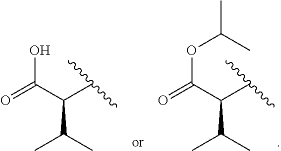

In some embodiments, R^{1B} is

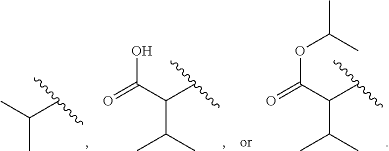

In some embodiments, R^{1B} is H.

In some embodiments, R^{2B} is C₁-C₈ alkyl. In some embodiments, R^{2B} is

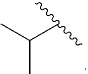 [SMILES: *C(C)C]

In some embodiments, R^{2B} is C₁-C₈ alkyl substituted with —C(═O)OR^{11B}. In some embodiments, R^{1B} is

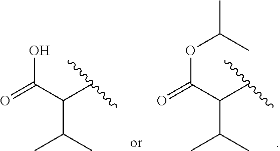

In some embodiments, R^{2B} is

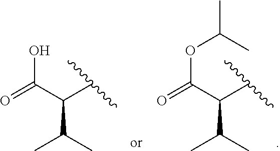

In some embodiments, R^{2B} is

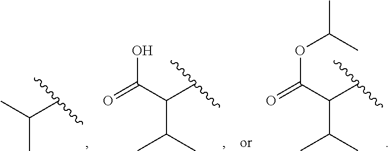

In some embodiments, R^{2B} is H.

In some embodiments, R^{11B} is H, C₁-C₆ alkyl, or 3 to 8 membered heterocyclyl containing 1, 2, or 3 heteroatoms selected independently from N, O, and S. In some embodiments, R^{11B} is H. In some embodiments, R^{11B} is C₁-C₆ alkyl. In some embodiments, R^{11B} is 3 to 8 membered heterocyclyl containing 1, 2, or 3 heteroatoms selected independently from N, O, and S. In some embodiments, R^{11B} is

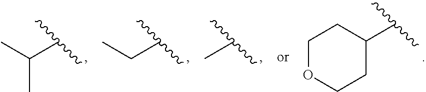

In some embodiments,
R^{1B} is H;
R^{2B} is C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{2B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of halogen, cyano, oxo, —N₃, —OR^{11B}, —C(═O)R^{11B}, —C(═O)OR^{11B}, —NR^{12B}R^{13B}, C₁-C₆ alkyl, and phenyl;
      wherein the substituent C₁-C₆ alkyl of R^{1B} and R^{2B} is optionally substituted with one, two or three substituents independently selected from halo, cyano, and phenyl; and
      the substituent phenyl of R^{1B} and R^{2B} is optionally substituted with one, two or three substituents independently selected from halo, cyano, and C₁-C₆alkyl; and
each R^{11B} is independently H, C₁-C₆ alkyl, C₁-C₆ haloalkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₈ alkyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₃-C₈ carbocyclyl, C₆-C₁₀ aryl, or 5 to 6 membered heteroaryl of R^{2B} is optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —ORB, —C(═O)OR^{11B}, C₁-C₆ alkyl, and phenyl;
      wherein the substituent C₁-C₆ alkyl of R^{1B} and R^{2B} is optionally substituted with one, two or three substituents independently selected from halo, cyano; and phenyl; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₈ alkyl, C₃-C₈ carbocyclyl, C₆-C₁₀ aryl, or 5 to 6 membered heteroaryl containing 1, 2, or 3 heteroatoms selected form N, O, and S;
   wherein C₁-C₈ alkyl, C₃-C₈ carbocyclyl, C₆-C₁₀ aryl, or 5 to 6 membered heteroaryl of R^{2B} is optionally substituted with one, two or three substituents independently selected from the group consisting of halogen, —OR^{11B}, and —C(═O)OR^{11B}; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

In some embodiments,
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, C₁-C₆ alkyl, and phenyl;
      wherein the substituent C₁-C₆ alkyl of R^{1B} and R^{2B} is optionally substituted with one, two or three substituents independently selected from halo, cyano; and phenyl; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₈ alkyl or C₃-C₈ carbocyclyl;
   wherein C₁-C₈ alkyl or C₃-C₈ carbocyclyl of R^{2B} is optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —ORB, —C(═O)OR^{11B}, and C₁-C₆ alkyl;
      wherein the substituent C₁-C₆ alkyl of R^{1B} and R^{2B} is optionally substituted with one, two or three substituents independently selected from halo, cyano; and phenyl; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₈ alkyl optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, and C₁-C₆ alkyl;
      wherein the substituent C₁-C₆ alkyl of R^{1B} and R^{2B} is optionally substituted with one, two or three substituents independently selected from halo, cyano; and phenyl; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₆ alkyl or C₃-C₈ carbocyclyl;
   wherein C₁-C₆ alkyl or C₃-C₈ carbocyclyl of R^{2B} is optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —ORB, —C(═O)OR^{11B}, and C₁-C₃ alkyl;
      wherein the substituent C₁-C₃ alkyl of R^{1B} and R^{2B} is optionally substituted with one, two or three substituents independently selected from halo, cyano; and phenyl; and
each R^{11B} is independently H, C₁-C₃ alkyl, C₃-C₆ cycloalkyl and 3 to 8 membered heterocyclyl containing 1, 2, or 3 heteroatoms selected independently from N, O, and S.

In some embodiments,
R^{1B} is H;
R^{2B} is C₁-C₆ alkyl optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, and C₁-C₃ alkyl;
      wherein the substituent C₁-C₃ alkyl of R^{1B} and R^{2B} is optionally substituted with one, two or three substituents independently selected from halo, cyano; and phenyl; and
each R^{11B} is independently H, C₁-C₃ alkyl, C₃-C₆ cycloalkyl and 3 to 8 membered heterocyclyl containing 1, 2, or 3 heteroatoms selected independently from N, O, and S.

In some embodiments, Base is

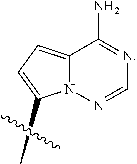

In some embodiments
R^{3B} is —OC(═O)R^{5B};
R^{4B} is —OC(═O)R^{6B};
R^{5B} and R^{6B} are each C₁-C₈ alkyl;
R^{1B} is H;
R^{2B} is C₁-C₈ alkyl optionally substituted with —C(═O)R^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
   wherein the 3 to 8 membered heterocyclyl or 5 to 10 membered heteroaryl of R^{1B} and R^{2B} is optionally substituted with one or two substituents independently selected from the group consisting of oxo, —OR^{11B}, —C(═O)OR^{11B} and C₁-C₆ alkyl;
      wherein the substituent C₁-C₆ alkyl of R^{1B} and R^{2B} is optionally substituted with phenyl;
each R^{11B} is independently H, C₁-C₆ alkyl, and 3 to 8 membered heterocyclyl containing 1, 2, or 3 heteroatoms selected independently from N, O, and S;
Base is

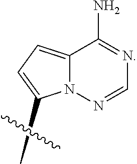

In some embodiments, the compound is selected from:

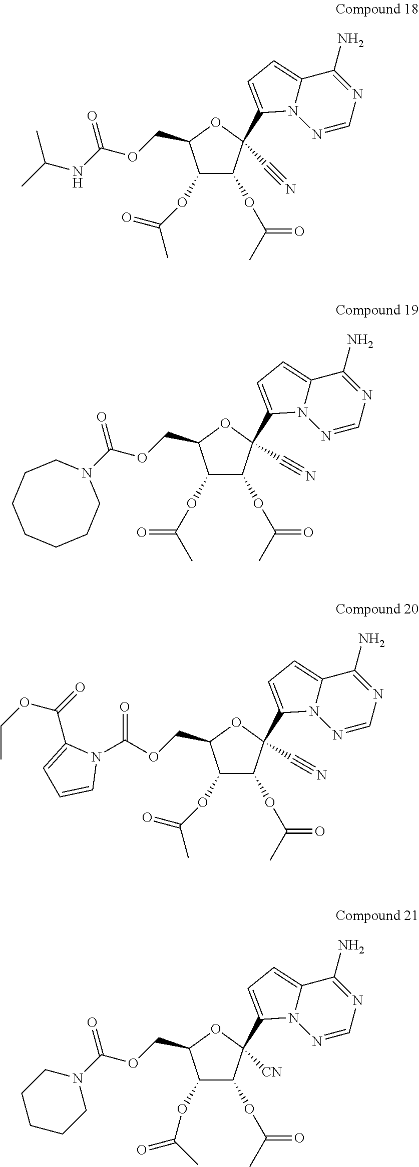 [SMILES: CC(=O)O[C@H]1[C@@H](OC(C)=O)[C@](C#N)(c2ccc3c(N)ncnn23)O[C@@H]1COC(=O)NC(C)C ; CC(=O)O[C@H]1[C@@H](OC(C)=O)[C@](C#N)(c2ccc3c(N)ncnn23)O[C@@H]1COC(=O)N1CCCCCCC1 ; CCOC(=O)c1cccn1C(=O)OC[C@H]1O[C@@](C#N)(c2ccc3c(N)ncnn23)[C@H](OC(C)=O)[C@@H]1OC(C)=O ; CC(=O)O[C@H]1[C@@H](OC(C)=O)[C@](C#N)(c2ccc3c(N)ncnn23)O[C@@H]1COC(=O)N1CCCCC1]

or a pharmaceutically acceptable salt thereof.

In some embodiments, the compound is selected from:

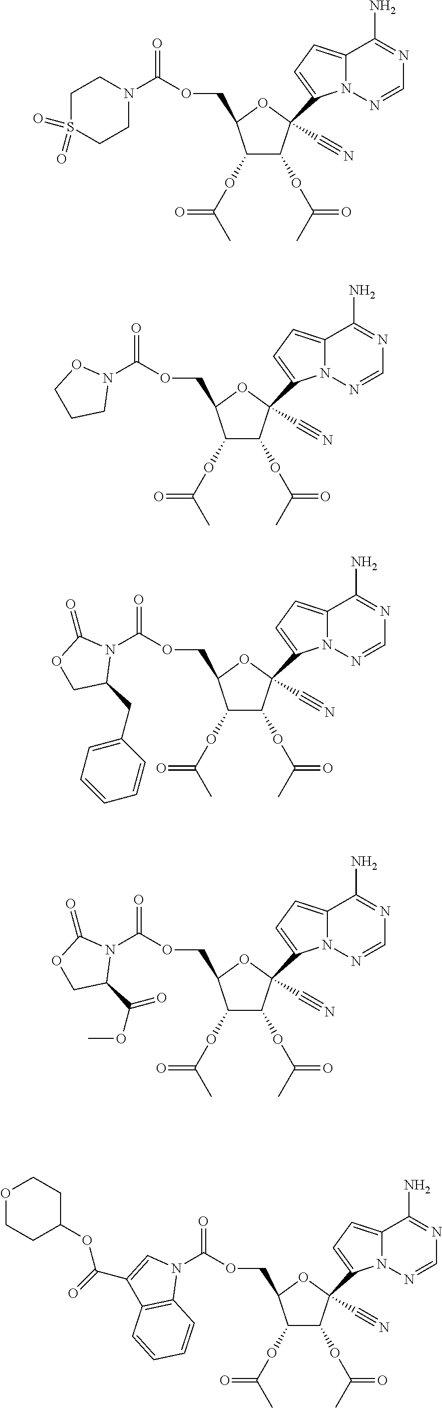 [SMILES: CC(=O)O[C@H]1[C@@H](OC(C)=O)[C@](C#N)(c2ccc3c(N)ncnn23)O[C@@H]1COC(=O)N1CCS(=O)(=O)CC1 ; CC(=O)O[C@H]1[C@@H](OC(C)=O)[C@](C#N)(c2ccc3c(N)ncnn23)O[C@@H]1COC(=O)N1CCCO1 ; CC(=O)O[C@H]1[C@@H](OC(C)=O)[C@](C#N)(c2ccc3c(N)ncnn23)O[C@@H]1COC(=O)N1C(=O)OC[C@@H]1Cc1ccccc1 ; COC(=O)[C@H]1COC(=O)N1C(=O)OC[C@H]1O[C@@](C#N)(c2ccc3c(N)ncnn23)[C@H](OC(C)=O)[C@@H]1OC(C)=O ; CC(=O)O[C@H]1[C@@H](OC(C)=O)[C@](C#N)(c2ccc3c(N)ncnn23)O[C@@H]1COC(=O)n1cc(C(=O)OC2CCOCC2)c2ccccc21]

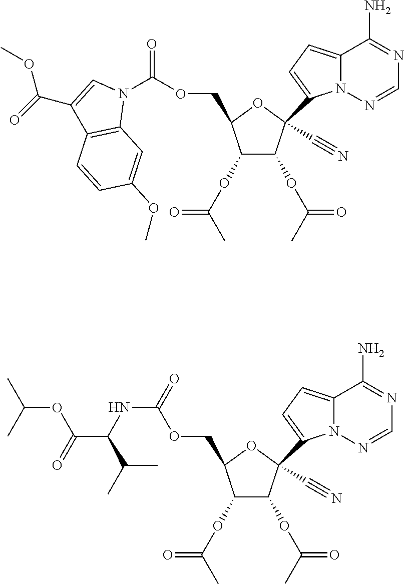 [SMILES: COC(=O)c1cn(C(=O)OC[C@H]2O[C@@](C#N)(c3ccc4c(N)ncnn34)[C@H](OC(C)=O)[C@@H]2OC(C)=O)c2cc(OC)ccc12 ; CC(=O)O[C@H]1[C@@H](OC(C)=O)[C@](C#N)(c2ccc3c(N)ncnn23)O[C@@H]1COC(=O)N[C@H](C(=O)OC(C)C)C(C)C]

or a pharmaceutically acceptable salt thereof.

### IV. Pharmaceutical Formulations

The compounds disclosed herein may be formulated with conventional carriers and excipients. For example, tablets will contain excipients, glidants, fillers, binders and the like. Aqueous formulations are prepared in sterile form, and when intended for delivery by other than oral administration generally will be isotonic. All formulations may optionally comprise excipients such as those set forth in the “Handbook of Pharmaceutical Excipients” (1986). Pharmaceutically acceptable excipients include ascorbic acid and other antioxidants, chelating agents such as EDTA, carbohydrates such as dextran, hydroxyalkylcellulose, hydroxyalkylmethylcellulose, stearic acid and the like. In some embodiments, the formulations comprise one or more pharmaceutically acceptable excipients. The pH of the formulations ranges from about 3 to about 11, but is ordinarily about 7 to 10. In some embodiments, the pH of the formulations ranges from about 2 to about 5, but is ordinarily about 3 to 4.

While it is possible for the compounds of the disclosure (“the active ingredients”) to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any appropriate method known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In some embodiments, the pharmaceutical formulation is for subcutaneous, intramuscular, intravenous, oral, or inhalation administration.

In some embodiments, the compound described herein e.g., the compound of Formula I, compound of Formula A, or compound of Formula B, or the pharmaceutically acceptable salt thereof, described herein have optimized/improved pharmacokinetic properties and are amenable to oral administration. For example, the compounds of Formula I, compounds of Formula A, or compounds of Formula B have improved bioavailability and can therefore be administered by oral administration.

In some embodiments, the formulations of the present invention are suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In some embodiments, the tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation of the invention include Tween® 80.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a compound according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the compounds disclosed herein are administered by inhalation. In some embodiments, formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the compounds used herein are formulated and dosed as dry powder. In some embodiments, the compounds used herein are formulated and dosed as a nebulized formulation. In some embodiments, the compounds used herein are formulated for delivery by a face mask. In some embodiments, the compounds used herein are formulated for delivery by a face tent.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

### V. Kits

Also provided herein are kits that includes a compound disclosed herein, a pharmaceutically acceptable salt, stereoisomer, mixture of stereoisomers or tautomer thereof. In some embodiments the kits described herein may comprise a label and/or instructions for use of the compound in the treatment of a disease or condition in a subject (e.g., human) in need thereof. In some embodiments, the disease or condition is viral infection.

In some embodiments, the kit may also comprise one or more additional therapeutic agents and/or instructions for use of additional therapeutic agents in combination with the compound of Formula I, compound of Formula A, or compound of Formula B in the treatment of the disease or condition in a subject (e.g., human) in need thereof.

In some embodiments, the kits provided herein comprises individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units may include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit may contain a single dosage unit and in others, multiple dosage units are present, such as the number of dosage units required for a specified regimen or period.

Also provided are articles of manufacture that include a compound of Formula I, compound of Formula A, or compound of Formula B, or a pharmaceutically acceptable salt, stereoisomer, mixture of stereoisomers or tautomer thereof, and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more compounds of the invention are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds disclosed herein are administered by inhalation or intravenously. In some embodiments, the compounds disclosed herein are administered orally. It will be appreciated that the preferred route may vary with for example the condition of the recipient.

In the methods of the present invention for the treatment of a viral infection, the compounds of the present invention can be administered at any time to a human who may come into contact with the virus or is already suffering from the viral infection. In some embodiments, the compounds of the present invention can be administered prophylactically to humans coming into contact with humans suffering from the viral infection or at risk of coming into contact with humans suffering from the viral infection, e.g., healthcare providers. In some embodiments, administration of the compounds of the present invention can be to humans testing positive for the viral infection but not yet showing symptoms of the viral infection. In some embodiments, administration of the compounds of the present invention can be to humans upon commencement of symptoms of the viral infection.

In some embodiments, the methods disclosed herein comprise event driven administration of the compound described herein, e.g., the compound of Formula I, compound of Formula A, or compound of Formula B, or a pharmaceutically acceptable salt thereof, to the subject.

As used herein, the terms “event driven” or “event driven administration” refer to administration of the compound described herein (e.g., the compound of Formula I, compound of Formula A, or compound of Formula B), or a pharmaceutically acceptable salt thereof, (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, the compound of Formula I, compound of Formula A, or compound of Formula B, or a pharmaceutically acceptable salt thereof, is administered before exposure of the subject to the virus.

In some embodiments, the compound of Formula I, compound of Formula A, or compound of Formula B, or a pharmaceutically acceptable salt thereof, is administered before and after exposure of the subject to the virus.

In some embodiments, the compound of Formula I, compound of Formula A, or compound of Formula B, or a pharmaceutically acceptable salt thereof, is administered after exposure of the subject to the virus.

An example of event driven dosing regimen includes administration of the compound of Formula I, compound of Formula A, or compound of Formula B, or a pharmaceutically acceptable salt thereof, within 24 to 2 hours prior to the virus, followed by administration of the compound of Formula I, compound of Formula A, or compound of Formula B, or a pharmaceutically acceptable salt, every 24 hours during the period of exposure, followed by a further administration of the compound of Formula I, compound of Formula A, or compound of Formula B, or a pharmaceutically acceptable salt thereof, after the last exposure, and one last administration of the compound of Formula I, compound of Formula A, or compound of Formula B, or a pharmaceutically acceptable salt thereof, 24 hours later.

A further example of an event driven dosing regimen includes administration of the compound of Formula I, compound of Formula A, or compound of Formula B, or a pharmaceutically acceptable salt thereof, within 24 hours before the viral exposure, then daily administration during the period of exposure, followed by a last administration approximately 24 hours later after the last exposure (which may be an increased dose, such as a double dose).

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of Formula I, compound of Formula A, or compound of Formula B, or a pharmaceutically acceptable salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per day, or from about 30 mg to about 300 mg per day, or from about 0.3 mg to about 30 mg per day, or from about 30 mg to about 300 mg per day.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula I, compound of Formula A, or compound of Formula B are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound provided herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein. In some embodiments, a compound disclosed herein is administered three times daily in a method disclosed herein.

In some embodiments, a compound disclosed herein is administered once daily in the total daily dose of 100-4000 mg/day. In some embodiments, a compound disclosed herein is administered twice daily in the total daily dose of 100-4000 mg/day. In some embodiments, a compound disclosed herein is administered three times daily in the total daily dose of 100-4000 mg/day.

The frequency of dosage of the compound of the present disclosure will be determined by the needs of the individual patient and can be, for example, once per day or twice, or more times, per day. Administration of the compound continues for as long as necessary to treat the viral infection. For example, a compound can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

The compounds of the present disclosure or the pharmaceutical compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

### VII. Methods of Use

The present disclosure also provides a method of treating or preventing a viral infection in a subject (e.g., human) in need thereof, the method comprising administering to the subject a compound described herein.

In some embodiments, the present disclosure provides a method of treating a viral infection in a subject (e.g., human) in need thereof, the method comprising administering to a subject in need thereof a compound described herein.

In some embodiments, the compound described herein is administered to the human via oral, intramuscular, intravenous, subcutaneous, or inhalation administration.

In some embodiments, the present disclosure provides for methods of treating or preventing a viral infection in a subject (e.g., human) in need thereof, the method comprising administering to the subject a compound disclosed herein and at least one additional active therapeutic or prophylactic agent.

In some embodiments, the present disclosure provides for methods of treating a viral infection in a subject (e.g., human) in need thereof, the method comprising administering to the subject a compound disclosed herein, and at least one additional active therapeutic or prophylactic agent.

In one embodiment, the present disclosure provides for methods of inhibiting a viral polymerase in a cell, the methods comprising contacting the cell infected a virus with a compound disclosed herein, whereby the viral polymerase is inhibited.

In one embodiment, the present disclosure provides for methods of inhibiting a viral polymerase in a cell, the methods comprising contacting the cell infected a virus with a compound disclosed herein, and at least one additional active therapeutic agent, whereby the viral polymerase is inhibited.

Also provided here are the uses of the compounds disclosed herein for use in treating or preventing a viral infection in a subject in need thereof. For example, provided herein are uses of the compounds disclosed herein for use in treating a viral infection in a subject in need thereof.

In some embodiments, the viral infection is a paramyxoviridae virus infection. As such, in some embodiments, the present disclosure provides methods for treating a paramyxoviridae infection in a subject (e.g., a human) in need thereof, the method comprising administering to the subject a compound disclosed herein. Paramyxoviridae viruses include, but are not limited to Nipah virus, Hendra virus, measles, mumps, and parainfluenze virus.

In some embodiments, the viral infection is a human parainfluenza virus, Nipah virus, Hendra virus, measles, or mumps infection.

In some embodiments, the viral infection is a pneumoviridae virus infection. As such, in some embodiments, the present disclosure provides a method of treating a pneumoviridae virus infection in a human in need thereof, the method comprising administering to the human a compound provided herein. Pneumoviridae viruses include, but are not limited to, respiratory snycytial virus and human metapneumovirus. In some embodiments, the pneumoviridae virus infection is a respiratory syncytial virus infection. In some embodiments, the pneumoviridae virus infection is human metapneumovirus infection.

In some embodiments, the present disclosure provides a compound disclosed herein, for use in the treatment of a pneumoviridae virus infection in a human in need thereof. In some embodiments, the pneumoviridae virus infection is a respiratory syncytial virus infection. In some embodiments, the pneumoviridae virus infection is human metapneumovirus infection.

In some embodiments, the present disclosure provides methods for treating a RSV infection in a human in need thereof, the method comprising administering to the human a compound provided herein. In some embodiments, the human is suffering from a chronic respiratory syncytial viral infection. In some embodiments, the human is acutely infected with RSV.

In some embodiments, a method of inhibiting RSV replication is provided, wherein the method comprises administering to a human in need thereof, a compound disclosed herein, wherein the administration is by inhalation.

In some embodiments, the present disclosure provides a method for reducing the viral load associated with RSV infection, wherein the method comprises administering to a human infected with RSV a compound disclosed herein.

In some embodiments, the viral infection is a picornaviridae virus infection. As such, in some embodiments, the present disclosure provides a method of treating a picornaviridae virus infection in a human in need thereof, the method comprising administering to the human a compound of the present disclosure. Picornaviridae viruses are eneteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection (HRV). In some embodiments, the Picornaviridae virus infection is HRV-A, HRV—B, or HRV—C infection.

In some embodiments, the viral infection is selected from the group consisting of Coxsackie A virus infection, Coxsackie A virus infection, enterovirus D68 infection, enterovirus B69 infection, enterovirus D70 infection, enterovirus A71 infection, and poliovirus infection.

In some embodiments, the present disclosure provides a compound, for use in the treatment of a picornaviridae virus infection in a human in need thereof. In some embodiments, the picornaviridae virus infection is human rhinovirus infection.

In some embodiments, the viral infection is a flaviviridae virus infection. As such, in some embodiments, the present disclosure provides a method of treating a flaviviridae virus infection in a human in need thereof, the method comprising administering to the human a compound described herein. Representative flaviviridae viruses include, but are not limited to, dengue, Yellow fever, West Nile, Zika, Japanese encephalitis virus, and Hepatitis C (HCV). In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a Japanese ensephalitis virus infection. In some embodiments, the flaviviridae virus infection is a hepatitis C virus infection.

In some embodiments, the flaviviridae virus infection is a dengue virus infection, yellow fever virus infection, West Nile virus infection, tick borne encephalitis, Kunjin Japanese encephalitis, St. Louis encephalitis, Murray valley encephalitis, Omsk hemorrhagic fever, bovine viral diarrhea, zika virus infection, or a HCV infection.

In some embodiments, the present disclosure provides use of a compound disclosed herein for treatment of a flaviviridae virus infection in a human in need thereof. In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a hepatitis C virus infection.

In some embodiments, the viral infection is a filoviridae virus infection. As such, in some embodiments, provided herein is a method of treating a filoviridae virus infection in a human in need thereof, the method comprising administering to the human a compound disclosed herein. Representative filoviridae viruses include, but are not limited to, ebola (variants Zaire, Bundibugio, Sudan, Tai forest, or Reston) and marburg. In some embodiments, the filoviridae virus infection is an ebola virus infection. In some embodiments, the filoviridae virus infection is a marburg virus infection.

In some embodiments, the present disclosure provides a compound for use in the treatment of a filoviridae virus infection in a human in need thereof. In some embodiments, the filoviridae virus infection is an ebola virus infection. In some embodiments, the filoviridae virus infection is a marburg virus infection.

In some embodiments, the viral infection is a coronavirus infection. As such, in some embodiments, provided herein is a method of treating a coronavirus infection in a human in need thereof, wherein the method comprises administering to the human a compound provided herein. In some embodiments, the coronavirus infection is a Severe Acute Respiratory Syndrome (SARS-CoV) infection, Middle Eastern Respiratory Syndrome (MERS) infection, SARS-CoV-2 infection, other human coronavirus (229E, NL63, OC43, HKU1, or WIV1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection. In some embodiments, the viral infection is a zoonotic coronavirus infection, In some embodiments, the viral infection is caused by a virus having at least 70% sequence homology to a viral polymerase selected from the group consisting of SARS-CoV polymerase, MERS-CoV polymerase and SARS-CoV-2. In some embodiments, the viral infection is caused by a virus having at least 80% sequence homology to a viral polymerase selected from the group consisting of SARS-CoV polymerase, MERS-CoV polymerase and SARS-CoV-2. In some embodiments, the viral infection is caused by a virus having at least 90% sequence homology to a viral polymerase selected from the group consisting of SARS-CoV polymerase, MERS-CoV polymerase and SARS-CoV-2. In some embodiments, the viral infection is caused by a virus having at least 95% sequence homology to a viral polymerase selected from the group consisting of SARS-CoV polymerase, MERS-CoV polymerase and SARS-CoV-2.

In some embodiments, the viral infection is caused by a variant of SARS-CoV-2, for example by the B.1.1.7 variant (the UK variant), B.1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-CoV-2. In some embodiments, the viral infection is caused by the B.1.351 variant of SARS-CoV-2. In some embodiments, the viral infection is caused by the P.1 variant of SARS-CoV-2.

In some embodiments, the present disclosure provides a compound for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-CoV-2 infection, other human coronavirus (229E, NL63, OC43, HKU1, or WIV1) infections, and zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection (COVID19).

In some embodiments, the viral infection is an arenaviridae virus infection. As such, in some embodiments, the disclosure provides a method of treating an arenaviridae virus infection in a human in need thereof, the method comprising administering to the human a compound disclosed herein. In some embodiments, the arenaviridae virus infection is a Lassa infection or a Junin infection.

In some embodiments, the present disclosure provides a compound for use in the treatment of an arenaviridae virus infection in a human in need thereof. In some embodiments, the arenaviridae virus infection is a Lassa infection or a Junin infection.

In some embodiments, the viral infection is an orthomyxovirus infection, for example, an influenza virus infection. In some embodiments, the viral infection is an influenza virus A, influenza virus B, or influenza virus C infection.

As described more fully herein, the compounds described herein can be administered with one or more additional therapeutic agent(s) to an individual (e.g., a human) infected with a viral infection. The additional therapeutic agent(s) can be administered to the infected individual at the same time as the compound of the present disclosure or before or after administration of the compound of the present disclosure.

### VIII. Combination Therapy

The compounds described herein can also be used in combination with one or more additional therapeutic agents. As such, also provided herein are methods of treatment of a viral infection in a subject in need thereof, wherein the methods comprise administering to the subject a compound disclosed therein and a therapeutically effective amount of one or more additional therapeutic or prophylactic agents.

In some embodiments, the additional therapeutic agent is an antiviral agent. Any suitable antiviral agent can be used in the methods described herein.

In some embodiments, the additional therapeutic agent a 2,5-Oligoadenylate synthetase stimulator, 5-HT 2a receptor antagonist, 5-Lipoxygenase inhibitor, ABL family tyrosine kinase inhibitor, Abl tyrosine kinase inhibitor, Acetaldehyde dehydrogenase inhibitor, Acetyl CoA carboxylase inhibitor, Actin antagonist, Actin modulator, Activity-dependent neuroprotector modulator, Adenosine A3 receptor agonist, Adrenergic receptor antagonist, Adrenomedullin ligand, Adrenomedullin ligand inhibitor, Advanced glycosylation product receptor antagonist, Advanced glycosylation product receptor modulator, AKT protein kinase inhibitor, Alanine proline rich secreted protein stimulator, Aldose reductase inhibitor, Alkaline phosphatase stimulator, Alpha 2 adrenoceptor antagonist, Alpha 2B adrenoceptor agonist, AMP activated protein kinase stimulator, AMPA receptor modulator, Amyloid protein deposition inhibitor, Androgen receptor antagonist, Angiotensin II AT-1 receptor antagonist, Angiotensin II AT-2 receptor agonist, Angiotensin II receptor modulator, Angiotensin converting enzyme 2 inhibitor, Angiotensin converting enzyme 2 modulator, Angiotensin converting enzyme 2 stimulator, Angiotensin receptor modulator, Annexin A5 stimulator, Anoctamin 1 inhibitor, Anti-coagulant, Anti-histamine, Anti-hypoxic, Anti-thrombotic, AP1 transcription factor modulator, Apelin receptor agonist, APOA1 gene stimulator, Apolipoprotein A1 agonist, Apolipoprotein B antagonist, Apolipoprotein B modulator, Apolipoprotein C3 antagonist, Aryl hydrocarbon receptor agonist, Aryl hydrocarbon receptor antagonist, ATP binding cassette transporter B5 modulator, Axl tyrosine kinase receptor inhibitor, Bactericidal permeability protein inhibitor, Basigin inhibitor, Basigin modulator, BCL2 gene inhibitor, BCL2L11 gene stimulator, Bcr protein inhibitor, Beta 1 adrenoceptor modulator, Beta 2 adrenoceptor agonist, Beta adrenoceptor agonist, Beta-arrestin stimulator, Blood clotting modulator, BMP10 gene inhibitor, BMP15 gene inhibitor, Bone morphogenetic protein-10 ligand inhibitor, Bone morphogenetic protein-15 ligand inhibitor, Bradykinin B2 receptor antagonist, Brain derived neurotrophic factor ligand, Bromodomain containing protein 2 inhibitor, Bromodomain containing protein 4 inhibitor, Btk tyrosine kinase inhibitor, C-reactive protein modulator, Ca2+ release activated Ca2+ channel 1 inhibitor, Cadherin-5 modulator, Calcium activated chloride channel inhibitor, Calcium channel modulator, Calpain-I inhibitor, Calpain-II inhibitor, Calpain-IX inhibitor, Cannabinoid CB2 receptor agonist, Cannabinoid receptor modulator, Casein kinase II inhibitor, CASP8-FADD-like regulator inhibitor, Caspase inhibitor, Catalase stimulator, CCL26 gene inhibitor, CCR2 chemokine antagonist, CCR5 chemokine antagonist, CD11a agonist, CD122 agonist, CD3 antagonist, CD4 agonist, CD40 ligand, CD40 ligand modulator, CD40 ligand receptor agonist, CD40 ligand receptor modulator, CD49d agonist, CD70 antigen modulator, CD73 agonist, CD73 antagonist, CD95 antagonist, CFTR inhibitor, CGRP receptor antagonist, Chemokine receptor-like 1 agonist, Chloride channel inhibitor, Chloride channel modulator, Cholera enterotoxin subunit B inhibitor, Cholesterol ester transfer protein inhibitor, Collagen modulator, Complement Cis subcomponent inhibitor, Complement C3 inhibitor, Complement C5 factor inhibitor, Complement C5a factor inhibitor, Complement Factor H stimulator, Complement cascade inhibitor, Complement factor C2 inhibitor, Complement factor D inhibitor, Connective tissue growth factor ligand inhibitor, Coronavirus nucleoprotein modulator, Coronavirus small envelope protein modulator, Coronavirus spike glycoprotein inhibitor, Coronavirus spike glycoprotein modulator, COVID19 envelope small membrane protein modulator, COVID19 non structural protein 8 modulator, COVID19 nucleoprotein modulator, COVID19 Protein 3a inhibitor, COVID19 replicase polyprotein 1a inhibitor, COVID19 replicase polyprotein 1a modulator, COVID19 replicase polyprotein 1ab inhibitor, COVID19 replicase polyprotein 1ab modulator, COVID19 Spike glycoprotein inhibitor, COVID19 Spike glycoprotein modulator, COVID19 structural glycoprotein modulator, CRF-2 receptor agonist, CSF-1 agonist, CSF-1 antagonist, CX3CR1 chemokine antagonist, CXC10 chemokine ligand inhibitor, CXC5 chemokine ligand inhibitor, CXCL1 gene modulator, CXCL2 gene modulator, CXCL3 gene modulator, CXCR1 chemokine antagonist, CXCR2 chemokine antagonist, CXCR4 chemokine antagonist, Cyclin D1 inhibitor, Cyclin E inhibitor, Cyclin-dependent kinase-1 inhibitor, Cyclin-dependent kinase-2 inhibitor, Cyclin-dependent kinase-5 inhibitor, Cyclin-dependent kinase-7 inhibitor, Cyclin-dependent kinase-9 inhibitor, Cyclooxygenase 2 inhibitor, Cyclooxygenase inhibitor, Cyclophilin inhibitor, Cysteine protease inhibitor, Cytochrome P450 3A4 inhibitor, Cytokine receptor antagonist, Cytotoxic T lymphocyte protein gene modulator, Cytotoxic T-lymphocyte protein-4 inhibitor, Cytotoxic T-lymphocyte protein-4 stimulator, DDX3 inhibitor, Dehydrogenase inhibitor, Dehydropeptidase-1 modulator, Deoxyribonuclease I stimulator, Deoxyribonuclease gamma stimulator, Deoxyribonuclease stimulator, Dihydroceramide delta 4 desaturase inhibitor, Dihydroorotate dehydrogenase inhibitor, Dipeptidyl peptidase I inhibitor, Dipeptidyl peptidase III inhibitor, Diuretic, DNA binding protein inhibitor, DNA methyltransferase inhibitor, Dopamine transporter inhibitor, E selectin antagonist, Ecto NOX disulfide thiol exchanger 2 inhibitor, EGFR gene inhibitor, Elongation factor 1 alpha 2 modulator, Endoplasmin modulator, Endoribonuclease DICER modulator, Endothelin ET-A receptor antagonist, Epidermal growth factor receptor antagonist, E-selectin antagonist, Estrogen receptor beta agonist, Estrogen receptor modulator, Eukaryotic initiation factor 4A-I inhibitor, Exo-alpha sialidase modulator, Exportin 1 inhibitor, Factor Ia modulator, Factor IIa modulator, Factor VII antagonist, Factor Xa antagonist, Factor XIa antagonist, FGF receptor antagonist, FGF-1 ligand, FGF-1 ligand inhibitor, FGF-2 ligand inhibitor, FGF1 receptor antagonist, FGF2 receptor antagonist, FGF3 receptor antagonist, Flt3 tyrosine kinase inhibitor, Fractalkine ligand inhibitor, Free fatty acid receptor 2 agonist, Free fatty acid receptor 3 agonist, furin inhibitors, Fyn tyrosine kinase inhibitor, FYVE finger phosphoinositide kinase inhibitor, G-protein coupled bile acid receptor 1 agonist, GABA A receptor modulator, Galectin-3 inhibitor, Gamma-secretase inhibitor, GDF agonist, Gelsolin stimulator, Glial cell neurotrophic factor ligand, Glucocorticoid receptor agonist, Glutathione peroxidase stimulator, GM-CSF ligand inhibitor, GM-CSF receptor agonist, GM-CSF receptor modulator, Griffithsin modulator, Growth regulated protein alpha ligand inhibitor, Grp78 calcium binding protein inhibitor, Heat shock protein HSP90 alpha inhibitor, Heat shock protein HSP90 beta inhibitor, Heat shock protein inhibitor, Heat shock protein stimulator, Hemagglutinin modulator, Hemoglobin modulator, Hemolysin alpha inhibitor, Heparanase inhibitor, Heparin agonist, Hepatitis B structural protein inhibitor, Hepatitis C virus NS5B polymerase inhibitor, HIF prolyl hydroxylase inhibitor, HIF prolyl hydroxylase-2 inhibitor, High mobility group protein B1 inhibitor, Histamine H1 receptor antagonist, Histamine H2 receptor antagonist, Histone deacetylase-6 inhibitor, Histone inhibitor, HIV protease inhibitor, HIV-1 gp120 protein inhibitor, HIV-1 protease inhibitor, HIV-1 reverse transcriptase inhibitor, HLA class I antigen modulator, HLA class II antigen modulator, Host cell factor modulator, Hsp 90 inhibitor, Human papillomavirus E6 protein modulator, Human papillomavirus E7 protein modulator, Hypoxia inducible factor inhibitor gene inhibitor, Hypoxia inducible factor-2 alpha modulator, I-kappa B kinase inhibitor, I-kappa B kinase modulator, ICAM-1 stimulator, IgG receptor FcRn large subunit p51 modulator, IL-12 receptor antagonist, IL-15 receptor agonist, IL-15 receptor modulator, IL-17 antagonist, IL-18 receptor accessory protein antagonist, IL-2 receptor agonist, IL-22 agonist, IL-23 antagonist, IL-6 receptor agonist, IL-6 receptor antagonist, IL-6 receptor modulator, IL-7 receptor agonist, IL-8 receptor antagonist, IL12 gene stimulator, IL8 gene modulator, Immunoglobulin G modulator, Immunoglobulin G1 agonist, Immunoglobulin G1 modulator, Immunoglobulin agonist, Immunoglobulin gamma Fc receptor I modulator, Immunoglobulin kappa modulator, Inosine monophosphate dehydrogenase inhibitor, Insulin sensitizer, Integrin agonist, Integrin alpha-4/beta-7 antagonist, Integrin alpha-V/beta-1 antagonist, Integrin alpha-V/beta-6 antagonist, Interferon agonist, Interferon alpha 14 ligand, Interferon alpha 2 ligand, Interferon alpha 2 ligand modulator, Interferon alpha ligand, Interferon alpha ligand inhibitor, Interferon alpha ligand modulator, Interferon beta ligand, Interferon gamma ligand inhibitor, Interferon gamma receptor agonist, Interferon gamma receptor antagonist, Interferon receptor modulator, Interferon type I receptor agonist, Interleukin 17A ligand inhibitor, Interleukin 17F ligand inhibitor, Interleukin 18 ligand inhibitor, Interleukin 22 ligand, Interleukin-1 beta ligand inhibitor, Interleukin-1 beta ligand modulator, Interleukin-1 ligand inhibitor, Interleukin-2 ligand, Interleukin-29 ligand, Interleukin-6 ligand inhibitor, Interleukin-7 ligand, Interleukin-8 ligand inhibitor, IRAK-4 protein kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Jun N terminal kinase modulator, Kallikrein modulator, Kelch like ECH associated protein 1 modulator, Kit tyrosine kinase inhibitor, KLKB1 gene inhibitor, Lactoferrin stimulator, Lanosterol-14 demethylase inhibitor, Lck tyrosine kinase inhibitor, Leukocyte Ig like receptor A4 modulator, Leukocyte elastase inhibitor, Leukotriene BLT receptor antagonist, Leukotriene D4 antagonist, Leukotriene receptor antagonist, Listeriolysin stimulator, Liver X receptor antagonist, Low molecular weight heparin, Lung surfactant associated protein B stimulator, Lung surfactant associated protein D modulator, Lyn tyrosine kinase inhibitor, Lyn tyrosine kinase stimulator, Lysine specific histone demethylase 1 inhibitor, Macrophage migration inhibitory factor inhibitor, Mannan-binding lectin serine protease inhibitor, Mannan-binding lectin serine protease-2 inhibitor, MAO B inhibitor, MAP kinase inhibitor, MAPK gene modulator, Matrix metalloprotease modulator, Maxi K potassium channel inhibitor, MCL1 gene inhibitor, MEK protein kinase inhibitor, MEK-1 protein kinase inhibitor, Melanocortin MC1 receptor agonist, Melanocortin MC3 receptor agonist, Metalloprotease-12 inhibitor, METTL3 gene inhibitor, Moesin inhibitor, Moesin modulator, Monocyte chemotactic protein 1 ligand inhibitor, Monocyte differentiation antigen CD14 inhibitor, mRNA cap guanine N7 methyltransferase modulator, mTOR complex 1 inhibitor, mTOR complex 2 inhibitor, mTOR inhibitor, Mucolipin modulator, Muscarinic receptor antagonist, Myeloperoxidase inhibitor, NACHT LRR PYD domain protein 3 inhibitor, NAD synthase modulator, NADPH oxidase inhibitor, Neuropilin 2 modulator, Neuroplastin inhibitor, NFE2L2 gene stimulator, NK cell receptor agonist, NK1 receptor antagonist, NMDA receptor antagonist, NMDA receptor epsilon 2 subunit inhibitor, Non receptor tyrosine kinase TYK2 antagonist, Non-nucleoside reverse transcriptase inhibitor, nsp12 polymerase inhibitor, Nuclear erythroid 2-related factor 2 stimulator, Nuclear factor kappa B inhibitor, Nuclear factor kappa B modulator, Nuclease stimulator, Nucleolin inhibitor, Nucleoprotein inhibitor, Nucleoprotein modulator, Nucleoside reverse transcriptase inhibitor, Opioid receptor agonist, Opioid receptor antagonist, Opioid receptor mu modulator, Opioid receptor sigma antagonist 1, ORFlab polyprotein inhibitor, Ornithine decarboxylase inhibitor, Outer membrane protein inhibitor, OX40 ligand, p38 MAP kinase alpha inhibitor, p38 MAP kinase inhibitor, p38 MAP kinase modulator, p53 tumor suppressor protein stimulator, Palmitoyl protein thioesterase 1 inhibitor, Papain inhibitor, PARP inhibitor, PARP modulator, PDE 10 inhibitor, PDE 3 inhibitor, PDE 4 inhibitor, PDGF receptor alpha antagonist, PDGF receptor antagonist, PDGF receptor beta antagonist, Peptidyl-prolyl cis-trans isomerase A inhibitor, Peroxiredoxin 6 modulator, PGD2 antagonist, PGI2 agonist, P-glycoprotein inhibitor, Phosphoinositide 3-kinase inhibitor, Phosphoinositide-3 kinase delta inhibitor, Phosphoinositide-3 kinase gamma inhibitor, Phospholipase A2 inhibitor, PIKfyve inhibitor, Plasma kallikrein inhibitor, Plasminogen activator inhibitor 1 inhibitor, Platelet inhibitor, Platelet glycoprotein VI inhibitor, Polo-like kinase 1 inhibitor, Poly ADP ribose polymerase 1 inhibitor, Poly ADP ribose polymerase 2 inhibitor, Polymerase cofactor VP35 inhibitor, PPAR alpha agonist, Progesterone receptor agonist, Programmed cell death protein 1 modulator, Prolyl hydroxylase inhibitor, Prostaglandin E synthase-1 inhibitor, Protease inhibitor, Proteasome inhibitor, Protein arginine deiminase IV inhibitor, Protein tyrosine kinase inhibitor, Protein tyrosine phosphatase beta inhibitor, Protein tyrosine phosphatase-2C inhibitor, Proto-oncogene Mas agonist, Purinoceptor antagonist, Raf protein kinase inhibitor, RANTES ligand, Ras gene inhibitor, Retinoate receptor responder protein 2 stimulator, Rev protein modulator, Ribonuclease stimulator, RIP-1 kinase inhibitor, RNA helicase inhibitor, RNA polymerase inhibitor, RNA polymerase modulator, S phase kinase associated protein 2 inhibitor, SARS coronavirus 3C protease like inhibitor, Serine protease inhibitor, Serine threonine protein kinase ATR inhibitor, Serine threonine protein kinase TBKi inhibitor, Serum amyloid A protein modulator, Signal transducer CD24 stimulator, Sirtuin inhibitor, Sodium channel stimulator, Sodium glucose transporter-2 inhibitor, Sphingosine kinase 1 inhibitor, Sphingosine kinase 2 inhibitor, Sphingosine kinase inhibitor, Sphingosine-i-phosphate receptor-1 agonist, Sphingosine-1-phosphate receptor-1 antagonist, Sphingosine-1-phosphate receptor-1 modulator, Sphingosine-1-phosphate receptor-5 agonist, Sphingosine-1-phosphate receptor-5 modulator, Spike glycoprotein inhibitor, Src tyrosine kinase inhibitor, STAT-1 modulator, STAT-3 inhibitor, STAT-5 inhibitor, STAT3 gene inhibitor, Stem cell antigen-1 inhibitor, Stimulator of interferon genes protein stimulator, Sulfatase inhibitor, Superoxide dismutase modulator, Superoxide dismutase stimulator, Syk tyrosine kinase inhibitor, T cell immunoreceptor Ig ITIM protein inhibitor, T cell receptor agonist, T cell surface glycoprotein CD28 inhibitor, T-cell differentiation antigen CD6 inhibitor, T-cell surface glycoprotein CD8 stimulator, T-cell transcription factor NFAT modulator, Tankyrase-1 inhibitor, Tankyrase-2 inhibitor, Tek tyrosine kinase receptor stimulator, Telomerase modulator, Tetanus toxin modulator, TGF beta receptor antagonist, TGFB2 gene inhibitor, Thymosin beta 4 ligand, Thyroid hormone receptor beta agonist, Tissue factor inhibitor, Tissue plasminogen activator modulator, Tissue plasminogen activator stimulator, TLR agonist, TLR modulator, TLR-2 agonist, TLR-2 antagonist, TLR-3 agonist, TLR-4 agonist, TLR-4 antagonist, TLR-6 agonist, TLR-7 agonist, TLR-7 antagonist, TLR-8 antagonist, TLR-9 agonist, TMPRSS2 gene inhibitor, TNF alpha ligand inhibitor, TNF alpha ligand modulator, TNF binding agent, TNF gene inhibitor, Topoisomerase inhibitor, Transcription factor EB stimulator, Transferrin modulator, Transketolase inhibitor, Translocation associated protein inhibitor, Transmembrane serine protease 2 inhibitor, Transthyretin modulator, TREM receptor 1 antagonist, TRP cation channel C1 modulator, TRP cation channel C6 inhibitor, TRP cation channel V6 inhibitor, Trypsin 1 inhibitor, Trypsin 2 inhibitor, Trypsin 3 inhibitor, Trypsin inhibitor, Tubulin alpha inhibitor, Tubulin beta inhibitor, Tumor necrosis factor 14 ligand inhibitor, TYK2 gene inhibitor, Type I IL-1 receptor antagonist, Tyrosine protein kinase ABL1 inhibitor, Ubiquinol cytochrome C reductase 14 kDa inhibitor, Ubiquitin ligase modulator, Unspecified GPCR agonist, Unspecified cytokine receptor modulator, Unspecified enzyme stimulator, Unspecified gene inhibitor, Unspecified receptor modulator, Urokinase plasminogen activator inhibitor, Vascular cell adhesion protein 1 agonist, Vasodilator, VEGF ligand inhibitor, VEGF receptor antagonist, VEGF-1 receptor antagonist, VEGF-1 receptor modulator, VEGF-2 receptor antagonist, VEGF-3 receptor antagonist, Vimentin inhibitor, Vimentin modulator, VIP receptor agonist, Viral envelope protein inhibitor, Viral protease inhibitor, Viral protease modulator, Viral protein target modulator, Viral ribonuclease inhibitor, Viral structural protein modulator, Vitamin D3 receptor agonist, X-linked inhibitor of apoptosis protein inhibitor, Xanthine oxidase inhibitor, or Zonulin inhibitor.

In some embodiments, the compounds and compositions of the present disclosure may be administered in combination with a Sars-Cov-2 treatment, such as parenteral fluids (including dextrose saline and Ringer's lactate), nutrition, antibiotics (including azithromycin, metronidazole, amphotericin B, amoxicillin/clavulanate, trimethoprim/sulfamethoxazole, R-327 and cephalosporin antibiotics, such as ceftriaxone and cefuroxime), antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as dexamethasone, methylprednisolone, prednisone, mometasone, immunomodulatory medications (eg interferon), vaccines, and pain medications.

In some embodiments, the additional therapeutic agent is an Abl tyrosine kinase inhibitor, such as radotinib or imatinib.

In some embodiments, the additional therapeutic agent is an acetaldehyde dehydrogenase inhibitor, such as ADX-629.

In some embodiments, the additional therapeutic agent is an adenosine A3 receptor agonist, such as piclidenoson.

In some embodiments, the additional therapeutic agent is an adrenomedullin ligand such as adrenomedullin.

In some embodiments, the additional therapeutic agent is a p38 MAPK+PPAR gamma agonist/insulin sensitizer such as KIN-001.

In some embodiments, the additional therapeutic agent is a PPAR alpha agonist such as DWTC-5101 (fenofibrate choline).

In some embodiments, the additional therapeutic agent is a cyclophilin inhibitor such as rencofilstat.

In some embodiments, the additional therapeutic is a p38 MAP kinase inhibitor such as PRX-201 or Gen-1124.

In some embodiments, the additional therapeutic agent is an aldose reductase inhibitor, such as caficrestat.

In some embodiments, the additional therapeutic agent is an AMPA receptor modulator, such as traneurocin.

In some embodiments, the additional therapeutic agent is an annexin A5 stimulator, such as AP-01 or SY-005.

In some embodiments, the additional therapeutic agent is an apelin receptor agonist, such as CB-5064MM.

In some embodiments, the additional therapeutic agent is an anti-coagulant, such as heparins (heparin and low molecular weight heparin), aspirin, apixaban, dabigatran, edoxaban, argatroban, enoxaparin, or fondaparinux.

In some embodiments, the additional therapeutic agent is an androgen receptor antagonist such as bicalutamide, deutenzalutamide, enzalutamide, or pruxelutamide (proxalutamide).

In some embodiments, the additional therapeutic agent is anti-hypoxic, such as trans-sodium crocetinate.

In some embodiments, the additional therapeutic agent is an anti-thrombotic, such as defibrotide, rivaroxaban, alteplase, tirofiban, clopidogrel, prasugrel, bemiparin, bivalirudin, sulodexide, or tenecteplase.

In some embodiments, the additional therapeutic agent is an antihistamine, such as cloroperastine or clemastine.

In some embodiments, the additional therapeutic agent is an apolipoprotein A1 agonist, such as CER-001.

In some embodiments, the additional therapeutic agent is a phospholipase A2 inhibitor, such as icosapent ethyl.

In some embodiments, the additional therapeutic agent is an axl tyrosine kinase receptor inhibitor, such as bemcentinib.

In some embodiments, the additional therapeutic agent is a corticosteroid/beta 2 adrenoceptor agonist, such as budesonide+formoterol fumarate.

In some embodiments, the additional therapeutic agent is a BET bromodomain inhibitor/APOA1 gene stimulator such as apabetalone.

In some embodiments, the additional therapeutic agent is a blood clotting modulator, such as lanadelumab.

In some embodiments, the additional therapeutic agent is a bradykinin B2 receptor antagonist, such as icatibant.

In some embodiments, the additional therapeutic agent is an EGFR gene inhibitor/Btk tyrosine kinase inhibitor, such as abivertinib.

In some embodiments, the additional therapeutic agent is a Btk tyrosine kinase inhibitor, such as ibrutinib or zanubrutinib.

In some embodiments, the additional therapeutic agent is a calpain-III/IX inhibitor, such as BLD-2660.

In some embodiments, the additional therapeutic agent is a cannabinoid CB2 receptor agonist, such as onternabez or PPP-003.

In some embodiments, the additional therapeutic agent is a Ca2+ release activated Ca2+ channel 1 inhibitor, such as zegocractin (CM-4620).

In some embodiments, the additional therapeutic agent is an ATR inhibitor, such as berzosertib.

In some embodiments, the additional therapeutic agent is a cadherin-5 modulator, such as FX-06.

In some embodiments, the additional therapeutic agent is a casein kinase II inhibitor, such as silmitasertib.

In some embodiments, the additional therapeutic agent is a caspase inhibitor, such as emricasan.

In some embodiments, the additional therapeutic agent is a catalase stimulator/superoxide dismutase stimulator, such as MP-1032.

In some embodiments, the additional therapeutic agent is a CCR2 chemokine antagonist/CCR5 chemokine antagonist such as cenicriviroc.

In some embodiments, the additional therapeutic agent is a CCR5 chemokine antagonist, such as maraviroc or leronlimab.

In some embodiments, the additional therapeutic agent is a CD122 agonist/IL-2 receptor agonist, such as bempegaldesleukin.

In some embodiments, the additional therapeutic agent is a CD73 agonist/interferon beta ligand, such as FP-1201.

In some embodiments, the additional therapeutic agent is a cholesterol ester transfer protein inhibitor, such as dalcetrapib.

In some embodiments, the additional therapeutic agent is a Mannan-binding lectin serine protease/complement Cls subcomponent inhibitor/myeloperoxidase inhibitor, such as RLS-0071.

In some embodiments, the additional therapeutic agent is a complement C5 factor inhibitor/leukotriene BLT receptor antagonist, such as nomacopan.

In some embodiments, the additional therapeutic agent is a complement C5 factor inhibitor, such as eculizumab, STSA-1002, zilucoplan.

In some embodiments, the additional therapeutic agent is a CXCR4 chemokine antagonist, such as plerixafor or motixafortide.

In some embodiments, the additional therapeutic agent is a cytochrome P450 3A4 inhibitor/peptidyl-prolyl cis-trans isomerase A inhibitor, such as alisporivir.

In some embodiments, the additional therapeutic agent is a cysteine protease inhibitor, such as SLV-213.

In some embodiments, the additional therapeutic agent is a dihydroorotate dehydrogenase inhibitor, such as Meds-433, brequinar, RP-7214, farudostat or emvododstat.

In some embodiments, the additional therapeutic agent is a dehydropeptidase-1 modulator, such as Metablok.

In some embodiments, the additional therapeutic agent is a dihydroorotate dehydrogenase inhibitor/IL-17 antagonist, such as vidofludimus.

In some embodiments, the additional therapeutic agent is a diuretic, such as an aldosterone antagonist, such as spironolactone.

In some embodiments, the additional therapeutic agent is a deoxyribonuclease I stimulator, such as GNR-039 or dornase alfa.

In some embodiments, the additional therapeutic agent is a NET inhibitor, such as NTR-441.

In some embodiments, the additional therapeutic agent is a dihydroceramide delta 4 desaturase inhibitor/sphingosine kinase 2 inhibitor, such as opaganib.

In some embodiments, the additional therapeutic agent is a DNA methyltransferase inhibitor, such as azacytidine.

In some embodiments, the additional therapeutic agent is an LXR antagonist, such as larsucosterol.

In some embodiments, the additional therapeutic agent is a dipeptidyl peptidase I inhibitor, such as brensocatib.

In some embodiments, the additional therapeutic agent is a protein arginine deiminase IV inhibitor, such as JBI-1044.

In some embodiments, the additional therapeutic agent is an elongation factor 1 alpha 2 modulator, such as plitidepsin.

In some embodiments, the additional therapeutic agent is a eukaryotic initiation factor 4A-I inhibitor, such as zotatifin.

In some embodiments, the additional therapeutic agent is an exo-alpha sialidase modulator, such as DAS-181.

In some embodiments, the additional therapeutic agent is an exportin 1 inhibitor, such as selinexor.

In some embodiments, the additional therapeutic agent is a fractalkine ligand inhibitor, such as KAND-567.

In some embodiments, the additional therapeutic agent is a FYVE finger phosphoinositide kinase inhibitor/IL-12 receptor antagonist/IL-23 antagonist, such as apilimod dimesylate.

In some embodiments, the additional therapeutic agent is a GABA A receptor modulator, such as brexanolone.

In some embodiments, the additional therapeutic agent is a glucocorticoid receptor agonist, such as ciclesonide, hydrocortisone, dexamethasone, dexamethasone phosphate, or 101-PGC-005.

In some embodiments, the additional therapeutic agent is a GM-CSF receptor agonist, such as sargramostim.

In some embodiments, the additional therapeutic agent is a GPCR agonist, such as esuberaprost sodium.

In some embodiments, the additional therapeutic agent is a Griffithsin modulator, such as Q-Griffithsin.

In some embodiments, the additional therapeutic agent is a leukotriene D4 antagonist, such as montelukast.

In some embodiments, the additional therapeutic agent is a histamine H1 receptor antagonist, such as ebastine, tranilast, levocetirizine dihydrochloride.

In some embodiments, the additional therapeutic agent is a histamine H2 receptor antagonist, such as famotidine.

In some embodiments, the additional therapeutic agent is a heat shock protein stimulator/insulin sensitizer/PARP inhibitor, such as BGP-15.

In some embodiments, the additional therapeutic agent is a histone inhibitor, such as STC-3141.

In some embodiments, the additional therapeutic agent is a histone deacetylase-6 inhibitor, such as CKD-506.

In some embodiments, the additional therapeutic agent is a HIF prolyl hydroxylase-2 inhibitor, such as desidustat.

In some embodiments, the additional therapeutic agent is an HIF prolyl hydroxylase inhibitor, such as vadadustat.

In some embodiments, the additional therapeutic agent is an IL-8 receptor antagonist, such as reparixin.

In some embodiments, the additional therapeutic agent is an IL-7 receptor agonist, such as CYT-107.

In some embodiments, the additional therapeutic agent is an IL-7 receptor agonist/interleukin-7 ligand, such as efineptakin alfa.

In some embodiments, the additional therapeutic agent is an IL-22 agonist, such as efmarodocokin alfa.

In some embodiments, the additional therapeutic agent is an IL-22 agonist/interleukin 22 ligand, such as F-652.

In some embodiments, the additional therapeutic agent is targeted to IL-33, such as tozorakimab.

In some embodiments, the additional therapeutic is an IL-15 agonist such as nogapendekin alfa.

In some embodiments, the additional therapeutic agent is an integrin alpha-V/beta-1 antagonist/integrin alpha-V/beta-6 antagonist, such as bexotegrast.

In some embodiments, the additional therapeutic agent is an interferon alpha 2 ligand, such as interferon alfa-2b or Virafin.

In some embodiments, the additional therapeutic agent is an interferon beta ligand, such as interferon beta-la follow-on biologic, interferon beta-1b, or SNG-001.

In some embodiments, the additional therapeutic agent is an interferon receptor modulator, such as peginterferon lambda-la.

In some embodiments, the additional therapeutic agent is an interleukin-2 ligand, such as aldesleukin.

In some embodiments, the additional therapeutic agent is an IRAK-4 protein kinase inhibitor, such as zimlovisertib.

In some embodiments, the additional therapeutic agent is a JAK inhibitor, for example the additional therapeutic agent is baricitinib, filgotinib, jaktinib, tofacitinib, or nezulcitinib (TD-0903).

In some embodiments, the additional therapeutic agent is a neutrophil elastase inhibitor, such as alvelestat.

In some embodiments, the additional therapeutic agent is a lung surfactant associated protein D modulator, such as AT-100.

In some embodiments, the additional therapeutic agent is a plasma kallikrein inhibitor, such as donidalorsen.

In some embodiments, the additional therapeutic agent is a lysine specific histone demethylase 1/MAO B inhibitor, such as vafidemstat.

In some embodiments, the additional therapeutic agent is a Mannan-binding lectin serine protease inhibitor, such as conestat alfa.

In some embodiments, the additional therapeutic agent is a maxi K potassium channel inhibitor, such as ENA-001.

In some embodiments, the additional therapeutic agent is a MEK protein kinase inhibitor, such as zapnometinib.

In some embodiments, the additional therapeutic agent is a MEK-1 protein kinase inhibitor/Ras gene inhibitor, such as antroquinonol.

In some embodiments, the additional therapeutic agent is a melanocortin MC1 receptor agonist, such as PL-8177

In some embodiments, the additional therapeutic agent is a melanocortin MC1/MC3 receptor agonist, such as resomelagon acetate.

In some embodiments, the additional therapeutic agent is a matrix metalloprotease-12 inhibitor, such as FP-025.

In some embodiments, the additional therapeutic agent is a NACHT LRR PYD domain protein 3 inhibitor, such as dapansutrile, DFV-890, or ZYIL-1.

In some embodiments, the additional therapeutic agent is a NADPH oxidase inhibitor, such as isuzinaxib.

In some embodiments, the additional therapeutic agent is a neuropilin 2 modulator, such as efzofitimod.

In some embodiments, the additional therapeutic agent is an NK1 receptor antagonist, such as aprepitant or tradipitant.

In some embodiments, the additional therapeutic agent is an NMDA receptor antagonist, such as transcrocetin or ifenprodil.

In some embodiments, the additional therapeutic agent is a nuclear factor kappa B inhibitor/p38 MAP kinase inhibitor, such as zenuzolac.

In some embodiments, the additional therapeutic agent is an ornithine decarboxylase inhibitor, such as eflornithine.

In some embodiments, the additional therapeutic agent is an opioid receptor sigma antagonist 1, such as MR-309.

In some embodiments, the additional therapeutic agent is a PGD2 antagonist, such as asapiprant.

In some embodiments, the additional therapeutic agent is a PDGF receptor antagonist/TGF beta receptor antagonist/p38 MAP kinase inhibitor, such as deupirfenidone.

In some embodiments, the additional therapeutic agent is a phospholipase A2 inhibitor, such as varespladib methyl.

In some embodiments, the additional therapeutic agent is a phosphoinositide 3-kinase inhibitor/mTOR complex inhibitor, such as dactolisib.

In some embodiments, the additional therapeutic agent is a mTOR inhibitor, such as sirolimus.

In some embodiments, the additional therapeutic agent is a phosphoinositide-3 kinase delta/gamma inhibitor, such as duvelisib.

In some embodiments, the additional therapeutic agent is a PIKfyve inhibitor, such as VRG-101.

In some embodiments, the additional therapeutic agent is a plasminogen activator inhibitor 1 inhibitor, such as TM-5614.

In some embodiments, the additional therapeutic agent is a protein tyrosine phosphatase beta inhibitor, such as razuprotafib.

In some embodiments, the additional therapeutic agent is a RIP-1 kinase inhibitor, such as eclitasertib (DNL-758) or SIR-0365.

In some embodiments, the additional therapeutic agent is a Rev protein modulator, such as obefazimod.

In some embodiments, the additional therapeutic agent is an S phase kinase associated protein 2 inhibitor, such as niclosamide, CP—COV3, SCAI-502 or DWRX-2003.

In some embodiments, the additional therapeutic agent is a signal transducer CD24 stimulator, such as EXO-CD24.

In some embodiments, the additional therapeutic agent is a sodium glucose transporter-2 inhibitor, such as dapagliflozin propanediol.

In some embodiments, the additional therapeutic agent is a sodium channel stimulator, such as solnatide.

In some embodiments, the additional therapeutic agent is a sphingosine-1-phosphate receptor-1 agonist/sphingosine-1-phosphate receptor-5 agonist, such as ozanimod.

In some embodiments, the additional therapeutic agent is a non-steroidal anti-inflammatory drug, such as Ampion.

In some embodiments, the additional therapeutic agent is a superoxide dismutase stimulator, such as avasopasem manganese.

In some embodiments, the additional therapeutic agent is a Syk tyrosine kinase inhibitor, such as fostamatinib disodium.

In some embodiments, the additional therapeutic agent is a Tie2 tyrosine kinase receptor agonist, such as AV-001.

In some embodiments, the additional therapeutic agent is a TGFB2 gene inhibitor, such as trabedersen.

In some embodiments, the additional therapeutic agent is a tissue factor inhibitor, such as AB-201.

In some embodiments, the additional therapeutic agent is a TLR-3 agonist, such as rintatolimod.

In some embodiments, the additional therapeutic agent is a TLR-4 antagonist, such as ApTLR-4FT, EB-05, or eritoran.

In some embodiments, the additional therapeutic agent is a TLR-7/8 antagonist, such as enpatoran.

In some embodiments, the additional therapeutic agent is a TLR-2/6 agonist, such as INNA-051.

In some embodiments, the additional therapeutic agent is a TLR-7 agonist, such as PRTX-007 or APR-002.

In some embodiments, the additional therapeutic agent is a TLR agonist, such as PUL-042.

In some embodiments, the additional therapeutic agent is a TLR-4 agonist, such as REVTx-99.

In some embodiments, the additional therapeutic agent is a TLR-2/4 antagonist, such as VB-201.

In some embodiments, the additional therapeutic agent is a TNF alpha ligand inhibitor, such as pegipanermin.

In some embodiments, the additional therapeutic agent is a type I IL-1 receptor antagonist, such as anakinra.

In some embodiments, the additional therapeutic agent is a TREM receptor 1 antagonist, such as nangibotide.

In some embodiments, the additional therapeutic agent is a trypsin inhibitor, such as ulinastatin.

In some embodiments, the additional therapeutic agent is a tubulin inhibitor such as sabizabulin, CCI-001, PCNT-13, CR-42-24, albendazole, entasobulin, SAR-132885, or ON-24160.

In some embodiments, the additional therapeutic agent is a VIP receptor agonist, such as aviptadil.

In some embodiments, the additional therapeutic agent is a xanthine oxidase inhibitor, such as oxypurinol.

In some embodiments, the additional therapeutic agent is a vasodilator, such as iloprost, epoprostenol (VentaProst), zavegepant, TXA-127, USB-002, ambrisentan, nitric oxide nasal spray (NORS), pentoxifylline, propranolol, RESP301, sodium nitrite, or dipyridamole.

In some embodiments, the additional therapeutic agent is a vitamin D3 receptor agonist, such as cholecalciferol.

In some embodiments, the additional therapeutic agent is a zonulin inhibitor, such as larazotide acetate.

In some embodiments, the additional therapeutic agent is a synthetic retinoid derivative, such as fenretinide.

In some embodiments, the additional therapeutic agent is a glucose metabolism inhibitor such as WP-1122 or WP-1096.

In some embodiments, the additional therapeutic agent is adalimumab, AT-H201, 2-deoxy-D-glucose, AD-17002, AIC-649, AMTX-100, astodrimer, AZD-1656, belapectin, bitespiramycin, bucillamine, budesonide, CNM-AgZn-17, Codivir, CT-38, danicopan, didodecyl methotrexate, DW-2008S (DW-2008), EDP-1815, EG-009A, Fabencov, Gamunex, genistein, GLS-1200, hzVSF-v13, imidazolyl ethanamide pentandioic acid, IMM-101, MAS-825, MRG-001, Nasitrol, Nylexa, olverembatinib, OP-101, OPN-019, Orynotide rhesus theta defensin-1, pyronaridine+artesunate, dapsone, RPH-104, sodium pyruvate, Sulforadex, tafenoquine, TB-006, telacebec, Tempol, TL-895, thimesoral, trimodulin, XC-221, XC-7, zunsemetinib, metformin glycinate, lucinactant, EOM-613, mosedipimod, ivermectin, leflunomide, ibudilast, RBT-9, raloxifene, prothione, gemcabene, or idronoxil.

In some embodiments, the additional therapeutic agent is a CD73 antagonist, such as AK-119.

In some embodiments, the additional therapeutic agent is a CD95 protein fusion, such as asunercept.

In some embodiments, the additional therapeutic agent is a complement factor C2 modulator, such as ARGX-117.

In some embodiments, the additional therapeutic agent is a complement C3 inhibitor, such as AMY-101 or NGM-621.

In some embodiments, the additional therapeutic agent is a CXC10 chemokine ligand inhibitor, such as EB-06.

In some embodiments, the additional therapeutic agent is a cytotoxic T-lymphocyte protein-4 fusion protein, such as abatacept.

In some embodiments, the additional therapeutic agent is an anti-S. Aureus antibody, such as tosatoxumab.

In some embodiments, the additional therapeutic agent is an anti-LPS antibody, such as IMM-124-E.

In some embodiments, the additional therapeutic agent is an adrenomedullin ligand inhibitor, such as enibarcimab.

In some embodiments, the additional therapeutic agent is a basigin inhibitor, such as meplazumab.

In some embodiments, the additional therapeutic agent is a CD3 antagonist, such as foralumab.

In some embodiments, the additional therapeutic agent is a connective tissue growth factor ligand inhibitor, such as PRS-220, pamrevlumab.

In some embodiments, the additional therapeutic agent is a complement C5a factor inhibitor, such as BDB-1 or vilobelimab.

In some embodiments, the additional therapeutic agent is a complement C5 factor inhibitor, such as ravulizumab.

In some embodiments, the additional therapeutic agent is a mannan-binding lectin serine protease-2 inhibitor, such as narsoplimab.

In some embodiments, the additional therapeutic agent is a GM-CSF modulator, such as STSA-1005, gimsilumab, namilumab, plonmarlimab, otilimab, or lenzilumab.

In some embodiments, the additional therapeutic agent is a heat shock protein inhibitor/IL-6 receptor antagonist, such as siltuximab.

In some embodiments, the additional therapeutic agent is an IL-6 receptor antagonist, such as clazakizumab, levilimab, olokizumab, tocilizumab, or sirukumab.

In some embodiments, the additional therapeutic agent is an IL-8 receptor antagonist, such as BMS-986253.

In some embodiments, the additional therapeutic agent is an interleukin-1 beta ligand inhibitor, such as canakinumab.

In some embodiments, the additional therapeutic agent is an interferon gamma ligand inhibitor, such as emapalumab.

In some embodiments, the additional therapeutic agent is an anti-ILT7 antibody, such as daxdilimab.

In some embodiments, the additional therapeutic agent is a monocyte differentiation antigen CD14 inhibitor, such as atibuclimab.

In some embodiments, the additional therapeutic agent is a plasma kallikrein inhibitor, such as lanadelumab.

In some embodiments, the additional therapeutic agent is a platelet glycoprotein VI inhibitor, such as glenzocimab.

In some embodiments, the additional therapeutic agent is a T-cell differentiation antigen CD6 inhibitor, such as itolizumab.

In some embodiments, the additional therapeutic agent is a TNF alpha ligand inhibitor/TNF binding agent, such as infliximab.

In some embodiments, the additional therapeutic agent is an anti-LIGHT antibody, such as AVTX-002.

In some embodiments, the additional therapeutic agent is IMC-2 (valacyclovir+celecoxib), or AXA-1125.

In some embodiments, the additional therapeutic agent is COVID-HIG.

In some embodiments, a compound of the disclosure, or a pharmaceutically acceptable salt thereof, is co-administered with one or more agents useful for the treatment and/or prophylaxis of COVID-19.

Non-limiting examples of such agents include corticosteroids, such as dexamethasone, hydrocortisone, methylprednisolone, or prednisone; interleukin-6 (IL-6) receptor blockers, such as tocilizumab or sarilumab; Janus kinase (JAK) inhibitors, such as baricitinib, ruxolitinib, or tofacitinib; and antiviral agents, such as molnupiravir, sotrovimab, or remdesivir.

In further embodiments, a compound of the disclosure, or a pharmaceutically acceptable salt thereof, is co-administered with two or more agents useful for the treatment of COVID-19. Agents useful for the treatment and/or prophylaxis of COVID-19 include but are not limited to a compound of the disclosure and two additional therapeutic agents, such as nirmatrelvir and ritonavir, casirivimab and imdevimab, or ruxolitinib and tofacitinib.

In some embodiments, the additional therapeutic agent is an antiviral agent. In some embodiments, the antiviral agent is an entry inhibitor. In some embodiments, the antiviral agent is a protease inhibitor. In some embodiments, the antiviral agent is an RNA polymerase inhibitor. In some embodiments, the additional therapeutic agent is an RNA-dependent RNA polymerase (RdRp) inhibitor.

In some embodiments, the antiviral agent is selected from angiotensin converting enzyme 2 inhibitors, angiotensin converting enzyme 2 modulators, angiotensin converting enzyme 2 stimulators, angiotensin II AT-2 receptor agonists, angiotensin II AT-2 receptor antagonists, angiotensin II receptor modulators, coronavirus nucleoprotein modulators, coronavirus small envelope protein modulators, coronavirus spike glycoprotein inhibitors, coronavirus spike glycoprotein modulators, SARS-CoV-2 envelope small membrane protein inhibitors, SARS-CoV-2 envelope small membrane protein modulators, SARS-CoV-2 MPro inhibitors, SARS-CoV-2 non structural protein 8 modulators, SARS-CoV-2 nucleoprotein inhibitors, SARS-CoV-2 nucleoprotein modulators, SARS-CoV-2 protein 3a inhibitors, SARS-CoV-2 replicase polyprotein 1a inhibitors, SARS-CoV-2 replicase polyprotein 1a modulators, SARS-CoV-2 replicase polyprotein 1ab inhibitors, SARS-CoV-2 replicase polyprotein 1ab modulators, SARS-CoV-2 spike glycoprotein inhibitors, SARS-CoV-2 spike glycoprotein modulators, SARS-CoV-2 structural glycoprotein modulators, papain inhibitors, protease inhibitors, protease modulators, RNA polymerase inhibitors, RNA polymerase modulators, RNA-dependent RNA polymerase (RdRp) inhibitors, SARS coronavirus 3C protease like inhibitors, SARS-CoV-2 nsp14 methyltransferase enzyme inhibitor, 3CLpro/Mpro inhibitors, serine protease inhibitors, transmembrane serine protease 2 inhibitors, transmembrane serine protease 2 modulators, viral envelope protein inhibitors, viral protease inhibitors, viral protease modulators, viral protein target modulators, viral ribonuclease inhibitors, and viral structural protein modulators.

In some embodiments, the additional therapeutic agent is an entry inhibitor. For example, in some embodiments the additional therapeutic agent is an ACE2 inhibitor, a fusion inhibitor, or a protease inhibitor.

In some embodiments, the additional therapeutic agent is an angiotensin converting enzyme 2 inhibitor, such as SBK-001.

In some embodiments, the additional therapeutic agent is an angiotensin converting enzyme 2 modulator, such as neumifil or JN-2019.

In some embodiments, the additional therapeutic agent is an entry inhibitor such as MU-UNMC-1, or SAI-4.

In some embodiments, the additional therapeutic agent is an angiotensin converting enzyme 2 stimulator, such as alunacedase alfa.

In some embodiments, the additional therapeutic agent is an angiotensin II AT-2 receptor agonist, such as VP-01.

In some embodiments, the additional therapeutic agent is an ACE II receptor antagonist, such as DX-600.

In some embodiments, the additional therapeutic agent is an angiotensin II receptor modulator, such as TXA-127.

In some embodiments, the additional therapeutic agent is a transmembrane serine protease 2 modulator, such as BC-201, N-0385.

In some embodiments, the additional therapeutic agent is a viral envelope protein inhibitor, such as MXB-9 or MXB-004.

In some embodiments, the additional therapeutic agent is a RNAi agent such as ARO-COV or SNS-812.

In some embodiments, the additional therapeutic agent is a vaccine. For example, in some embodiments, the additional therapeutic agent is a DNA vaccine, RNA vaccine, live-attenuated vaccine, inactivated vaccine (i.e., inactivated SARS-CoV-2 vaccine), therapeutic vaccine, prophylactic vaccine, protein-based vaccine, viral vector vaccine, cellular vaccine, or dendritic cell vaccine.

In some embodiments, the additional therapeutic agent is a vaccine such as tozinameran, NVX—CoV2373, elasomeran, KD-414, Ad26.COV2-S, Vaxzevria, SCB-2019, AKS-452, VLA-2001, HDT-301, S-268019, MVC-COV1901, mRNA-1273.214, mRNA-1273.213, mRNA-1273.222, NVX—CoV2515, Covaxin, BBIBP-CorV, GBP-510, mRNA-1273.351+mRNA-1273.617 (SARS-CoV-2 multivalent mRNA vaccine, COVID-19), Ad5-nCoV, Omicron-based COVID-19 vaccine (mRNA vaccine, COVID-19), mRNA-1073, mRNA-1273.214, mRNA-1230, mRNA-1283, Omicron-based COVID-19 vaccine, SARS-CoV-2 Protein Subunit Recombinant Vaccine, Sputnik M, ZyCoV-D, COVID-19 XWG-03, mRNA-1273.529, mRNA-1010, CoronaVac, AZD-2816, Sputnik V, inactivated SARS-CoV-2 vaccine (Vero cell, COVID-19), DS-5670, PHH-1V, INO-4800, UB-612, coronavirus vaccine (whole-virion, inactivated/purified), ReCOV, MT-2766, ARCT-154, SP-0253, CORBEVAX, mRNA-1273.211, ZF-2001, Sputnik Light, recombinant protein vaccine (COVID-19/SARS-CoV-2 infection), VSV vector-based vaccine targeting spike glycoprotein (COVID-19), VLA-2101, GRT-R912, GRAd-COV2, VPM-1002, COViran Barekat, Ad5-nCoV-IH, ARCoV, Covax-19, recombinant SARS-CoV-2 vaccine (protein subunit/CHO cell, COVID-19), BBV-154, RAZI Cov Pars, COVID-19 vaccine (inactivated/Vero cells/intramuscular, SARS-CoV-2 infection), COVID-19 vaccine (inactivated, Vero cells/intramuscular), BNT-162b2s01, BNT-162b4, BNT-162b5, BNT-162b2 Omi, BNT-162b2 bivalent, CIGB-66, mRNA-1273.617, Mycobacterium w, ERUCOV-VAC, AG-0301-COVID19, fakhravac, AV-COVID-19, peptide vaccine (COVID-19), Nanocovax, SARS-CoV-2 vaccine (inactivated/Vero cells/intramuscular, COVID-19), QAZCOVID-IN, 5-875670 nasal vaccine, VTP-500, or BNT162b5.

In some embodiments, the additional therapeutic agent is a protease inhibitor. For example, in some embodiments the additional therapeutic agent is a 3C-like cysteine protease inhibitor (3CLpro, also called Main protease, Mpro), a papain-like protease inhibitor (PLpro), serine protease inhibitor, or transmembrane serine protease 2 inhibitor (TMPRSS2).

In some embodiments, the additional therapeutic agent is a 3CLpro/Mpro inhibitor, such as ABBV-903, AB-343, CDI-873, GC-373, GC-376, pomotrelvir (PBI-0451), UCI-1, bofutrelvir (FB-2001, DC-402234), DC-402267, GDI-4405, HS-10517, RAY-1216, MPI-8, SH-879, SH-580, EDP-235, VV-993, CDI-988, MI-30, nirmatrelvir, ensitrelvir, ASC-11, ASC-11+ritonavir, EDDC-2214, SIM-0417, PF-07817883, simnotrelvir, simnotrelvir+ritonavir, SYH-2055, ISM-3312, CDI-45205, LHP-803 (COR-803), ALG-097111, TJC-642, CVD-0013943, olgotrelvir (STI-1558), eravacycline, cynarine, WPV-01, or prexasertib.

In some embodiments, the additional therapeutic agent is a papain-like protease inhibitor (PLpro), such as SBFM-PL4 or GRL-0617.

In some embodiments, the additional therapeutic agent is a SARS-CoV-2 helicase Nsp13 inhibitor, such as EIS-4363.

In some embodiments, the additional therapeutic agent is a SARS-CoV-2 helicase Nsp14 inhibitor, such as TO-507.

In some embodiments, the additional therapeutic agent is a SARS-CoV-2 spike (S) and protease modulator, such as ENU-200.

In some embodiments, the additional therapeutic agent is a protease inhibitor, such as ALG-097558 or MRX-18.

In some embodiments, the additional therapeutic agent is a serine protease inhibitor, such as upamostat, nafamostat, camostat mesylate, nafamostat mesylate, or camostat.

In some embodiments, the additional therapeutic agent is a 3CLpro/transmembrane serine protease 2 inhibitor, such as SNB-01 (pentarlandir) or SNB-02.

In some embodiments, the additional therapeutic agent is a viral protease inhibitor, such as Pan-Corona, Cov-X, or bepridil.

In some embodiments, the additional therapeutic agent is an RNA polymerase inhibitor. For example, in some embodiments, the additional therapeutic agent is an RNA polymerase inhibitor, or an RNA-dependent RNA polymerase (RdRp) inhibitor.

In some embodiments, the additional therapeutic agent is an RNA-dependent RNA polymerase (RdRp) inhibitor, such as remdesivir, NV—CoV-2, NV—CoV-2-R, NV—CoV-1 encapsulated remdesivir, CMX-8521, GS-621763, GS-5245, GS-441524, DEP remdesivir, ATV-006, deuremidevir (VV-116), LGN-20, CMX-521, SHEN-26, MB-905, and compounds disclosed in WO2022142477, WO2021213288, WO2022047065.

In some embodiments, the additional therapeutic agent is an RNA polymerase inhibitor, such as molnupiravir (EIDD-2801), favipiravir, bemnifosbuvir, sofosbuvir, ASC-10, or galidesivir.

In some embodiments, the additional therapeutic agent is viral entry inhibitor, such as brilacidin.

In some embodiments, the additional therapeutic agent is an antibody that binds to a coronavirus, for example an antibody that binds to SARS or MERS.

In some embodiments, the additional therapeutic agent is an antibody, for example a monoclonal antibody. For example, the additional therapeutic agent is an antibody against SARS-CoV-2, neutralizing nanobodies, antibodies that target the SARS-CoV-2 spike protein, fusion proteins, multispecific antibodies, and antibodies that can neutralize SARS-CoV-2 (SARS-CoV-2 neutralizing antibodies).

In some embodiments, the additional therapeutic agent is an antibody that targets specific sites on ACE2. In some embodiments, the additional therapeutic agent is a polypeptide targeting SARS-CoV-2 spike protein (S-protein).

In some embodiments, the additional therapeutic agent is a SARS-CoV-2 virus antibody.

In some embodiments, the antibody is ABBV-47D11, COVI-GUARD (STI-1499), C144-LS+C135-LS, DIOS-202, DIOS-203, DIOS-301, DXP-604, JMB-2002, LY-CovMab, bamlanivimab (LY-CoV555), GIGA-2050, IBI-314, S309, SAB-185, etesevimab (CB6), COR-101, JS016, VNAR, VIR-7832 and/or sotrovimab (VIR-7831), casirivimab+imdevimab (REGN-COV2 or REGN10933+RGN10987), BAT2020, BAT2019, 47D11, YBSW-015, or PA-001.

In some embodiments, the additional therapeutic agent is STI-9199 (COVI-SHIELD), STI-9167 or AR-701 (AR-703 and AR-720).

In some embodiments, the additional therapeutic agent is BRII-196, BRII-198, ADG-10, adintrevimab (ADG-20), ABP-300, BA-7208, BI-767551, BHV-1200, CT-P63, JS-026, sotrovimab (GSK-4182136), tixagevimab+cilgavimab (AZD-7442), regdanvimab, SAB-301, AOD-01, plutavimab (COVI-AMG), 9MW-3311 (MW-33), DXP-593, BSVEQAb, anti-SARS-CoV-2 IgY, COVID-EIG, CSL-760, F-61, REGN-3048-3051, SARS-CoV-2 monoclonal antibodies (COVID-19, ADM-03820), enuzovimab (HFB-30132A), INM-005, SCTA01, TY-027, XAV-19, amubarvimab+romlusevimab, SCTA-01, bebtelovimab, beludavimab, I13I-0123, IGM-6268. FYB-207, FS-2101, RBT-0813, REGN-14256, REGN-14284, SPKM-001, XVR-011, TB202-3, TB181-36, TB339-031, LMN-301, LQ-050, COVAB-36, MAD-0004J08, STI-2099, TATX-03, TZLS-501, ZCB-11, AZD-3152, VYD-222, XVR-012, or ACV-200-17.

In some embodiments, the additional therapeutic agent is an engineered ACE-2-IgGl-Fc-fusion protein targeting SARS-Cov-2 RBD, such as EU-129, bivalent ACE2-IgG Fc null fusion protein (SI—F019).

In some embodiments, the additional therapeutic agent is an ACE2-Fc receptor fusion protein, such as HLX-71.

In some embodiments, the additional therapeutic agent is ensovibep.

In some embodiments, the additional therapeutic agent is SYZJ-001.

In some embodiments, the additional therapeutic agent is an HIV-1 protease inhibitor, such as ASC-09F (ASC-09+ritonavir) or lopinavir+ritonavir.

In some embodiments, the additional therapeutic agent is a non-nucleoside reverse transcriptase inhibitor, such as elsulfavirine.

In some embodiments, the additional therapeutic agent is a nucleoside reverse transcriptase inhibitor, such as azvudine.

In some embodiments, the additional therapeutic agent is Abbv-990, ABBV-903, 2b-11, 5-aminolevulinic phosphoric acid, AGP-600, AGM-380, AIP-502, ALG-150150, BAT-2022, NED-260, burfiralimab, ALG-097431, bardoxolone, BW—PS-119, clofoctol, CR-405, delcetravir, D4-102-01, D4-102-02, ESFAM-289, ENOB-CV-01, ENOB-CV-11, EIS-10700, EV-300, beta-521, GEA-001, SIM-0417, molnupiravir, Pan-Corona, Tollovir, nirmatrelvir+ritonavir (Paxlovid®), JTBC-00201, favipiravir, favipiravir+cathepsin inhibitor (TNX-3900), GC-376, upamostat, LeSoleil-01, LeSoleil-02+, benfovir, VV-116, VV-993, SNB-01, EDP-235, Cov-X, ensitrelvir, MPI-8, masitinib, ALG-097558, ASC-11, PBI-0451, nafamostat, nafamostat mesylate, CDI-45205, LHP-803 (COR-803), ALG-097111, BC-201, SH-879, CDI-873, CDI-988, remdesivir, NV—CoV-2-R, NV—CoV-1 encapsulated remdesivir, NA-831+remdesivir, DEP remdesivir, GS-621763, GS-5245, GLS-5310, bemnifosbuvir, QLS-1128, ASC-10, SBFM-PL4, camostat mesylate, UCI-1, FB-2001 (DC-402234), ebselen, SH-580, LeSoleil-01, LeSoleil-02+, MRX-18, MXB-9, MI-09, MI-30, SNB-02, SJP-002C, TJC-642, ENU-200, CVD-0013943, GS-441524, bepridil, MXB-004, eravacycline, GRL-0617, GST-HG171, GST-HG171+ritonavir, camostat, GC-373, KD-1, nitazoxanide, cynarine, prexasertib, PL-M, RAY-1216, SACT-COVID-19, MP-18, EIDD-1931, EDDC-2214, nitric oxide, apabetalone, AnQlar, SBK-001, LQ-050, CG-SpikeDown, bamlanivimab, JTBC-00101, HLX-71, HT-002, HY-209, HY-3000, HSC-1553, FYB-207, ensovibep, SYZJ-001, EU-129, neumifil, JN-2019 (KG-2019), LCB-99, AR-701, vostesyl, PLM-402, PJS-539, CTB-ACE2, TB181-36, TB202-3, ABP-300, XVR-011, MSP-008-22, MU-UNMC-1, MU-UNMC-2, MIC-1930, MLT-103, Mpro inhibitors (Anixa Biosciences), PBF-4554, alunacedase alfa, VP-01, TRV-027, DX-600, TXA-127, NVX—CoV2515, raphamin, RCYM-002, RCYM-003, riamilovir, SARS-Cov-2 PL pro inhibitor (Enanta), SBP-502, SM-4, STB-R040, THY-01, tozinameran, elasomeran, Ad5-nCoV, BBIBP-CorV, CoronaVac, MVC-COV1901, NVX—CoV2373, sotrovimab, Sputnik V, TEE-001, Tyme-19, Vaxzevria, XW-001, ZF-2001, or ZyCoV-D.

It is also possible to combine any compound of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds of the invention before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect, which is greater than the predicted purely additive effects of the individual compounds of the combination.

### IX. Examples

### Example 1: O1-[[(2R,3S,4R,5R)-5-(4-aminopyrrolo[2,1-f][1,2,4]triazin-7-yl)-5-cyano-3,4-dihydroxy-tetrahydrofuran-2-yl]methyl]O3-methyl indole-1,3-dicarboxylate (Compound 1)

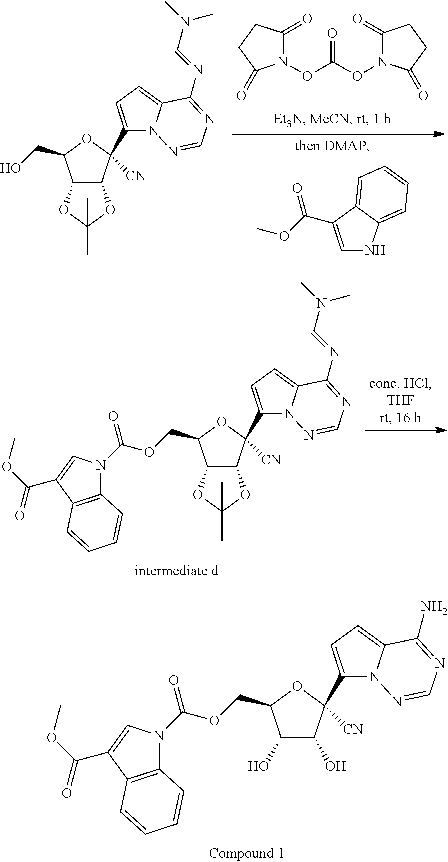

A stirred suspension of N′-[7-[(4R,6R,6aR)-4-cyano-6-(hydroxymethyl)-2,2-dimethyl-6,6a-dihydro-3aH-furo[3,4-d][1,3]dioxol-4-yl]pyrrolo[2,1-f][1,2,4]triazin-4-yl]-N,N-dimethyl-formamidine (200 mg, 0.52 mmol) and bis(2,5-dioxopyrrolidin-1-yl) carbonate (265 mg, 1.04 mmol) in acetonitrile (2 mL) at room temperature was treated with triethylamine (0.29 mL, 2.07 mmol), and was stirred for additional 1 hour. The resulting homogenous reaction mixture was treated with methyl 1H-indole-3-carboxylate (181 mg, 1.04 mmol) followed by 4-dimethylaminopyridine (63 mg, 0.52 mmol), and was stirred for 16 hours. The reaction mixture was concentrated to afford crude intermediate d, which was used directly in next step without further purification.

A solution of crude intermediate d in THE (3 mL) was treated with concentrated HCl (0.43 mL, 5.18 mmol) and was stirred for 16 h. The reaction mixture was basified with saturated aqueous sodium bicarbonate, then was extracted with EtOAc (3×10 mL). The combined organic layers were dried over MgSO₄, concentrated, and the resulting crude residue was purified by flash column chromatography using dichloromethane and methanol as eluents to afford Compound 1. 1H NMR (400 MHz, DMSO-d6) δ 8.20 (s, 1H), 8.17-8.05 (m, 2H), 7.81 (s, 3H), 7.48-7.34 (m, 2H), 6.87-6.80 (m, 2H), 6.37 (d, J=6.0 Hz, 1H), 5.51 (d, J=5.6 Hz, 1H), 4.87-4.74 (m, 2H), 4.62 (dd, J=12.0, 5.0 Hz, 1H), 4.45 (ddd, J=7.9, 5.0, 3.0 Hz, 1H), 4.19 (q, J=5.7 Hz, 1H), 3.88 (s, 3H). LCMS: MS m/z: 493.1 (M+1).

### Example 2: O1-[[(2R,3S,4R,5R)-5-(4-aminopyrrolo[2,1-f][1,2,4]triazin-7-yl)-5-cyano-3,4-dihydroxy-tetrahydrofuran-2-yl]methyl]O3-tetrahydropyran-4-yl indole-1,3-dicarboxylate (Compound 2)

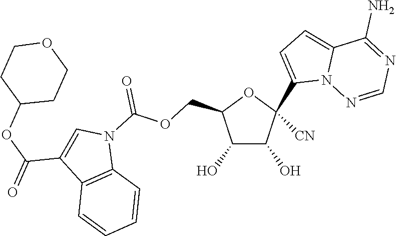 [SMILES: N#C[C@@]1(c2ccc3c(N)ncnn23)O[C@H](COC(=O)n2cc(C(=O)OC3CCOCC3)c3ccccc32)[C@@H](O)[C@H]1O]

Compound 2 was synthesized in manner similar to Compound 1 in Example 1, replacing methyl 1H-indole-3-carboxylate with tetrahydropyran-4-yl 1H-indole-3-carboxylate. 1H NMR (400 MHz, DMSO-d6) δ 8.24 (s, 1H), 8.19-8.03 (m, 2H), 7.98-7.73 (m, 3H), 7.48-7.34 (m, 2H), 6.92-6.80 (m, 2H), 6.37 (d, J=6.1 Hz, 1H), 5.61-5.49 (m, 1H), 5.22-5.14 (m, 1H), 4.88-4.76 (m, 2H), 4.64 (dd, J=12.0, 5.3 Hz, 1H), 4.51-4.43 (m, 1H), 4.24-4.14 (m, 1H), 3.92-3.81 (m, 2H), 3.61-3.49 (m, 2H), 2.04-1.95 (m, 2H), 1.80-1.64 (m, 2H). LCMS: MS m/z: 563.1 (M+1).

### Synthesis of tetrahydropyran-4-yl 1H-indole-3-carboxylate

A stirred suspension of 1H-indole-3-carboxylic acid (500 mg, 3.1 mmol) and KOH (174 mg, 3.1 mmol) in DMF (18 mL) was heated at 80° C. for 3 hours, cooled to 50° C., then treated with 4-bromotetrahydropyran (0.38 mL, 3.4 mmol) and stirred for 16 h. The reaction mixture was cooled to room temperature and quenched by addition of water (18 mL), and was extracted with diethyl ether (3×40 mL). The combined organic layers were washed with water (3×40 mL), dried over MgSO₄, concentrated, and crude residue was purified by flash column chromatography using ethyl acetate and hexanes as eluents to give tetrahydropyran-4-yl 1H-indole-3-carboxylate.

### Example 3: O1-[[(2R,3S,4R,5R)-5-(4-aminopyrrolo[2,1-f][1,2,4]triazin-7-yl)-5-cyano-3,4-dihydroxy-tetrahydrofuran-2-yl]methyl]O3-methyl 6-methoxyindole-1,3-dicarboxylate (Compound 3)

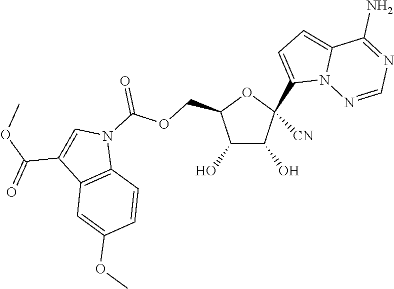 [SMILES: COC(=O)c1cn(C(=O)OC[C@H]2O[C@@](C#N)(c3ccc4c(N)ncnn34)[C@H](O)[C@@H]2O)c2ccc(OC)cc12]

Compound 3 was synthesized in manner similar to Compound 1 in Example 1, replacing methyl 1H-indole-3-carboxylate with methyl 6-methoxy-1H-indole-3-carboxylate. 1H NMR (400 MHz, DMSO-d6) δ 8.09 (s, 1H), 7.98-7.75 (m, 4H), 7.69 (d, J=2.3 Hz, 1H), 7.04 (dd, J=8.7, 2.4 Hz, 1H), 6.87-6.81 (m, 2H), 6.38 (d, J=6.1 Hz, 1H), 5.51 (d, J=5.6 Hz, 1H), 4.86-4.78 (m, 2H), 4.59 (dd, J=11.9, 5.4 Hz, 1H), 4.50-4.43 (m, 1H), 4.19 (q, J=5.7 Hz, 1H), 3.87 (s, 3H), 3.79 (s, 3H). LCMS: MS m/z: 523.1 (M+1).

### Example 4: [(2R,3S,4R,5R)-5-(4-aminopyrrolo[2,1-f][1,2,4]triazin-7-yl)-5-cyano-3,4-dihydroxy-tetrahydrofuran-2-yl]methyl (4S)-4-benzyl-2-oxo-oxazolidine-3-carboxylate (Compound 4)

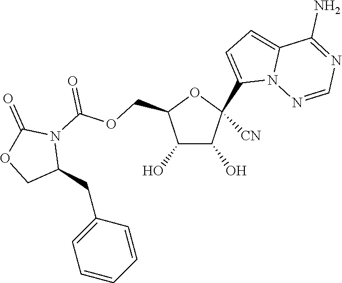 [SMILES: N#C[C@@]1(c2ccc3c(N)ncnn23)O[C@H](COC(=O)N2C(=O)OC[C@@H]2Cc2ccccc2)[C@@H](O)[C@H]1O]

Compound 4 was synthesized in manner similar to Compound 1 in Example 1, replacing methyl 1H-indole-3-carboxylate with (4S)-4-benzyloxazolidin-2-one. 1H NMR (400 MHz, DMSO-d6) δ 8.23-7.90 (m, 3H), 7.79 (s, 1H), 7.36-7.13 (m, 10O), 6.94 (d, J=4.6 Hz, 1H), 6.84 (d, J=4.6 Hz, 1H), 4.79 (d, J=5.1 Hz, 1H), 4.57-4.22 (m, 6H), 4.14 (dd, J=8.7, 2.9 Hz, 1H), 4.10-4.02 (m, 2H), 3.98 (dd, J=8.3, 5.4 Hz, 1H), 3.05-2.88 (m, 2H), 2.87-2.71 (m, 2H). LCMS: MS m/z: 495.1.

### Example 5: O3-[[(2R,3S,4R,5R)-5-(4-aminopyrrolo[2,1-f][1,2,4]triazin-7-yl)-5-cyano-3,4-dihydroxy-tetrahydrofuran-2-yl]methyl]04-methyl (4R)-2-oxooxazolidine-3,4-dicarboxylate (Compound 5)

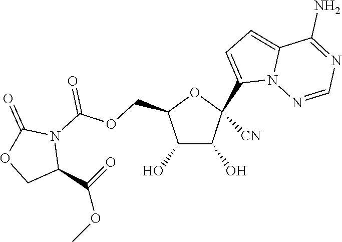 [SMILES: COC(=O)[C@H]1COC(=O)N1C(=O)OC[C@H]1O[C@@](C#N)(c2ccc3c(N)ncnn23)[C@H](O)[C@@H]1O]

Compound 5 was synthesized in manner similar to Compound 1 in Example 1, replacing methyl 1H-indole-3-carboxylate with methyl (4R)-2-oxooxazolidine-4-carboxylate. 1H NMR (400 MHz, DMSO-d6) δ 8.13-7.77 (m, 3H), 6.91 (d, J=4.6 Hz, 1H), 6.79 (d, J=4.5 Hz, 1H), 6.36 (d, J=6.1 Hz, 1H), 5.45 (d, J=5.6 Hz, 1H), 4.93 (dd, J=9.4, 3.4 Hz, 1H), 4.73 (t, J=5.6 Hz, 1H), 4.57 (t, J=9.2 Hz, 1H), 4.53-4.41 (m, 2H), 4.41-4.30 (m, 1H), 4.29-4.19 (m, 1H), 4.09-3.93 (m, 1H), 3.70 (s, 3H). LCMS: MS m/z: 463.1 (M+1).

### Example 6: ((((2R,3S,4R,5R)-5-(4-aminopyrrolo[2,1-f][1,2,4]triazin-7-yl)-5-cyano-3,4-dihydroxytetrahydrofuran-2-yl)methoxy)carbonyl)-L-valine (Compound 6)

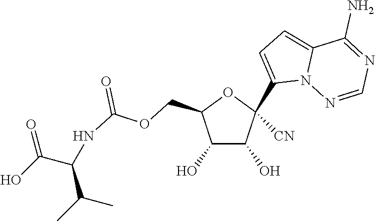 [SMILES: CC(C)[C@H](NC(=O)OC[C@H]1O[C@@](C#N)(c2ccc3c(N)ncnn23)[C@H](O)[C@@H]1O)C(=O)O]

Compound 6 was synthesized in manner similar to Compound 1 in Example 1, replacing methyl 1H-indole-3-carboxylate with L-valine. 1H NMR (400 MHz, DMSO-d6) δ 8.13-7.73 (m, 3H), 6.97 (d, J=4.6 Hz, 1H), 6.83 (d, J=4.6 Hz, 1H), 6.23 (d, J=7.9 Hz, 1H), 4.69 (d, J=5.0 Hz, 1H), 4.32-4.14 (m, 1H), 4.06 (dd, J=11.1, 6.0 Hz, 1H), 3.91 (s, 1H), 3.55 (dd, J=7.5, 4.1 Hz, 1H), 2.04 (s, 1H), 0.89-0.70 (m, 6H). LCMS: MS m/z: 435.1 (M+1).

### Example 7: isopropyl-((((2R,3S,4R,5R)-5-(4-aminopyrrolo[2,1-f][1,2,4]triazin-7-yl)-5-cyano-3,4-dihydroxytetrahydrofuran-2-yl)methoxy)carbonyl)-L-valinate (Compound 7)

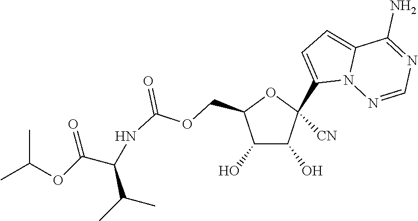 [SMILES: CC(C)OC(=O)[C@@H](NC(=O)OC[C@H]1O[C@@](C#N)(c2ccc3c(N)ncnn23)[C@H](O)[C@@H]1O)C(C)C]

Compound 7 was synthesized in manner similar to Compound 1 in Example 1, replacing methyl 1H-indole-3-carboxylate with L-valine isopropyl ester. 1H NMR (400 MHz, DMSO-d6) δ 8.02-7.81 (m, 3H), 7.63 (d, J=8.1 Hz, 1H), 6.92 (d, J=4.6 Hz, 1H), 6.83 (d, J=4.5 Hz, 1H), 6.28 (d, J=6.1 Hz, 1H), 5.38 (d, J=5.8 Hz, 1H), 5.00-4.87 (m, 1H), 4.67 (t, J=5.5 Hz, 1H), 4.34 (dd, J=12.0, 2.7 Hz, 1H), 4.21 (td, J=6.9, 2.7 Hz, 1H), 4.08 (dd, J=12.0, 7.1 Hz, 1H), 3.92-3.79 (m, 2H), 2.10-1.97 (m, 1H), 1.27-1.15 (m, 6H), 1.14-1.08 (m, 1H), 0.95-0.81 (m, 6H). LCMS: MS m/z: 477.2 (M+1).

### Example 8: [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate (Compound 8)

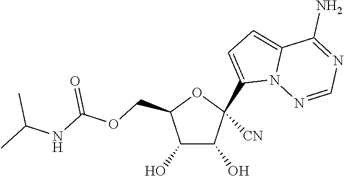 [SMILES: CC(C)NC(=O)OC[C@H]1O[C@@](C#N)(c2ccc3c(N)ncnn23)[C@H](O)[C@@H]1O]

Compound 8 was synthesized in manner similar to Compound 1 in Example 1, replacing methyl 1H-indole-3-carboxylate with propan-2-amine. 1H NMR (400 MHz, DMSO-d6) δ 8.56-8.37 (m, 1H), 8.34-8.16 (m, 1H), 8.03 (s, 1H), 7.16 (d, J=7.7 Hz, 1H), 7.05 (d, J=4.5 Hz, 1H), 6.90-6.80 (m, 1H), 4.65 (d, J=5.0 Hz, 1H), 4.34-4.27 (m, 1H), 4.24-4.16 (m, 2H), 4.06-3.99 (m, 1H), 3.93-3.87 (m, 1H), 1.28 (d, J=7.0 Hz, 3H), 1.01 (d, J=6.5 Hz, 3H). LCMS: MS m/z: 377.1 (M+1).

### Example 9: [(2R,3S,4R,5R)-5-(4-aminopyrrolo[2,1-f][1,2,4]triazin-7-yl)-5-cyano-3,4-dihydroxy-tetrahydrofuran-2-yl]methyl azocane-1-carboxylate (Compound 9)

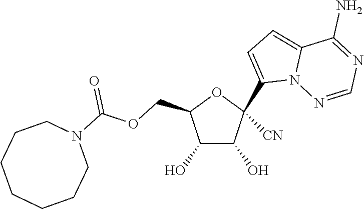 [SMILES: N#C[C@@]1(c2ccc3c(N)ncnn23)O[C@H](COC(=O)N2CCCCCCC2)[C@@H](O)[C@H]1O]

Compound 9 was synthesized in manner similar to Compound 1 in Example 1, replacing methyl 1H-indole-3-carboxylate with azocane. 1H NMR (400 MHz, DMSO-d6) δ 7.99-7.81 (m, 3H), 6.91 (d, J=4.5 Hz, 1H), 6.79 (d, J=4.5 Hz, 1H), 6.29 (d, J=6.0 Hz, 1H), 5.36 (d, J=5.8 Hz, 1H), 4.73-4.67 (m, 1H), 4.32-4.14 (m, 3H), 3.98 (q, J=5.8 Hz, 1H), 3.32-3.26 (m, 1H), 3.26-3.18 (m, 2H), 1.63-1.38 (m, 10H). LCMS: MS m/z: 431.1 (M+1).

### Example 10: O1-[[(2R,3S,4R,5R)-5-(4-aminopyrrolo[2,1-f][1,2,4]triazin-7-yl)-5-cyano-3,4-dihydroxy-tetrahydrofuran-2-yl]methyl]O2-ethyl pyrrole-1,2-dicarboxylate (Compound 10)

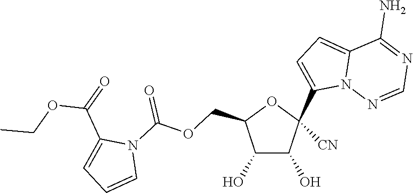 [SMILES: CCOC(=O)c1cccn1C(=O)OC[C@H]1O[C@@](C#N)(c2ccc3c(N)ncnn23)[C@H](O)[C@@H]1O]

Compound 10 was synthesized in manner similar to Compound 1 in Example 1, replacing methyl 1H-indole-3-carboxylate with ethyl 1H-pyrrole-2-carboxylate. 1H NMR (400 MHz, DMSO-d6) δ 8.01-7.80 (m, 3H), 7.38 (dd, J=3.1, 1.6 Hz, 1H), 6.92 (dd, J=3.5, 1.7 Hz, 1H), 6.87 (d, J=4.5 Hz, 1H), 6.78 (d, J=4.5 Hz, 1H), 6.35 (d, J=6.1 Hz, 1H), 6.31 (t, J=3.3 Hz, 1H), 5.47 (d, J=5.8 Hz, 1H), 4.81-4.73 (m, 1H), 4.64 (dd, J=12.0, 2.9 Hz, 1H), 4.49 (dd, J=12.0, 5.7 Hz, 1H), 4.35 (td, J=6.2, 2.8 Hz, 1H), 4.19 (qd, J=7.1, 1.0 Hz, 2H), 4.10-4.01 (m, 1H), 1.22 (t, J=7.1 Hz, 3H). LCMS: MS m/z: 457.1 (M+1).

### Example 11: [(2R,3S,4R,5R)-5-(4-aminopyrrolo[2,1-f][1,2,4]triazin-7-yl)-5-cyano-3,4-dihydroxy-tetrahydrofuran-2-yl]methyl 1,1-dioxo-1,4-thiazinane-4-carboxylate (Compound 11)

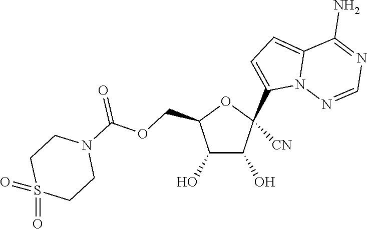 [SMILES: N#C[C@@]1(c2ccc3c(N)ncnn23)O[C@H](COC(=O)N2CCS(=O)(=O)CC2)[C@@H](O)[C@H]1O]

Compound 11 was synthesized in manner similar to Compound 1 in Example 1, replacing methyl 1H-indole-3-carboxylate with 1,4-thiazinane 1,1-dioxide. 1H NMR (400 MHz, DMSO-d6) δ 8.03-7.75 (m, 3H), 6.95-6.89 (m, 1H), 6.86-6.77 (m, 1H), 6.34 (d, J=6.0 Hz, 1H), 5.39 (t, J=5.9 Hz, 1H), 4.76-4.66 (m, 1H), 4.40-4.15 (m, 3H), 3.99 (qd, J=6.1, 3.0 Hz, 1H), 3.85-3.65 (m, 2H), 3.15 (s, 4H), 3.06 (dd, J=7.0, 3.5 Hz, 1H), 3.00-2.92 (m, 1H). LCMS: MS m/z: 453.1 (M+1).

### Example 12: [(2R,3S,4R,5R)-5-(4-aminopyrrolo[2,1-f][1,2,4]triazin-7-yl)-5-cyano-3,4-dihydroxy-tetrahydrofuran-2-yl]methyl isoxazolidine-2-carboxylate (Compound 12)

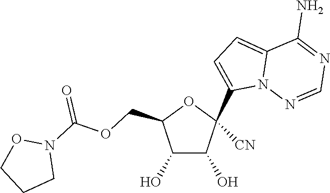 [SMILES: N#C[C@@]1(c2ccc3c(N)ncnn23)O[C@H](COC(=O)N2CCCO2)[C@@H](O)[C@H]1O]

Compound 12 was synthesized in manner similar to Compound 1 in Example 1, replacing methyl 1H-indole-3-carboxylate with isoxazolidine hydrochloride. 1H NMR (400 MHz, DMSO-d6) δ 8.05-7.79 (m, 3H), 6.91 (d, J=4.5 Hz, 1H), 6.84 (d, J=4.6 Hz, 1H), 6.34 (dd, J=6.0, 3.8 Hz, 1H), 5.39 (d, J=5.8 Hz, 1H), 4.67 (dd, J=6.0, 4.8 Hz, 1H), 4.40-4.32 (m, 1H), 4.29-4.20 (m, 2H), 4.00-3.92 (m, 1H), 3.83-3.76 (m, 2H), 3.57-3.48 (m, 2H), 2.21-2.11 (m, 2H). LCMS: MS m/z: 391.1 (M+1).

### Example 13: [(2R,3S,4R,5R)-5-(4-aminopyrrolo[2,1-f][1,2,4]triazin-7-yl)-5-cyano-3,4-dihydroxy-tetrahydrofuran-2-yl]methyl piperidine-1-carboxylate (Compound 13)

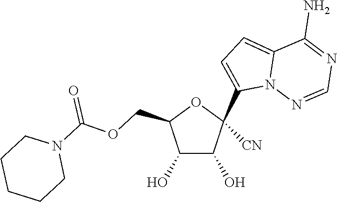 [SMILES: N#C[C@@]1(c2ccc3c(N)ncnn23)O[C@H](COC(=O)N2CCCCC2)[C@@H](O)[C@H]1O]

Compound 13 was synthesized in manner similar to Compound 1 in Example 1, replacing methyl 1H-indole-3-carboxylate with piperidine. 1H NMR (400 MHz, DMSO-d6) δ 7.94 (s, 2H), 6.93 (d, J=4.5 Hz, 1H), 6.78 (d, J=4.5 Hz, 1H), 4.67 (d, J=4.8 Hz, 1H), 4.31 (dd, J=12.1, 2.8 Hz, 1H), 4.26-4.18 (m, 1H), 4.12 (dd, J=12.1, 4.6 Hz, 1H), 4.00-3.92 (m, 1H), 3.27 (d, J=6.7 Hz, 3H), 1.65-1.33 (m, 6H). LCMS: MS m/z: 403.1 (M+1).

### Example 14: [(2R,3S,4R,5R)-5-(4-aminopyrrolo[2,1-f][1,2,4]triazin-7-yl)-5-cyano-3,4-dihydroxy-tetrahydrofuran-2-yl]methyl propylsulfanylformate (Compound 14)

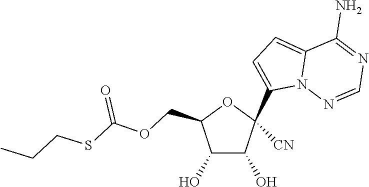 [SMILES: CCCSC(=O)OC[C@H]1O[C@@](C#N)(c2ccc3c(N)ncnn23)[C@H](O)[C@@H]1O]

Compound 14 was synthesized in manner similar to Compound 1 in Example 1, replacing methyl 1H-indole-3-carboxylate with propane-1-thiol. 1H NMR (400 MHz, DMSO-d6) δ 8.03-7.80 (m, 3H), 6.92 (d, J=4.5 Hz, 1H), 6.79 (d, J=4.5 Hz, 1H), 6.36 (d, J=6.0 Hz, 1H), 5.42 (d, J=5.8 Hz, 1H), 4.68 (dd, J=6.0, 4.9 Hz, 1H), 4.53 (dd, J=12.0, 2.8 Hz, 1H), 4.34 (dd, J=12.0, 5.5 Hz, 1H), 4.24 (ddd, J=8.3, 5.5, 2.7 Hz, 1H), 3.97-3.88 (m, 1H), 2.83 (td, J=7.0, 1.1 Hz, 2H), 1.59 (q, J=7.2 Hz, 2H), 0.92 (t, J=7.3 Hz, 3H). LCMS: MS m/z: 394.1 (M+1).

### Example 15: [(2R,3S,4R,5R)-5-(4-aminopyrrolo[2,1-f][1,2,4]triazin-7-yl)-5-cyano-3,4-dihydroxy-tetrahydrofuran-2-yl]methyl ethylsulfanylformate (Compound 15)

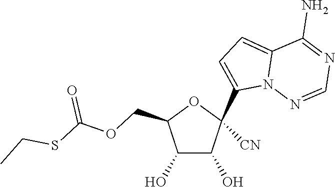 [SMILES: CCSC(=O)OC[C@H]1O[C@@](C#N)(c2ccc3c(N)ncnn23)[C@H](O)[C@@H]1O]

Compound 15 was synthesized in manner similar to Compound 1 in Example 1, replacing methyl 1H-indole-3-carboxylate with ethanethiol. 1H NMR (400 MHz, DMSO-d6) δ 8.02-7.80 (m, 3H), 6.91 (d, J=4.5 Hz, 1H), 6.79 (d, J=4.6 Hz, 1H), 6.35 (d, J=6.0 Hz, 1H), 5.42 (d, J=5.9 Hz, 1H), 4.74-4.62 (m, 1H), 4.53 (dd, J=12.1, 2.8 Hz, 1H), 4.34 (dd, J=12.0, 5.7 Hz, 1H), 4.29-4.19 (m, 1H), 4.19-4.10 (m, OH), 4.02-3.92 (m, 1H), 2.85 (q, J=7.3 Hz, 2H), 1.28-1.20 (m, 3H). LCMS: MS m/z: 380.0 (M+1).

### Example 16: [(2R,3S,4R,5R)-5-(4-aminopyrrolo[2,1-f][1,2,4]triazin-7-yl)-5-cyano-3,4-dihydroxy-tetrahydrofuran-2-yl]methyl cyclopentylsulfanylformate (Compound 16)

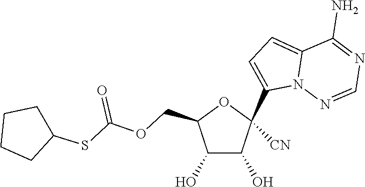 [SMILES: N#C[C@@]1(c2ccc3c(N)ncnn23)O[C@H](COC(=O)SC2CCCC2)[C@@H](O)[C@H]1O]

Compound 16 was synthesized in manner similar to Compound 1 in Example 1, replacing methyl 1H-indole-3-carboxylate with cyclopentanethiol. 1H NMR (400 MHz, DMSO-d6) δ 8.02-7.81 (m, 3H), 6.92 (d, J=4.5 Hz, 1H), 6.79 (d, J=4.5 Hz, 1H), 6.35 (d, J=6.0 Hz, 1H), 5.42 (d, J=5.8 Hz, 1H), 4.72-4.64 (m, 1H), 4.53 (dd, J=12.1, 2.8 Hz, 1H), 4.32 (dd, J=12.0, 5.4 Hz, 1H), 4.27-4.19 (m, 1H), 3.93 (q, J=5.9 Hz, 1H), 3.62-3.49 (m, 1H), 2.13-1.97 (m, 4H), 1.74-1.44 (m, 4H). LCMS: MS m/z: 420.1 (M+1).

### Example 17: [(2R,3S,4R,5R)-5-(4-aminopyrrolo[2,1-f][1,2,4]triazin-7-yl)-5-cyano-3,4-dihydroxy-tetrahydrofuran-2-yl]methyl tert-butylsulfanylformate (Compound 17)

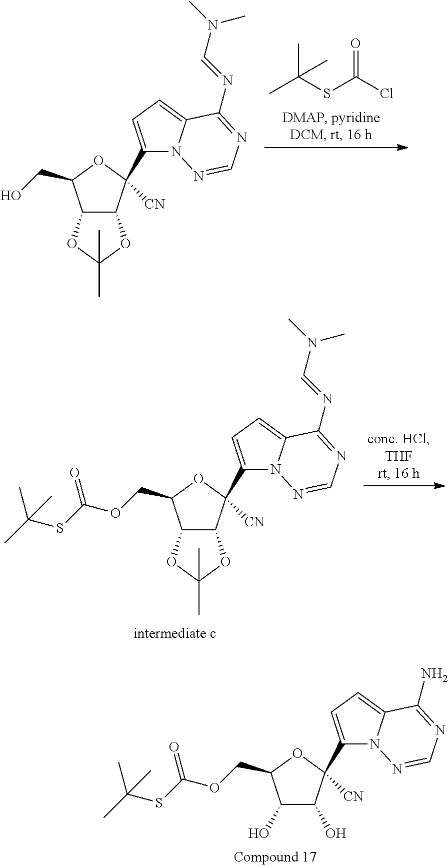

A solution of N′-[7-[(3aR,4R,6R,6aR)-4-cyano-6-(hydroxymethyl)-2,2-dimethyl-6,6a-dihydro-3aH-furo[3,4-d][1,3]dioxol-4-yl]pyrrolo[2,1-f][1,2,4]triazin-4-yl]-N,N-dimethyl-formamidine (150 mg, 0.39 mmol), 4-dimethylaminopyridine (47.4 mg, 0.39 mmol) and pyridine (0.09 mL, 1.16 mmol) in dichloromethane (4 mL) at room temperature was treated with S-tert-butyl chloromethanethioate (89 mg, 0.58 mmol), and was stirred for additional 16 h. The reaction mixture was quenched with water (4 mL), and extracted with dichloromethane (3×10 mL). The combined organic layers were dried over MgSO₄ then concentrated to afford crude intermediate c, which was used directly in next step without further purification.

A solution of crude intermediate c in THE (2.25 mL) at room temperature was treated with conc. HCl (0.48 mL, 5.82 mmol), and was stirred for additional 3.5 hours before quenched and basified with saturated aqueous sodium bicarbonate. The biphasic mixture was extracted with EtOAc (3×10 mL), and the combined organic layers were dried over MgSO₄ then concentrated. The resulting crude residue was purified by flash column chromatography using methanol and dichloromethane to afford Compound 17. 1H NMR (400 MHz, DMSO-d6) δ 8.07-7.74 (m, 3H), 6.92 (d, J=4.5 Hz, 1H), 6.79 (d, J=4.6 Hz, 1H), 6.35 (d, J=6.0 Hz, 1H), 5.41 (d, J=5.8 Hz, 1H), 4.74-4.61 (m, 1H), 4.52 (dd, J=11.8, 2.6 Hz, 1H), 4.32-4.15 (m, 2H), 3.92 (q, J=5.9 Hz, 1H), 1.44 (s, 9H). LCMS: MS m/z: 408.1 (M+1).

### Example 18: [(2R,3R,4R,5R)-4-acetoxy-5-(4-aminopyrrolo[2,1-f][1,2,4]triazin-7-yl)-5-cyano-2-(isopropylcarbamoyloxymethyl)tetrahydrofuran-3-yl]acetate (Compound 18)

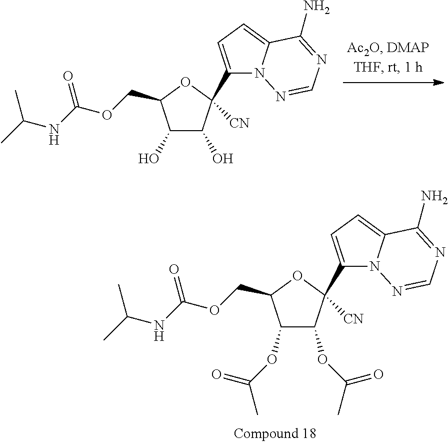 [SMILES: CC(C)NC(=O)OC[C@H]1O[C@@](C#N)(c2ccc3c(N)ncnn23)[C@H](O)[C@@H]1O ; CC(=O)O[C@H]1[C@@H](OC(C)=O)[C@](C#N)(c2ccc3c(N)ncnn23)O[C@@H]1COC(=O)NC(C)C]

A solution of [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate (150 mg, 0.31 mmol) and 4-dimethylaminopyridine (112 mg, 0.92 mmol) in THF (3 mL) at room temperature was treated with acetic anhydride (62.5 mg, 0.61 mmol), and was stirred for additional 1 hour. The reaction mixture was quenched by addition of saturated aqueous ammonium chloride (10 mL) and was extracted with EtOAc (3×10 mL). The combined organic layers were dried over MgSO₄, concentrated, and the resulting crude residue was purified by flash column chromatography using reverse phase C18 column and acetonitrile and water with 0.1% TFA as eluents to afford Compound 18. 1H NMR (400 MHz, DMSO-d6) δ 8.61-8.19 (m, 2H), 8.04 (s, 1H), 7.19 (d, J=7.8 Hz, 1H), 7.05 (d, J=4.6 Hz, 1H), 6.86 (d, J=4.6 Hz, 1H), 6.01 (d, J=6.0 Hz, 1H), 5.76 (s, 1H), 5.33 (t, J=5.6 Hz, 1H), 4.52 (d, J=4.3 Hz, 1H), 4.43-4.35 (m, 1H), 4.17-4.09 (m, 1H), 3.56 (h, J=6.7 Hz, 1H), 2.21-2.07 (m, 6H), 1.09-0.97 (m, 6H). LCMS: MS m/z: 461.1 (M+1).

### Example 19: [(2R,3R,4R,5R)-3,4-diacetoxy-5-(4-aminopyrrolo[2,1-f][1,2,4]triazin-7-yl)-5-cyano-tetrahydrofuran-2-yl]methyl azocane-1-carboxylate (Compound 19)

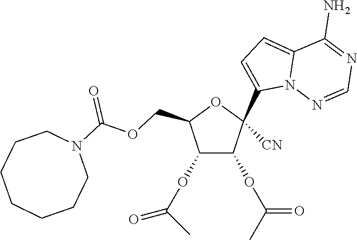 [SMILES: CC(=O)O[C@H]1[C@@H](OC(C)=O)[C@](C#N)(c2ccc3c(N)ncnn23)O[C@@H]1COC(=O)N1CCCCCCC1]

Compound 19 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl azocane-1-carboxylate. 1H NMR (400 MHz, DMSO-d6) δ 8.12-7.90 (m, 3H), 6.93 (d, J=4.6 Hz, 1H), 6.77 (d, J=4.6 Hz, 1H), 6.04 (d, J=5.9 Hz, 1H), 5.43 (dd, J=5.9, 4.3 Hz, 1H), 4.60 (q, J=3.9 Hz, 1H), 4.28 (qd, J=12.3, 3.7 Hz, 2H), 3.31-3.19 (m, 4H), 2.15-2.09 (m, 6H), 1.66-1.34 (m, 10H). LCMS: MS m/z: 515.0 (M+1).

### Example 20: O1-[[(2R,3R,4R,5R)-3,4-diacetoxy-5-(4-aminopyrrolo[2,1-f][1,2,4]triazin-7-yl)-5-cyano-tetrahydrofuran-2-yl]methyl]O2-ethyl pyrrole-1,2-dicarboxylate (Compound 20)

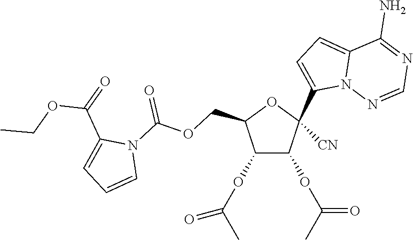 [SMILES: CCOC(=O)c1cccn1C(=O)OC[C@H]1O[C@@](C#N)(c2ccc3c(N)ncnn23)[C@H](OC(C)=O)[C@@H]1OC(C)=O]

Compound 20 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with 01-[[(2R,3S,4R,5R)-5-(4-aminopyrrolo[2,1-f][1,2,4]triazin-7-yl)-5-cyano-3,4-dihydroxy-tetrahydrofuran-2-yl]methyl]02-ethyl pyrrole-1,2-dicarboxylate. 1H NMR (400 MHz, DMSO-d6) δ 8.00 (d, J=26.4 Hz, 2H), 7.75 (s, 1H), 7.49-7.38 (m, 1H), 7.00-6.87 (m, 2H), 6.82 (d, J=4.6 Hz, 1H), 6.34 (t, J=3.4 Hz, 1H), 6.22 (d, J=6.1 Hz, 1H), 5.53 (t, J=5.3 Hz, 1H), 4.82-4.65 (m, 2H), 4.65-4.50 (m, 1H), 4.17 (tt, J=7.8, 4.0 Hz, 2H), 2.23-2.06 (m, 6H), 1.20 (t, J=7.1 Hz, 3H). LCMS: MS m/z: 540.9 (M+1).

### Example 21: [(2R,3R,4R,5R)-3,4-diacetoxy-5-(4-aminopyrrolo[2,1-f][1,2,4]triazin-7-yl)-5-cyano-tetrahydrofuran-2-yl]methyl piperidine-1-carboxylate (Compound 21)

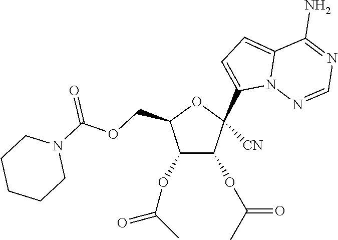 [SMILES: CC(=O)O[C@H]1[C@@H](OC(C)=O)[C@](C#N)(c2ccc3c(N)ncnn23)O[C@@H]1COC(=O)N1CCCCC1]

Compound 21 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl piperidine-1-carboxylate. 1H NMR (400 MHz, DMSO-d6) δ 8.10-7.87 (m, 3H), 6.94 (d, J=4.6 Hz, 1H), 6.76 (d, J=4.6 Hz, 1H), 6.03 (d, J=5.7 Hz, 1H), 5.41 (dd, J=5.7, 4.5 Hz, 1H), 4.61 (q, J=3.8 Hz, 1H), 4.40-4.15 (m, 2H), 3.30-3.19 (m, 4H), 2.16-2.10 (m, 6H), 1.55-1.46 (m, 2H), 1.45-1.30 (m, 4H). LCMS: MS m/z: 487.1 (M+1).

### Example 22: [(2R,3R,4R,5R)-4-acetoxy-5-(4-aminopyrrolo[2,1-f][1,2,4]triazin-7-yl)-5-cyano-2-(propylsulfanylcarbonyloxymethyl)tetrahydrofuran-3-yl]acetate (Compound 22)

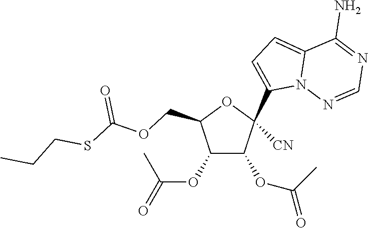 [SMILES: CCCSC(=O)OC[C@H]1O[C@@](C#N)(c2ccc3c(N)ncnn23)[C@H](OC(C)=O)[C@@H]1OC(C)=O]

Compound 22 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl propylsulfanylformate. 1H NMR (400 MHz, DMSO-d6) δ 8.45-8.14 (m, 2H), 8.01 (s, 1H), 7.02 (d, J=4.6 Hz, 1H), 6.82 (d, J=4.6 Hz, 1H), 6.05 (d, J=5.9 Hz, 1H), 5.39 (dd, J=5.9, 4.7 Hz, 1H), 4.71-4.53 (m, 2H), 4.43 (dd, J=12.1, 4.6 Hz, 1H), 2.81 (t, J=7.1 Hz, 2H), 2.15-2.09 (m, 6H), 1.56 (h, J=7.3 Hz, 2H), 0.90 (t, J=7.4 Hz, 3H). LCMS: MS m/z: 478.1 (M+1).

### Example 23: [(2R,3R,4R,5R)-4-acetoxy-5-(4-aminopyrrolo[2,1-f][1,2,4]triazin-7-yl)-5-cyano-2-(ethylsulfanylcarbonyloxymethyl)tetrahydrofuran-3-yl]acetate (Compound 23)

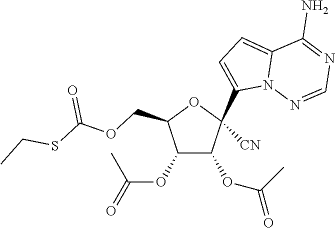 [SMILES: CCSC(=O)OC[C@H]1O[C@@](C#N)(c2ccc3c(N)ncnn23)[C@H](OC(C)=O)[C@@H]1OC(C)=O]

Compound 23 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl ethylsulfanylformate. 1H NMR (400 MHz, DMSO-d6) δ 8.37-8.08 (m, 2H), 7.99 (s, 1H), 7.00 (d, J=4.6 Hz, 1H), 6.82 (d, J=4.6 Hz, 1H), 6.05 (d, J=5.9 Hz, 1H), 5.39 (dd, J=5.9, 4.7 Hz, 1H), 4.68-4.51 (m, 2H), 4.44 (dd, J=12.1, 4.7 Hz, 1H), 2.83 (q, J=7.3 Hz, 2H), 1.21 (t, J=7.3 Hz, 3H). LCMS: MS m/z: 464.0 (M+1).

### Example 24: [(2R,3R,4R,5R)-4-acetoxy-5-(4-aminopyrrolo[2,1-f][1,2,4]triazin-7-yl)-2-(tert-butylsulfanylcarbonyloxymethyl)-5-cyano-tetrahydrofuran-3-yl]acetate (Compound 24)

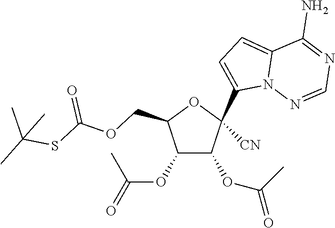 [SMILES: CC(=O)O[C@H]1[C@@H](OC(C)=O)[C@](C#N)(c2ccc3c(N)ncnn23)O[C@@H]1COC(=O)SC(C)(C)C]

Compound 24 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl tert-butylsulfanylformate. 1H NMR (400 MHz, DMSO-d6) δ 8.40-8.12 (m, 2H), 8.01 (s, 1H), 7.01 (d, J=4.6 Hz, 1H), 6.81 (d, J=4.6 Hz, 1H), 6.02 (d, J=5.9 Hz, 1H), 5.37 (dd, J=5.8, 4.7 Hz, 1H), 4.67-4.52 (m, 2H), 4.39 (dd, J=12.2, 4.7 Hz, 1H), 2.20-2.06 (m, 6H), 1.40 (s, 9H). LCMS: MS m/z: 492.0 (M+1).

### Example 25: [(2R,3R,4R,5R)-4-acetoxy-5-(4-aminopyrrolo[2,1-f][1,2,4]triazin-7-yl)-5-cyano-2-(cyclopentylsulfanylcarbonyloxymethyl)tetrahydrofuran-3-yl]acetate (Compound 25)

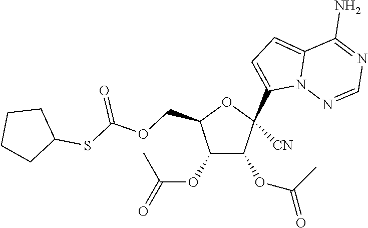 [SMILES: CC(=O)O[C@H]1[C@@H](OC(C)=O)[C@](C#N)(c2ccc3c(N)ncnn23)O[C@@H]1COC(=O)SC1CCCC1]

Compound 25 was synthesized in manner similar to Compound 18 in Example 18, replacing [(2R,3S,4R,5R)-5-(4-aminopyrrolo[2,1-f][1,2,4]triazin-7-yl)-5-cyano-3,4-dihydroxy-tetrahydrofuran-2-yl]methyl N-isopropylcarbamate with [(2R,3S,4R,5R)-5-(4-aminopyrrolo[2,1-f][1,2,4]triazin-7-yl)-5-cyano-3,4-dihydroxy-tetrahydrofuran-2-yl]methyl cyclopentylsulfanylformate. 1H NMR (400 MHz, Chloroform-d) δ 8.04 (s, 1H), 6.99 (d, J=4.7 Hz, 1H), 6.63 (d, J=4.7 Hz, 1H), 6.26 (d, J=5.9 Hz, 1H), 5.75-5.56 (m, 2H), 5.53-5.45 (m, 1H), 4.66-4.53 (m, 2H), 4.46 (dd, J=11.7, 4.2 Hz, 1H), 3.68-3.53 (m, 1H), 2.18-2.13 (m, 6H), 1.79-1.51 (m, 8H). LCMS: MS m/z: 504.1 (M+1).

### Example A: A549-hACE2 SARS-CoV2-NLuc 384-Well Assay

A549-hACE2 cell line was maintained in Dulbecco's Minimum Essential Medium (DMEM) (Corning, New York, NY, Cat #15-018CM) supplemented with 10% fetal bovine serum (FBS) (Hyclone, Logan, UT, Cat #SH30071-03), 1× Penicillin-Streptomycin-L-Glutamine (Corning, New York, NY, Cat #30-009-CI) and 10 μg/mL blasticidin (Life Technologies Corporation, Carlsbad, CA, Cat #A11139-03). Cells were passaged 2 times per week to maintain sub-confluent densities and were used for experiments at passage 5-20. SARS Coronavirus 2 recombinant with NanoLuc (SARS-CoV2-NLuc) was obtained from University of Texas Medical Branch (Galveston, TX). Viral replication was determined in A549-hACE2 cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well using an Echo acoustic dispenser (Labcyte, Sunnyvale, CA). A549-hACE2 cells were harvested and suspended in DMEM (supplemented with 2% FBS and 1× Penicillin-Streptomycin-L-Glutamine) and seeded to the pre-spotted assay plates at 10,000 cells per well in 30 μL. SARS-CoV2-NLuc virus was diluted in DMEM (supplemented with 2% FBS and 1× Penicillin-Streptomycin-L-Glutamine) at 350,000 Infectious Units (IU) per mL and 10 μL per well was added to the assay plates containing cells and compounds, for an MOI of 0.35. The assay plates were incubated for 2 days at 37° C. and 5% CO₂. At the end of incubation, Nano-Glo reagent (Promega, Madison, WI, Cat #N1150) was prepared. The assay plates and Nano-Glo reagent were equilibrated to room temperature for at least 15 minutes. 40 μL per well of Nano-Glo reagent was added and the plates were incubated at room temperature for 15 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing the viral replication by 50%.

### Remdesivir:

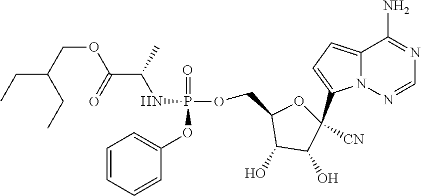 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

### Example B: NBBE RSV-Luc5 384-Well Assay

Normal Human Bronchial Epithelial (NHBE) cells were purchased from Lonza (Walkersville, MD Cat #CC2540) and maintained in BEGM Bronchial Epithelial Cell Growth Medium BulletKit (Lonza CC-3170).

Cells were thawed, expanded, and were used for experiments at passage 2. Respiratory syncytial virus recombinant with luciferase (RSV-Luc5) (>1×10⁷ Infectious Units/ml (IU/ml) determined by TCID₅₀) was purchased from Microbiologics (Saint Cloud, MN). Viral replication was determined in NHBE cells in the following manner.

Compounds are prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well. NHBE cells were harvested and suspended in BEGM Bronchial Epithelial Cell Growth Medium BulletKit and seeded to the pre-spotted assay plates at 5,000 cells per well in 30 μL. RSV-Luc5 virus was diluted in BEGM Bronchial Epithelial Cell Growth Medium BulletKit at 500,000 Infectious Units (IU) per mL and 10 μL per well was added to the assay plates containing cells and compounds, for an MOI of 1. The assay plates were incubated for 3 days at 37° C. and 5% CO₂. At the end of incubation, One-Glo reagent (Promega, Madison, WI, Cat #E6120) was prepared. The assay plates and One-Glo reagent were equilibrated to room temperature for at least 15 minutes. 40 μL per well of One-Glo reagent was added and the plates were incubated at room temperature for 15 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing the viral replication by 50%.

### Example C: CC50 MT4

Cytotoxicity of the compounds was determined in uninfected cells using the cell viability reagent in a similar fashion as described before for other cell types (Cihlar et al., Antimicrob Agents Chemother. 2008, 52(2):655-65). HEp-2 (1.5×103 cells/well) and MT-4 (2×103 cells/well) cells were plated in 384-well plates and incubated with the appropriate medium containing 3-fold serially diluted compound ranging from 15 nM to 100,000 nM. Cells were cultured for 4-5 days at 37° C. Following the incubation, the cells were allowed to equilibrate to 25° C., and cell viability was determined by adding Cell-Titer Glo viability reagent. The mixture was incubated for 10 min, and the luminescence signal was quantified using an Envision plate reader. Untreated cell and cells treated at 2 μM puromycin (Sigma, St. Louis, MO) serve as 10000 and 000 cell viability control, respectively. The percent of cell viability was calculated for each tested compound concentration relative to the 0% and 100% controls and the CC₅₀ value was determined by non-linear regression as a compound concentration reducing the cell viability by 500%.

TABLE C1

| | RSV NHBE | SARS-CoV2 | MT4 CC₅₀ |
|---|---|---|---|
| | EC₅₀ (nM) | EC₅₀ (nM) | (nM) |
| Compound 1 | 5586.9 | 4726.1 | >50000 |
| Compound 2 | 14130 | 5515.8 | >50000 |
| Compound 3 | 11265 | 5031.1 | >50000 |
| Compound 4 | >50000 | >50000 | >50000 |
| Compound 5 | 20075 | 20403 | >50000 |
| Compound 6 | >50000 | >50000 | >50000 |
| Compound 7 | >50000 | >50000 | >50000 |
| Compound 8 | >50000 | 31908 | >50000 |
| Compound 9 | 10303 | >50000 | >50000 |
| Compound 10 | 1521.2 | 5157.7 | >50000 |
| Compound 11 | >50000 | >50000 | >50000 |
| Compound 12 | 35319 | >50000 | >50000 |
| Compound 13 | >50000 | >50000 | >50000 |
| Compound 14 | 2624.7 | 11658 | >50000 |
| Compound 15 | 5652.9 | 19666 | >50000 |
| Compound 16 | 2807.3 | 8753.5 | 49929 |
| Compound 17 | 8111 | 50000 | 49782 |
| Compound 18 | >50000 | >50000 | >50000 |
| Compound 19 | 18011 | 31465 | >50000 |
| Compound 20 | 730.57 | 4275.4 | >50000 |
| Compound 21 | >50000 | >50000 | >50000 |
| Compound 22 | 3320.9 | 9833.2 | >50000 |
| Compound 23 | 3813.7 | 12433 | >50000 |
| Compound 24 | 19390 | >50000 | >50000 |
| Compound 25 | 1246 | 4018.2 | 32540 |

### Example D: GI S9 Stability—No Phenylmethylsulfonyl Fluoride (PMSF)

Duplicate aliquots of test compound or positive control substrate (GS-7340) were added to PMSF-free Intestinal S9 fractions diluted with 100 mM phosphate buffered saline, pH 7.4, to obtain a protein concentration of 1.0 mg/mL. The S9 metabolic reactions were initiated by the addition of the substrates to the S9 reaction mixture to a final concentration of 2 μM. At 0, 10, 20, 30, 60 and 120 min, 25 μL aliquots of the reaction mixture were transferred to plates containing 225 μl of IS/Q solution. After quenching, the plates were centrifuged at 3000×g for 30 minutes, and 150 μL aliquots of each supernatant were diluted with 150 μL water. Aliquots (10 μL) of the diluted supernatant were analyzed on a Thermo Q-Exactive mass spectrometer as described below.

### Example E: Plasma Stability

Duplicate aliquots of plasma were warmed to 37° C. and the metabolic reactions initiated by the addition of test compound (6 μL of 0.1 mM DMSO stock) or plasma stability standard (GS-7340) to obtain a final substrate concentration of 2 μM. At 0.05, 0.5, 1, 2, 3 and 4 hr, 25 μL aliquots of the reaction mixture were transferred to plates containing 225 μl of IS/Q quenching solution. After quenching, the plates were centrifuged at 3000×g for 30 minutes, and 150 μL supernatant was diluted with 150 μL water. Aliquots (10 μL) of the diluted supernatant were analyzed on a Thermo Q-Exactive mass spectrometer as described below.

### Example F: Hepatic S9 Stability

Duplicate aliquots of test compound or positive control substrate (GS-7340) were added to S9 stock diluted with 100 mM potassium phosphate buffer, pH 7.4, to obtain a protein concentration of 2.4 mg/mL. The S9 metabolic reactions were initiated by the addition of the substrates to the S9 reaction mixture to a final concentration of 2 μM. At 2, 12, 25, 45, 65 and 90 min, 25 μL aliquots of the reaction mixture were transferred to plates containing 225 μl of IS/Q solution. After quenching, the plates were centrifuged at 3000×g for 30 minutes, and 150 μL aliquots of each supernatant were diluted with 150 μL water. Aliquots (10 μL) of the diluted supernatant were analyzed on a Thermo Q-Exactive mass spectrometer as described below.

### Example G: Liquid Chromatography/Mass Spectroscopy Methods for S9 and Plasma Stability

Quantification of test compounds and controls was performed by analyte/internal standard peak area ratio (PAR) values measured on a Thermo Q-Exactive mass spectrometer coupled to a Dionex UltiMate 3000 HPLC with a Leap Technologies HTC PAL autosampler. The column used was a Thermo Hypersil GOLD (1.9 μm particle size, 2.1×50 mm). Mobile phase A consisted of 0.100 (v/v) formic acid in water. Mobile phase B consisted of 0.1% (v/v) formic acid in acetonitrile. Elution of analytes was achieved by a series of linear gradients of acetonitrile in water containing 0.1% (v/v) formic acid. The mass spectrometer was calibrated on a weekly basis and mass tolerance of 5 ppm was used.

TABLE G1

| | GIS9- | | |
|---|---|---|---|
| | HUMAN- | HEPS9-HUMAN- | STA Human |
| | NoPMSF T1/2 | T1/2 | Plasma 37 |
| Compound 1 | 123.06 | | 645.34 |
| Compound 2 | 475.93 | | 1055.4 |
| Compound 3 | 285.13 | | 100.42 |
| Compound 4 | 789 | | 253.23 |
| Compound 5 | 83.352 | 255.7 | 77.22 |
| Compound 6 | 792 | | 1584 |
| Compound 7 | 792 | 592 | 1303.4 |
| Compound 8 | 784 | 592 | |
| Compound 9 | 400.2 | 525.5 | 1584 |
| Compound 10 | 1.712 | 4.6 | 68.966 |
| Compound 11 | 779.31 | | 1584 |
| Compound 12 | 85.023 | | 938.47 |
| Compound 13 | 789 | 592 | 1584 |
| Compound 14 | 5.119 | | 495.99 |
| Compound 15 | 16.401 | | 451.78 |
| Compound 16 | 3.399 | 6.2 | |
| Compound 17 | 44.468 | | 1343.5 |
| Compound 18 | 0.581 | | 16.014 |
| Compound 19 | 0.764 | 2 | 14.703 |
| Compound 20 | 0.627 | 2 | 67.544 |
| Compound 21 | 0.673 | 2 | 11.334 |
| Compound 22 | 1.591 | 2 | |
| Compound 23 | 0.885 | 2 | |
| Compound 24 | 0.921 | 2 | |
| Compound 25 | 1.142 | 2 | 72.169 |

### Example H: CACO-2 Permeability

Pre-plated Caco-2 cells (clone C2BBel) were obtained from Sigma-Aldrich, Inc. (Atlanta, GA). Cell monolayers were grown to confluence on collagen-coated, microporous, polycarbonate membranes in 24-well transwell plates for 21 days. The permeability assay buffer in donor wells was Hanks' balanced salt solution (HBSS) containing 10 mM HEPES and 15 mM glucose at a pH of 6.5 containing 200 μM BNPP. The receiver wells used HBSS buffer containing 10 mM HEPES and 15 mM glucose at a pH of 7.4 and supplemented with 1% BSA. After an initial equilibration with transport buffer, TEER values were read to test membrane integrity. The experiment was started by the addition of buffers containing test compounds, 200 μl and 1000 μl in the apical and basolateral chamber, respectively. At 0- and 2-hour post dose, 10 μL was sampled from donor compartment and was immediately diluted in 190 μL of 20% methanol. At 1 and 2 hours post dose, 100 μl of solution was taken from the receiver compartments and was immediately diluted in 100 μl of 20% methanol. Removed buffer was replaced with fresh buffer and a correction was applied to all calculations for the removed material. Each compound was tested in 2 separate replicate wells for each condition. All samples were then extracted with 400 μl 100% acetonitrile with internal standard to precipitate protein. Cells were dosed on the apical or basolateral side to determine forward (A to B) and reverse (B to A) permeability. To test for non-specific binding and compound instability, the total amount of drug was quantitated at the end of the experiment and compared to the material present in the original dosing solution as a percent recovery. Formation of the parent compound (GS-441524; Reference Compound A) was monitored in assay wells dosed with the prodrug. Samples were analyzed by LC-MS/MS.

TABLE H1

| | DM Caco2 A to B | DM Caco2 B to A |
|---|---|---|
| Compound 1 | 0.69 | 12.34 |
| Compound 2 | 0.33 | 13.49 |
| Compound 3 | 0.43 | 10.2 |
| Compound 4 | 0.12 | 0.94 |
| Compound 5 | 0.04 | 0.08 |
| Compound 6 | 0.05 | 0.07 |
| Compound 7 | 0.115 | 6.245 |
| Compound 8 | 0.11 | 0.79 |
| Compound 9 | 0.47 | 6.67 |
| Compound 10 | 0.22 | 5.54 |
| Compound 11 | 0.08 | 0.11 |
| Compound 12 | 0.08 | 0.36 |
| Compound 13 | 0.3 | 6.39 |
| Compound 14 | 2.06 | 9.1 |
| Compound 15 | 1.51 | 7.08 |
| Compound 16 | | |
| Compound 17 | 3.05 | 15.63 |
| Compound 18 | 0.8 | 6.91 |
| Compound 19 | 6.33 | 13.32 |
| Compound 20 | 3.16 | 9.63 |
| Compound 21 | 3.99 | 19.68 |
| Compound 22 | | |
| Compound 23 | | |
| Compound 24 | | |
| Compound 25 | | |

### Example I: Thermodynamic Solubility in pH7 Buffered Solution

The aqueous solubility of compounds over a time of 24 hours was assessed. Solubility was determined at ambient temperature in a 50 mM phosphate buffered pH7 solution with 150 mM NaCl (to achieve isotonicity). Solids were added to the buffered solution in 1.5-mL Eppendorf tubes, vortexed for 1 minute, then agitated for 24 hours in an Eppendorf ThermoMixer C. To determine concentration in solution, the suspensions were centrifuged for 15 min at 15,000 rpm. Supernatants were diluted to a volume of 1 mL with 30:70 v/v acetonitrile:water. All diluted supernatants were analyzed by UPLC using a Waters Acquity UPLC with a PDA UV detector. The results are shown in Table II.

TABLE I1

| Compound No | pH 7 Solubility (mg/mL) |
|---|---|
| 1 | 0.1 |
| 2 | 0.14 |
| 8 | >13 |
| 9 | ND |
| 10 | ND |
| 13 | 4.48 |
| 19 | 0.11 |
| 20 | 0.01 |
| 21 | 0.37 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A compound of Formula I: 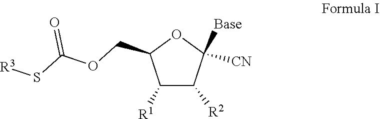
or a pharmaceutically acceptable salt thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O— or —OCR⁶O—;
R³, R⁴, and R⁵ are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R³, R⁴, and R⁵ are each, independently, optionally substituted with one, two or three substituents independently selected from the group consisting of halogen, cyano, —N₃, —OR⁷, —NR⁸R⁹, and phenyl optionally substituted with one, two or three substituents independently selected from halo, cyano, and C₁-C₆ alkyl;
R⁶ is H, C₁-C₆ alkyl or C₆-C₁₀ aryl;
each R⁷ is independently H, C₁-C₆ alkyl, C₁-C₆ haloalkyl, and C₃-C₆ cycloalkyl;
each R⁸ is independently H, C₁-C₆ alkyl, C₁-C₆ haloalkyl, and C₃-C₆ cycloalkyl;
each R⁹ is independently H, C₁-C₆ alkyl, C₁-C₆ haloalkyl, and C₃-C₆ cycloalkyl;
Base is 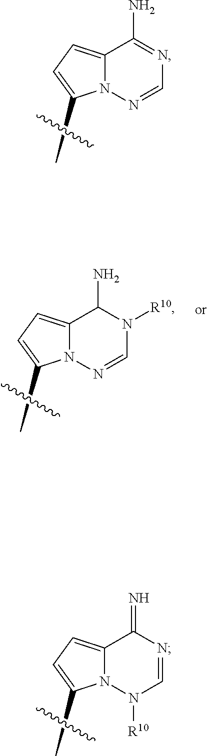
and
R¹⁰ is C₁-C₆ alkyl substituted with —OP(O)(OH)₂.

2. The compound of claim 1, or the pharmaceutically acceptable salt thereof, wherein the compound is of Formula Ia:

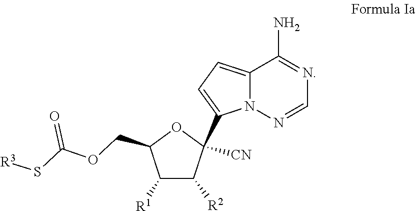

3. (canceled)

4. (canceled)

5. The compound of claim 1, or the pharmaceutically acceptable salt thereof, wherein R¹ is —OH and R² is —OH.

6. (canceled)

7. (canceled)

8. The compound of claim 1, or the pharmaceutically acceptable salt thereof, wherein R¹ is —OC(═O)R⁴ and R² is —OC(═O)R⁵.

9. (canceled)

10. (canceled)

11. The compound of claim 1, or the pharmaceutically acceptable salt thereof, wherein R⁴ and R⁵ are each independently C₁-C₈ alkyl.

12. (canceled)

13. (canceled)

14. The compound of claim 1, or the pharmaceutically acceptable salt thereof, wherein R³ is C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S.

15.-24. (canceled)

25. The compound of claim 1, or the pharmaceutically acceptable salt thereof, wherein the compound is selected from:

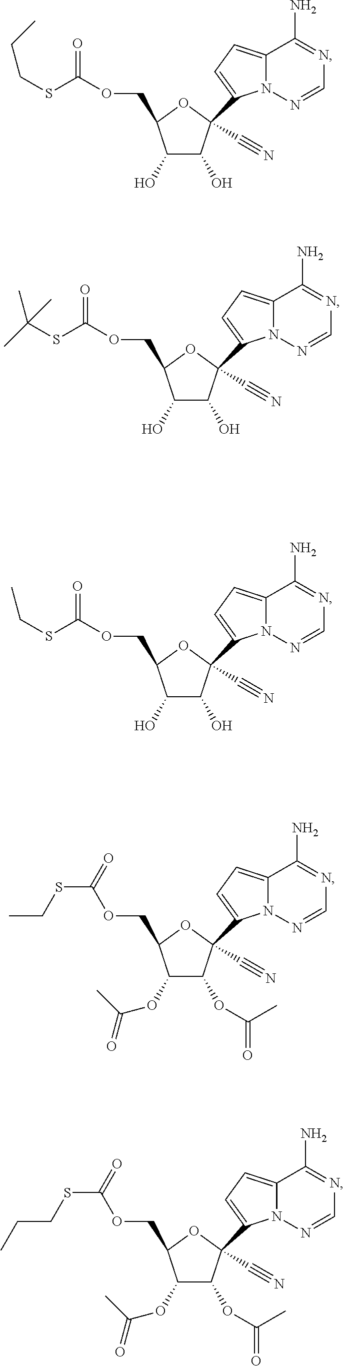 [SMILES: CCCSC(=O)OC[C@H]1O[C@@](C#N)(c2ccc3c(N)ncnn23)[C@H](O)[C@@H]1O ; CC(C)(C)SC(=O)OC[C@H]1O[C@@](C#N)(c2ccc3c(N)ncnn23)[C@H](O)[C@@H]1O ; CCSC(=O)OC[C@H]1O[C@@](C#N)(c2ccc3c(N)ncnn23)[C@H](O)[C@@H]1O ; CCSC(=O)OC[C@H]1O[C@@](C#N)(c2ccc3c(N)ncnn23)[C@H](OC(C)=O)[C@@H]1OC(C)=O ; CCCSC(=O)OC[C@H]1O[C@@](C#N)(c2ccc3c(N)ncnn23)[C@H](OC(C)=O)[C@@H]1OC(C)=O]

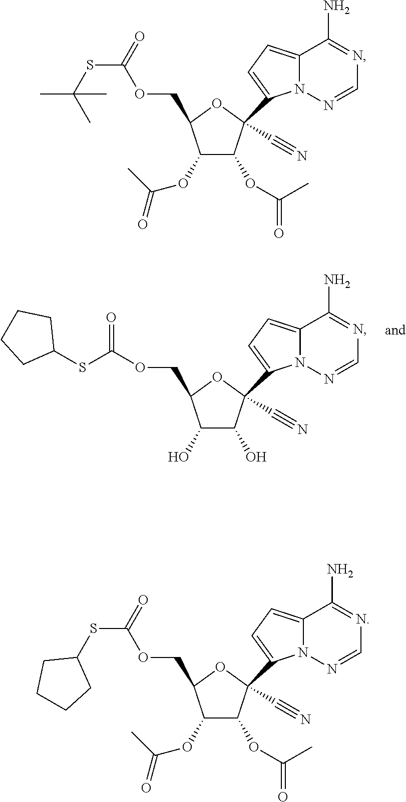 [SMILES: CC(=O)O[C@H]1[C@@H](OC(C)=O)[C@](C#N)(c2ccc3c(N)ncnn23)O[C@@H]1COC(=O)SC(C)(C)C ; N#C[C@@]1(c2ccc3c(N)ncnn23)O[C@H](COC(=O)SC2CCCC2)[C@@H](O)[C@H]1O ; CC(=O)O[C@H]1[C@@H](OC(C)=O)[C@](C#N)(c2ccc3c(N)ncnn23)O[C@@H]1COC(=O)SC1CCCC1]

26. A compound of Formula A: 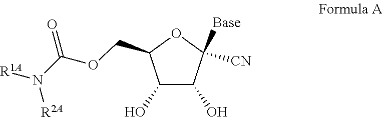
or a pharmaceutically acceptable salt thereof, wherein:
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —C(═O)R^{3A}, —C(═O)OR^{3A}, —NR^{4A}R^{5A}, C₁-C₆ alkyl, and phenyl;
wherein the substituent C₁-C₆ alkyl of R^{1A} and R^{2A} is optionally substituted with one, two or three substituents independently selected from halo, cyano, and phenyl; and
the substituent phenyl of R^{1A} and R^{2A} is optionally substituted with one, two or three substituents independently selected from halo, cyano, and C₁-C₆ alkyl; or
R^{1A} is H;
R^{2A} is C₁-C₈ alkyl substituted with —C(═O)OR^{3A};
wherein C₁-C₈ alkyl substituted with —C(═O)OR^{3A} of R^{2A} is optionally further substituted with one or two substituents independently selected from halo, cyano, and phenyl, and —OR^{3A};
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S;
each R^{4A} is independently H, C₁-C₆ alkyl, C₁-C₆ haloalkyl, and C₃-C₆ cycloalkyl;
each R^{5A} is independently H, C₁-C₆ alkyl, C₁-C₆ haloalkyl, and C₃-C₆ cycloalkyl;
Base is 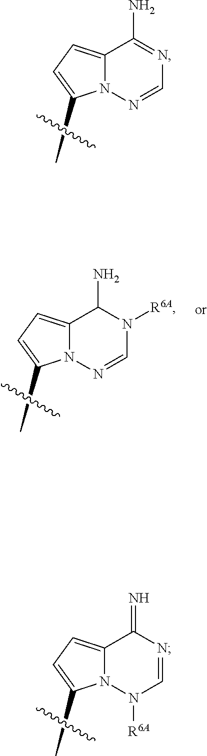
and
R^{6A} is C₁-C₆ alkyl substituted with —OP(O)(OH)₂.

27. The compound of claim 26, or the pharmaceutically acceptable salt thereof, wherein the compound is of Formula Aa:

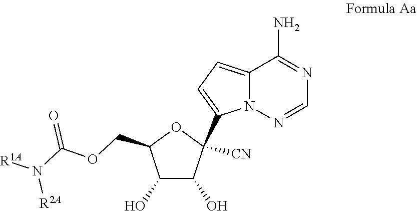

28. The compound of claim 26, or the pharmaceutically acceptable salt thereof, wherein
R^{1A} is H;
R^{2A} is C₁-C₈ alkyl substituted with COOR^{3A};
wherein C₁-C₈ alkyl substituted with —C(═O)OR^{3A} of R^{2A} is optionally further substituted with one or two substituents independently selected from halo, cyano, phenyl, and —OR^{3A}; and
each R^{3A} is independently H, C₁-C₆ alkyl, C₁-C₆ haloalkyl, C₃-C₆ cycloalkyl, or 3 to 8 membered heterocyclyl containing 1, 2, or 3 heteroatoms selected independently from N, O, and S.

29.-31. (canceled)

32. The compound of claim 26, or the pharmaceutically acceptable salt thereof, wherein the compound is

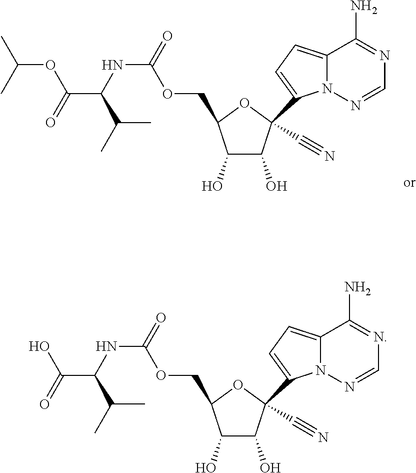 [SMILES: CC(C)OC(=O)[C@@H](NC(=O)OC[C@H]1O[C@@](C#N)(c2ccc3c(N)ncnn23)[C@H](O)[C@@H]1O)C(C)C ; CC(C)[C@H](NC(=O)OC[C@H]1O[C@@](C#N)(c2ccc3c(N)ncnn23)[C@H](O)[C@@H]1O)C(=O)O]

33. The compound of claim 26, wherein
R^{1A} and R^{2A} are joined to form a 3 to 8 membered heterocyclyl containing 1, 2, or 3 heteroatoms selected independently from N, O, and S or 5 to 10 membered heteroaryl containing 1, 2, or 3 heteroatoms selected form N, O, and S;
wherein the 3 to 8 membered heterocyclyl or 5 to 10 membered heteroaryl of R^{1A} and R^{2A} is optionally substituted with one, two or three substituents independently selected from the group consisting of halogen, cyano, oxo, —N₃, —OR^{3A}, —COR^{3A}, —COOR^{3A}, —NR^{4A}R^{5A}, C₁-C₆ alkyl, and phenyl;
wherein the substituent C₁-C₆ alkyl of R^{1A} and R^{2A} is optionally substituted with one, two or three substituents independently selected from halo, cyano, and phenyl; and
the substituent phenyl of R^{1A} and R^{2A} is optionally substituted with one, two or three substituents independently selected from halo, cyano, and C₁-C₆ alkyl;
each R^{3A} is independently H, C₁-C₆ alkyl, C₁-C₆ haloalkyl, C₃-C₆ cycloalkyl, and 3 to 8 membered heterocyclyl containing 1, 2, or 3 heteroatoms selected independently from N, O, and S;
each R^{4A} is independently H, C₁-C₆ alkyl, C₁-C₆ haloalkyl, and C₃-C₆ cycloalkyl; and
each R^{5A} is independently H, C₁-C₆ alkyl, C₁-C₆ haloalkyl, and C₃-C₆ cycloalkyl.

34. (canceled)

35. The compound of claim 26, wherein the compound is selected from:

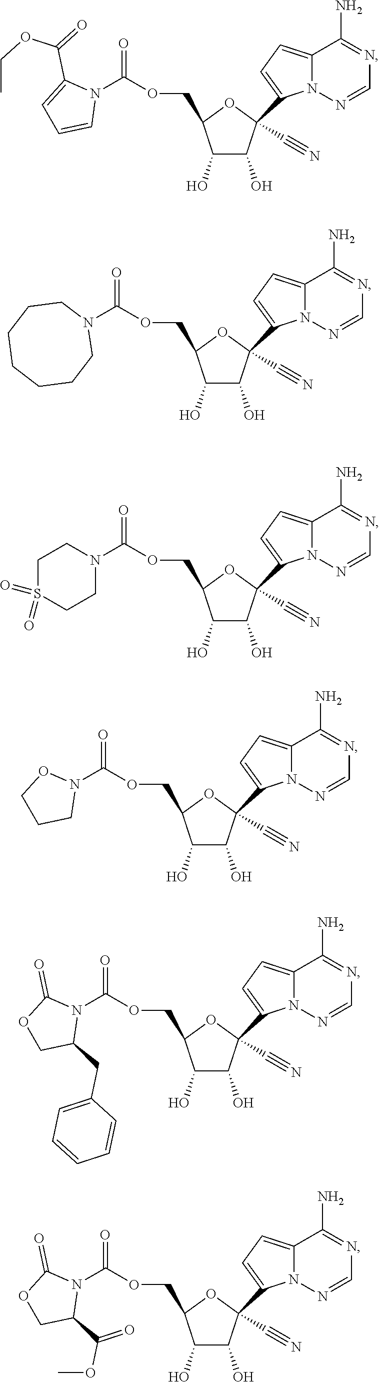 [SMILES: CCOC(=O)c1cccn1C(=O)OC[C@H]1O[C@@](C#N)(c2ccc3c(N)ncnn23)[C@H](O)[C@@H]1O ; N#C[C@@]1(c2ccc3c(N)ncnn23)O[C@H](COC(=O)N2CCCCCCC2)[C@@H](O)[C@H]1O ; N#C[C@@]1(c2ccc3c(N)ncnn23)O[C@H](COC(=O)N2CCS(=O)(=O)CC2)[C@@H](O)[C@H]1O ; N#C[C@@]1(c2ccc3c(N)ncnn23)O[C@H](COC(=O)N2CCCO2)[C@@H](O)[C@H]1O ; N#C[C@@]1(c2ccc3c(N)ncnn23)O[C@H](COC(=O)N2C(=O)OC[C@@H]2Cc2ccccc2)[C@@H](O)[C@H]1O ; COC(=O)[C@H]1COC(=O)N1C(=O)OC[C@H]1O[C@@](C#N)(c2ccc3c(N)ncnn23)[C@H](O)[C@@H]1O]

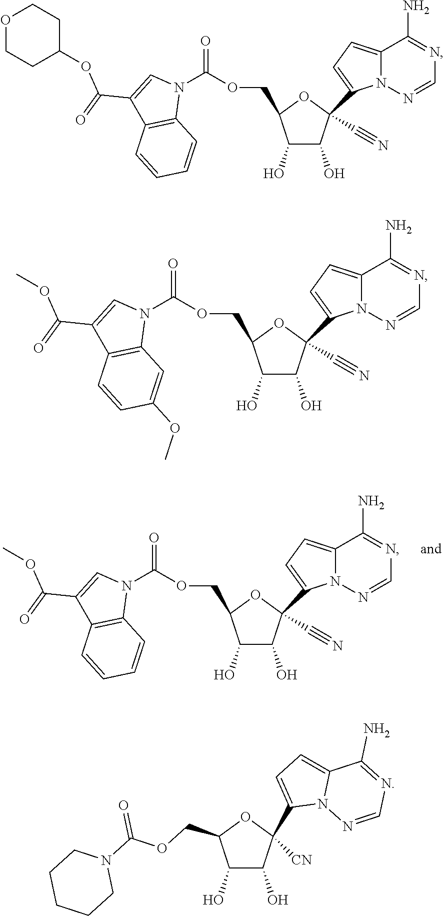 [SMILES: N#C[C@@]1(c2ccc3c(N)ncnn23)O[C@H](COC(=O)n2cc(C(=O)OC3CCOCC3)c3ccccc32)[C@@H](O)[C@H]1O ; COC(=O)c1cn(C(=O)OC[C@H]2O[C@@](C#N)(c3ccc4c(N)ncnn34)[C@H](O)[C@@H]2O)c2cc(OC)ccc12 ; COC(=O)c1cn(C(=O)OC[C@H]2O[C@@](C#N)(c3ccc4c(N)ncnn34)[C@H](O)[C@@H]2O)c2ccccc12 ; N#C[C@@]1(c2ccc3c(N)ncnn23)O[C@H](COC(=O)N2CCCCC2)[C@@H](O)[C@H]1O]

36. A compound of Formula B: 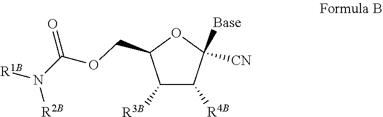
or a pharmaceutically acceptable salt thereof, wherein:
R^{3B} is —OC(═O)R^{5B} or —OC(═O)OR^{5B};
R^{4B} is —OC(═O)R^{6B} or —OC(═O)OR^{6B}; or
R^{3B} and R^{4B} are taken together to form —OC(═O)O— or —OCHR^{7B}O;
R^{5B} and R^{6B} are each independently C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{5B} and R^{6B} are each, independently, optionally substituted with one, two or three substituents independently selected from the group consisting of halogen, cyano, —N₃, —OR^{8B}, —NR^{9B}R^{10B}, and phenyl optionally substituted with one, two or three substituents independently selected from halo, cyano, and C₁-C₆ alkyl;
each R^{8B} is independently H, C₁-C₆ alkyl, C₁-C₆ haloalkyl, and C₃-C₆ cycloalkyl;
each R^{9B} is independently H, C₁-C₆ alkyl, C₁-C₆ haloalkyl, and C₃-C₆ cycloalkyl;
each R^{10B} is independently H, C₁-C₆ alkyl, C₁-C₆ haloalkyl, and C₃-C₆ cycloalkyl;
R^{7B} is H, C₁-C₆ alkyl or C₆-C₁₀ aryl;
R^{1B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{1B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl;
R^{2B} is H, C₁-C₈ alkyl, C₂-C₈ alkenyl, C₂-C₈ alkynyl, C₃-C₈ carbocyclyl, C₆-C₁₀ aryl, or 5 to 6 membered heteroaryl containing 1, 2, or 3 heteroatoms selected form N, O, and S;
wherein C₁-C₈ alkyl, C₂-C₈ alkenyl, C₂-C₈ alkynyl, C₃-C₈ carbocyclyl, C₆-C₁₀ aryl, or 5 to 6 membered heteroaryl of R^{2B} is optionally substituted with one, two or three substituents independently selected from the group consisting of halogen, cyano, —N₃, —OR^{11B}, —C(═O)R^{11B}, —C(═O)OR^{11B}, —NR^{12B}R^{13B}, and phenyl optionally substituted with one, two or three substituents independently selected from halo, cyano, and C₁-C₆ alkyl; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of halogen, cyano, oxo, —N³, —OR^{11B}, —C(═O)R^{11B}, —C(═O)OR^{11B}, —NR^{12B}R^{13B}, C₁-C₆ alkyl, and phenyl;
wherein the substituent C₁-C₆ alkyl of R^{1B} and R^{2B} is optionally substituted with one, two or three substituents independently selected from halo, cyano, and phenyl; and
the substituent phenyl of R^{1B} and R^{2B} is optionally substituted with one, two or three substituents independently selected from halo, cyano, and C₁-C₆ alkyl;
each R^{11B} is independently H, C₁-C₆ alkyl, C₁-C₆ haloalkyl, C₃-C₆ cycloalkyl and 3 to 8 membered heterocyclyl containing 1, 2, or 3 heteroatoms selected independently from N, O, and S;
each R^{12B} is independently H, C₁-C₆ alkyl, C₁-C₆ haloalkyl, and C₃-C₆ cycloalkyl;
each R^{13B} is independently H, C₁-C₆ alkyl, C₁-C₆ haloalkyl, and C₃-C₆ cycloalkyl;
Base is 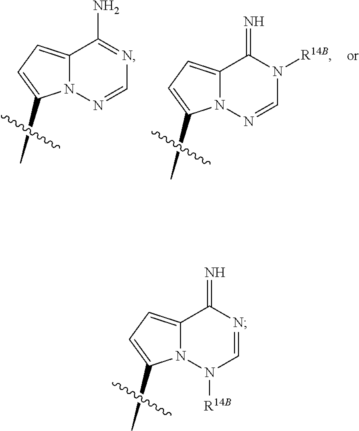
and
R^{14B} is C₁-C₆ alkyl substituted with —OP(O)(OH)₂.

37.-39. (canceled)

40. The compound of claim 36, or the pharmaceutically acceptable salt thereof, wherein the compound is of Formula Ba:

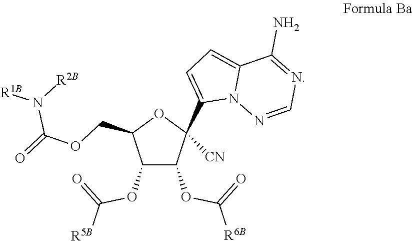

41.-44. (canceled)

45. The compound of claim 36, or the pharmaceutically acceptable salt thereof, wherein R^{5B} and R^{6B} are each independently C₁-C₃ alkyl.

46.-49. (canceled)

50. The compound of claim 36, or the pharmaceutically acceptable salt thereof, wherein
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, C₁-C₆ alkyl, and phenyl;
wherein the substituent C₁-C₆ alkyl of R^{1B} and R^{2B} is optionally substituted with one, two or three substituents independently selected from halo, cyano; and phenyl; and
each R^{11B} is independently H, C₁-C₆ alkyl, C₃-C₆ cycloalkyl and 3 to 8 membered heterocyclyl containing 1, 2, or 3 heteroatoms selected independently from N, O, and S.

51.-53. (canceled)

54. The compound of claim 36, or the pharmaceutically acceptable salt thereof, wherein
R^{1B} is H;
R^{2B} is C₁-C₆ alkyl optionally substituted with one, two or three substituents independently selected from the group consisting of halogen, —OR^{11B}, and —C(═O)OR^{11B}; or
R^{1B} and R^{2B} are joined to form a 3 to 8 membered heterocyclyl containing 1, 2, or 3 heteroatoms selected independently from N, O, and S, or 5 to 10 membered heteroaryl containing 1, 2, or 3 heteroatoms selected form N, O, and S;
wherein the 3 to 8 membered heterocyclyl or 5 to 10 membered heteroaryl of R^{1B} and R^{2B} is optionally substituted with one, two or three substituents independently selected from the group consisting of oxo, —OR^{11B}, —C(═O)OR^{11B}, and C₁-C₃ alkyl;
wherein the substituent C₁-C₃ alkyl of R^{1B} and R^{2B} is optionally substituted with one, two or three substituents independently selected from halo, cyano; and phenyl; and
each R^{11B} is independently H, C₁-C₃ alkyl, C₃-C₆ cycloalkyl and 3 to 8 membered heterocyclyl containing 1, 2, or 3 heteroatoms selected independently from N, O, and S.

55. The compound of claim 36, or the pharmaceutically acceptable salt thereof, wherein the compound is selected from:

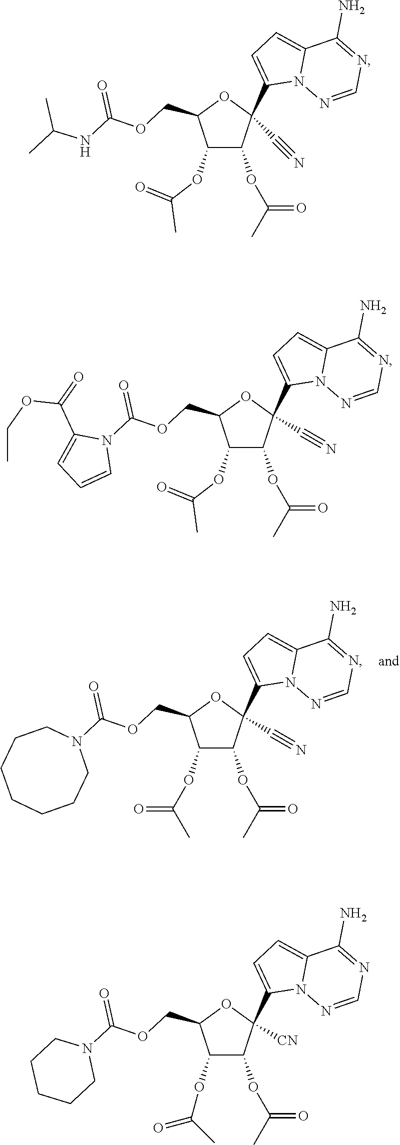 [SMILES: CC(=O)O[C@H]1[C@@H](OC(C)=O)[C@](C#N)(c2ccc3c(N)ncnn23)O[C@@H]1COC(=O)NC(C)C ; CCOC(=O)c1cccn1C(=O)OC[C@H]1O[C@@](C#N)(c2ccc3c(N)ncnn23)[C@H](OC(C)=O)[C@@H]1OC(C)=O ; CC(=O)O[C@H]1[C@@H](OC(C)=O)[C@](C#N)(c2ccc3c(N)ncnn23)O[C@@H]1COC(=O)N1CCCCCCC1 ; CC(=O)O[C@H]1[C@@H](OC(C)=O)[C@](C#N)(c2ccc3c(N)ncnn23)O[C@@H]1COC(=O)N1CCCCC1]

56. The compound of claim 36, or the pharmaceutically acceptable salt thereof, wherein the compound is selected from:

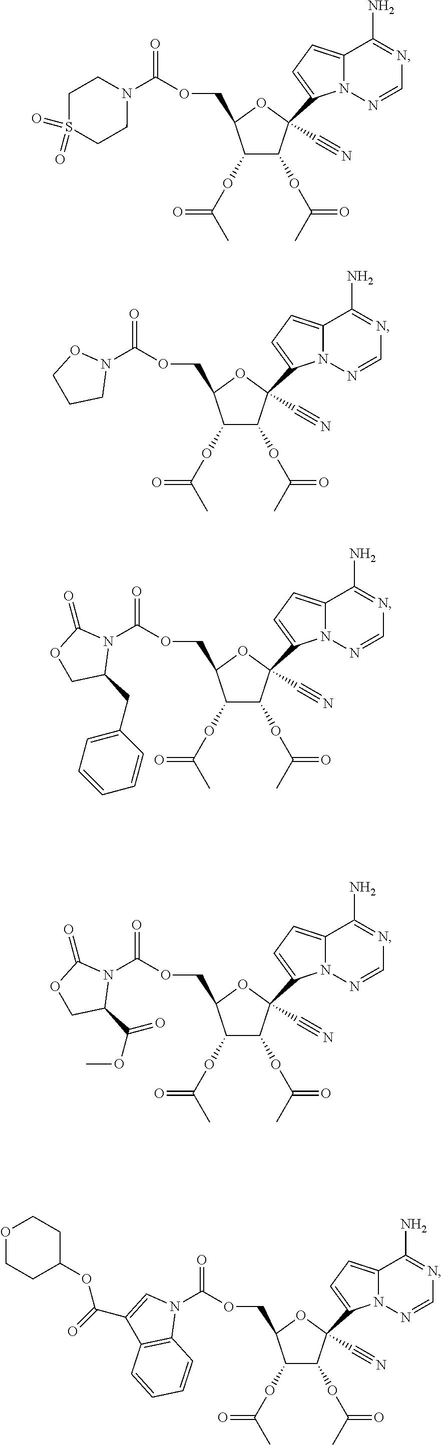 [SMILES: CC(=O)O[C@H]1[C@@H](OC(C)=O)[C@](C#N)(c2ccc3c(N)ncnn23)O[C@@H]1COC(=O)N1CCS(=O)(=O)CC1 ; CC(=O)O[C@H]1[C@@H](OC(C)=O)[C@](C#N)(c2ccc3c(N)ncnn23)O[C@@H]1COC(=O)N1CCCO1 ; CC(=O)O[C@H]1[C@@H](OC(C)=O)[C@](C#N)(c2ccc3c(N)ncnn23)O[C@@H]1COC(=O)N1C(=O)OC[C@@H]1Cc1ccccc1 ; COC(=O)[C@H]1COC(=O)N1C(=O)OC[C@H]1O[C@@](C#N)(c2ccc3c(N)ncnn23)[C@H](OC(C)=O)[C@@H]1OC(C)=O ; CC(=O)O[C@H]1[C@@H](OC(C)=O)[C@](C#N)(c2ccc3c(N)ncnn23)O[C@@H]1COC(=O)n1cc(C(=O)OC2CCOCC2)c2ccccc21]

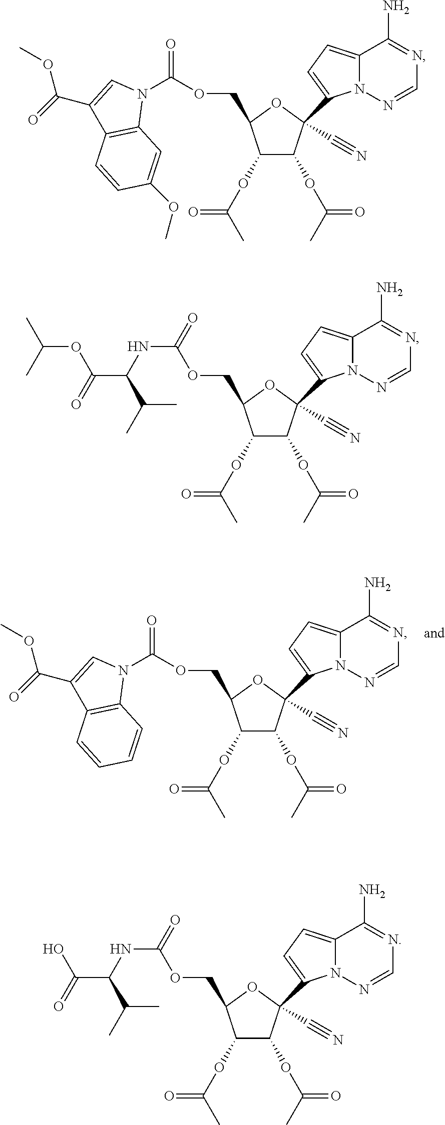 [SMILES: COC(=O)c1cn(C(=O)OC[C@H]2O[C@@](C#N)(c3ccc4c(N)ncnn34)[C@H](OC(C)=O)[C@@H]2OC(C)=O)c2cc(OC)ccc12 ; CC(=O)O[C@H]1[C@@H](OC(C)=O)[C@](C#N)(c2ccc3c(N)ncnn23)O[C@@H]1COC(=O)N[C@H](C(=O)OC(C)C)C(C)C ; COC(=O)c1cn(C(=O)OC[C@H]2O[C@@](C#N)(c3ccc4c(N)ncnn34)[C@H](OC(C)=O)[C@@H]2OC(C)=O)c2ccccc12 ; CC(=O)O[C@H]1[C@@H](OC(C)=O)[C@](C#N)(c2ccc3c(N)ncnn23)O[C@@H]1COC(=O)N[C@H](C(=O)O)C(C)C]

57. A pharmaceutical composition comprising the compound of claim 1, or a pharmaceutically acceptable salt thereof, and one or more pharmaceutically acceptable excipients.

58. (canceled)

59. (canceled)

60. A method of treating or preventing a viral infection in a human in need thereof, wherein the method comprises administering to the human the compound of claim 1, or a pharmaceutically acceptable salt thereof.

61.-97. (canceled)

98. A method of treating or preventing a viral infection in a human in need thereof, wherein the method comprises administering to the human the compound of claim 26, or a pharmaceutically acceptable salt thereof.

99. A method of treating or preventing a viral infection in a human in need thereof, wherein the method comprises administering to the human the compound of claim 36, or a pharmaceutically acceptable salt thereof.
